(19) United States
(12) Patent Application Publication
(10) Pub. No.: US 2025/0027932 A1
(43) Pub. Date: Jan. 23, 2025

(54) DIFFERENTIATION OF HUMAN TISSUES WITHOUT CILIA FROM PLURIPOTENT STEM CELLS

(71) Applicant: University of Washington, Seattle, WA (US)

(72) Inventor: Benjamin Freedman, Seattle, WA (US)

(73) Assignee: University of Washington, Seattle, WA (US)

(21) Appl. No.: 18/713,882

(22) PCT Filed: Nov. 30, 2022
(86) PCT No.: PCT/US2022/080710

§ 371 Date: May 28, 2024

Related U.S. Application Data
(60) Provisional application No. 63/284,556, filed on Nov. 30, 2021.

(51) Int. Cl. G01N 33/50; C12N 5/071; C12N 9/22; C12N 15/11; C12N 15/90

(52) U.S. Cl. CPC G01N 33/5082; C12N 5/0684; C12N 9/22; C12N 15/11; C12N 15/907; C12N 2310/20; C12N 2506/45

(57) ABSTRACT

Embodiments of the present disclosure provide compositions and methods for making a genetically modified pluripotent stem cell, wherein the genetically modified pluripotent stem cell lacks cilia. Embodiments of the present disclosure also provide compositions and methods for using the genetically modified pluripotent stem cell to generate genetically modified organoids, wherein the genetically modified organoids lack cilia.

| | | Knockout | Isogenic |
|---|---|---|---|
| Gene | guide RNA | Mutants | Controls |
| KIF3A | CATATGGACAAACCGGAAC | 7 | 7 |
| KIF3B | TTCGCTGTCGGCCCATGAA | 3 | 3 |
| | TACACCATGGAAGGAATCCG | 4 | 2 |

### CROSS-REFERENCE TO RELATED APPLICATION

This application claims the benefit of U.S. Provisional Patent Application No. 63/284,556 filed Nov. 30, 2021. The content of this above-identified U.S. provisional application is incorporated herein in their entirety by this reference.

### STATEMENT OF GOVERNMENT LICENSE RIGHTS

This invention was made with government support under Grant Nos. RO1DK117914, UG3TR000504, UG3TR002158, and U01DK127553 awarded by the National Institutes of Health (NIH). The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3915-P1279WOUW_Seq_List_20221130. The XML file is 32 KB; was created on Nov. 30, 2022; and is being submitted via Patent Center with the filing of the specification.

### BACKGROUND

Cilia are antenna-like organelles found in eukaryotic cells. There are two major types of cilia, namely motile and non-motile cilia. In animals, non-motile primary cilia are found on nearly every type of cell. Cilia have physiological roles in chemo-sensation, signal transduction, and cell growth control, thus having importance in cell function. Furthermore, cilia play roles in the function of many human organs. Some of the signaling within cilia occurs through ligand binding such as in a Hedgehog signaling pathway. This pathway transmits information to embryonic cells required for proper cell differentiation. Different parts of the embryo have different concentrations of hedgehog signaling proteins. The pathway also has roles in adults and cancer. Mutations in genes that affect cilia structure and function cause a spectrum of inherited disorders called the ciliopathies which include, for example, polycystic kidney disease and other organ-specific phenotypes.

Understanding and analyzing Hedgehog signaling and other protein pathways in relation to development and disease is important to create drugs and therapies that will ultimately help treat and cure these dysfunctions and diseases. The present disclosure addresses these and other needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the foregoing, in one aspect of the invention, the disclosure provides for isolated genetically modified pluripotent stem cells (PSCs) comprising a disruption in a gene encoding at least one subunit of kinesin-2, wherein the genetic modification results in PSC lacking a ciliary structure. In some embodiments, the genetic modification comprises disruption of KIF3A and reduced expression of KIF3A protein, wherein reduced expression of KIF3A protein results in PSCs that lack ciliary structures.

In accordance with the foregoing, in one aspect the disclosure provides a genetically modified cell descended from the genetically modified pluripotent stem cell (PSC), as described above.

In accordance with the foregoing, in one aspect the disclosure provides an isolated genetically modified organoid derived from the genetically modified pluripotent stem cells (PSCs), as described above.

In accordance with the foregoing, in one aspect the disclosure provides a method for making an isolated pluripotent stem cell (PSC) lacking a ciliary structure. In some embodiments, the method can comprise (a) providing the PSC comprising a target gene encoding at least one subunit of kinesin-2; and (b) genetically modifying the PSC to contain a disruption of the target gene encoding at least one subunit of kinesin-2, wherein the disruption produces a genetically modified PSC lacking the ciliary structure.

In accordance with the foregoing, in one aspect the disclosure provides a method of differentiating an isolated genetically modified tubular organoid derived from a genetically modified PSC. In some embodiments the method can comprise (a) providing a quantity of genetically modified pluripotent stem cells (PSCs) as described above; (b) culturing the genetically modified PSC in a first culture medium comprising a ROC kinase inhibitor for at least 24 hours and then culturing the PSC sandwiched between two layers of a gelatinous protein mixture secreted by Engelbreth-Holm-Swarm mouse sarcoma cells to form epiblast spheroids, wherein the first medium does not comprise exogenous fibroblast growth factor 2 (FGF2), activin or bone morphogenetic protein; and (c) contacting the epiblast spheroids from step (b) with a second culture medium comprising at least 12 μM CHIR99021 for at least 24 hours, wherein the second medium does not comprise exogenous fibroblast growth factor 2 (FGF2), activin or bone morphogenetic protein, and (d) culturing the epiblast spheroids from step (c) with a third culture medium comprising B27 for at least 48 hours, wherein the third medium does not comprise exogenous fibroblast growth factor 2 (FGF2), activin or bone morphogenetic, thereby differentiating the epiblast spheroids into genetically modified tubular organoids.

In accordance with the foregoing, in one aspect the disclosure provides a method for testing the effects of compound candidates on a phenotypic organoid model. In some embodiments, the method can comprise generating the phenotypic organoid model on a screening platform comprising the steps of: (a) plating each of a plurality of wells of a culture vessel with a population of genetically modified pluripotent stem cells (PSCs) as described above; (b) differentiating the population of genetically modified PSCs plated in each of the plurality of wells using a single induction step without dissociating or replating the differentiated cells; treating the population of genetically modified PSCs plated in each of the plurality of wells with a therapeutic compound candidate; and testing one or more effects resulting from treatment with each of the therapeutic compound candidates; wherein the method is performed manually using pipettes or automatically by a liquid handling robot.

In accordance with the foregoing, in one aspect the disclosure provides a method for measuring organ specific toxicity and disease phenotypes of an agent on a genetically modified organoid, the method comprising: (a) providing one or more genetically modified organoids derived from a genetically modified pluripotent stem cell (PSC) in a high throughput format; (b) admixing the agent with the one or more genetically modified organoids; and (c) detecting one or more outcomes of agent on the one or more genetically modified organoids wherein the one or more outcomes indicates toxicity, disease, differentiation state, or a combination thereof of the one or more genetically modified organoids.

In accordance with the foregoing, in one aspect the disclosure provides a method of screening a compound for an effect on tubular organoids, including providing a quantity of tubular organoids, adding one or more compounds to the tubular organoids, determining changes to phenotype or activity of the tubular organoids, and correlating the changes with an effect of the compounds on tubular organoids, thereby screening the one or more compounds for an effect on tubular organoids.

In accordance with the foregoing, in one aspect the disclosure provides a method of identifying a disrupted signaling pathway contributing to polycystic kidney disease (PKD), the method comprising: (a) differentiating genetically modified kidney organoids to day 18; (b) performing transcriptome analysis and/or proteomic analysis on the partitioned 18 day old genetically modified kidney organoids; (c) using transcriptome analysis and/or proteomic analysis to identify at least one differentially expressed gene; (d) comparing results from the transcriptome analysis and/or the proteomic analysis from step (c) to transcriptome analysis and/or proteomic analysis results of: (a) a non-genetically modified kidney organoid; (b) a PKD knockout; and (c) the non-genetically modified kidney organoid to a PKD knockout; identifying a signaling pathway that is expressed in (i) the genetically modified kidney organoid and (ii) determining whether the signaling pathway is similar to a disrupted pathway in PKD, thereby identifying a signaling pathway contributing to PKD.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1E. A CRISPR gene editing strategy produces stable hPSCs completely lacking cilia. (a) Summary of new hPSC lines. KIF3A (SEQ ID NO: 1); KIF3B (top, SEQ ID NO: 2) (bottom, SEQ ID NO: 3). (b) Representative KIF3A and KIF3B chromatograms. Two genetic backgrounds (H9 and WTC11) were used to generate isogenic pairs. The sgRNA sequence used for CRISPR targeting is highlighted. Control, top left box (SEQ ID NO: 11); Control top right box (SEQ ID NO: 12); H9 KIF3A Mutant 2 (SEQ ID NO: 13); and WTC11 KIF3B Mutant 2 (SEQ ID NO: 14). (c) KIF3A and KIF3B immunoblots showing multiple distinct control, KIF3A^{-/-}, and KIF3B^{-/-} hPSC lines. (d) Confocal immunofluorescence images of ARL13B and acetylated a-tubulin (AcTub) with ZO-1 counterstain in representative fields of undifferentiated control and kinesin-2 knockout hPSCs, on two distinct genetic backgrounds (H9 ES cells or WTC11 iPS cells). Representative ciliary axonemes are indicated with arrowheads. (e) Cilia counts in undifferentiated control and kinesin-2 hPSCs on two distinct genetic backgrounds (mean±s.e.m., n≥3 independent biological replicates per condition from a total of 24 distinct cell lines; ≥180 cells counted per cell line). Scale bars, 20 μm. ****, p<0.0001; ***, p<0.001.

FIGS. 2A-2I. KIF3A^{-/-} and KIF3B^{-/-} hPSCs establish a renewable source of diverse human cell types lacking cilia. (a) hPSC colony morphology at passages >30. No abnormalities were observed in earlier or later passages. (b) Cell viability assay (alamarBlue). Identical numbers of KIF3A^{-/-} or KIF3B^{-/-} hPSCs or isogenic controls were plated on day 0 and relative fluorescence units (RFU) were quantified at the indicated times points (mean±s.e.m., n=3 independent biological replicates per condition from a total of 9 distinct cell lines). (c) Average cell number at passage, after plating identical numbers of cells (mean±s.e.m., n≥7 biological replicates per condition from a total of 11 distinct cell lines). (d) Summary of clonal abnormalities and representative karyotypes from KIF3A^{-/-} or KIF3B^{-/-} hPSCs, compared to isogenic control (mean of n=10 cells from two distinct cell lines per condition). (e) Confocal sections showing immunofluorescence for pluripotency, polarity, and ciliary markers in hPSC epiblast spheroids. Scale bars, 20 μm. (f) Representative images and (g) quantification of EB formation in control and KIF3A^{-/-} or KIF3B^{-/-} knockout hPSCs (n≥3 biological replicates per condition from a total of 8 distinct cell lines). Scale bars, 500 μm. (h) Confocal images and (i) quantification of cilia formation in EB epithelial cells expressing the tight junction marker zonula occludens 1 (ZO-1). Scale bars, 20 μm (n≥2 independent biological replicates from a total of 9 distinct cell lines. ***, p<0.001).

FIGS. 3A-3H. Cilia are critical for terminal differentiation in vivo. (a) Weights of growths removed from animals (mean±s.e.m., n≥6 independent biological replicates per condition from a total of 14 distinct cell lines). (b) Representative images and (c-d) quantification of tissue types in growth sections stained with hematoxylin and eosin. Labels indicate pigmented epithelium (pi), secretory epithelium (se), and cartilage (ca) regions. (mean±s.e.m., n≥5 independent biological replicates per condition from a total of 14 distinct cell lines; **, p<0.01.) (e) Representative wide-field and (f) confocal microscope images showing ARL13B, with (g) averaged line scans drawn from neuroepithelium into rosette lumens (mean±s.e.m., n≥9 independent biological replicates per condition from a total of 8 distinct cell lines). White dashed arrow illustrates how line scans were drawn. (h) Representative confocal images showing neuronal marker immunofluorescence in teratomas. Scale bar, 100 μm (b,e) or 10 μm (f,h).

FIGS. 4A-4F. KIF3A^{-/-} and KIF3B^{-/-} kidney organoids express defects in neuronal and nephron differentiation. (a) Representative confocal immunofluorescence for markers of neurons and proximal tubules in control, KIF3A^{-/-}, and KIF3B^{-/-} kidney organoid cultures, showing whole 96-wells or (b) higher-magnification fields. A small minority of SOX2⁺ cells are found in proximal tubules (arrowhead). In addition to tubules, fainter LTL staining is observed in neuroepithelial structures (arrows). (c) Quantification of TUJ1⁺ neuron area in these 96-wells (mean±s.e.m., n≥3 independent biological replicates per condition from a total of 11 distinct cell lines. **, p<0.01). (d) Confocal immunofluorescence images of nephron markers in kidney organoid differentiations in representative 96-well plates and (e) higher magnification. Each kidney organoid contains distal tubular (ECAD⁺), proximal tubular (LTL⁺), and podocyte (PODXL⁺) epithelial cells. (f) Quantification of kidney organoid differentiation in these lines (mean±s.e.m., n≥7 independent biological replicates per condition from a total of 13 distinct cell lines. **, p<0.01; ***, p<0.001).

FIGS. 5A-5F. Modulation of hedgehog pathway activation via kinesin-2 promotes kidney organoid differentiation. (a) Heatmap of the expression level of genes differentially expressed as a function KIF3B^{-/-} loss in hPSCs (FDR<0.1). Boxes to the right: Enriched gene sets from gene set analysis using the MSigDB Hallmarks gene set collection (hypergeometric test, FDR<0.1). (b) Schematic of organoid differentiation time course, with sampling intervals indicated (red arrows). (c) Representative GLI3 and (d) GLI1 immunoblots of control versus KIF3A^{-/-} (top) and KIF3B^{-/-} (bottom) organoid lysates on days 0-7 of differentiation. (e) Representative GLI3 and (f) PTCH1 immunoblots of undifferentiated hPSCs (day 0) and terminally differentiated organoids (day 18) in KIF3A^{-/-} and KIF3B^{-/-} genotypes (3A, and 3B; subscripts indicate gRNAs 1 or 2, respectively), compared to paired isogenic controls (#1-#3).

FIGS. 6A-6H. Smoothened agonist perturbs organoid differentiation. (a) Schematic of organoid differentiation time course, indicating initiation of SAG treatment and sampling time points. (b) Representative GLI3 and (c) GLI1 immunoblots of cultures on day 7 differentiation, under standard conditions (−) or supplemented with 1 μM SAG (S) starting on day 1.5 of differentiation. (d) Representative GLI3 immunoblot of control, KIF3A^{-/-}, and KIF3B^{-/-} organoid lysates on day 18 of differentiation. For each genotype, conditions were untreated (−), +1 μM SAG (S), or +10 μM cyclopamine (C) starting on day 1.5 of differentiation. Graph below the blot shows quantification of GLI3 immunoblot band intensities (mean±s.e.m., n≥3 independent biological replicates per condition from a total of 8 distinct cell lines). (e) Representative whole 96-well confocal immunofluorescence images of kidney organoid cultures derived from control hPSCs, showing nephron marker expression in these treatment conditions. (f) Quantification of nephron organoids per well in control hPSCs with hedgehog treatments (mean±s.e.m., n=4 independent biological replicates per condition from a total of 4 distinct cell lines; **, p<0.01). (g) Quantification of TUJ1⁺ and (h) SOX2⁺ area in control 96 wells±SAG (mean±s.e.m., n=10 independent biological replicates per condition from a total of 4 distinct cell lines. *p<0.05).

FIGS. 7A-7G. Kinesin-2 knockout organoids express the PKD cystic phenotype. (a) Schematic of cystogenesis assay. (b) Representative phase-contrast images of control, KIF3A^{-/-}, and KIF3B^{-/-} organoids after 10 days in suspension culture. Scale bars, 500 μm. (c) Percentage of cystic organoids in suspension cultures (mean±s.e.m., n≥4 biological replicates per condition from a total of ten distinct cell lines. **, p<0.01; ****, p<0.0001). (d) Representative photographs of whole 6-wells containing organoids grown for several months in suspension culture, with zoom below. Scale bars, 1 cm. (e) 250 ml flask containing a large KIF3B^{-/-} cystic organoid that outgrew the 6-well plates. (f) images showing 3-D confocal reconstruction of the lower edge of a cyst labeled with phospho-histone H3 (pH3) for mitotic cells and LTL for proximal tubular epithelia. Scale bar, 500 μm. (g) Confocal immunofluorescence of cilia (AcTub) and kidney epithelial cell markers in cyst-lining epithelial cells. Scale bar, 25 μm.

FIGS. 8A-8I. Kinesin-2 knockout hPSCs and organoids reveal secretion defects in disease-relevant signaling pathways. (a) Schematic of EV purification and analysis experiment. (b) Representative immunoblots of endogenous ciliary proteins (e.g., KIF3A, IFT88, ARL13B) in supernatants (SN) and lysates (LY) from control and KIF3A^{-/-} hPSCs. β-actin is shown as a loading control. (c) Immunoblots of supernatants showing key components of the PKD (e.g., PC1, PC2) and hedgehog (e.g., GLI2, PTCH1) pathways in these supernatants. β-actin, β-catenin, and flotillin (FLOT1) are shown as loading controls. (d) A panel of non-ciliary EV components in supernatants. (e) PC1 expression in the supernatants of kidney organoids in suspension. Three control and two KIF3A^{-/-} lines are shown. (f) Quantification of PC1 in EVs from control and mutant hPSCs (mean±s.e.m., n≥6 biological replicates per condition, total of 14 distinct cell lines. ***, p<0.001). (g) Quantification of IFT88, PC1, and PTCH1 in EVs from control and mutant hPSCs (n=4 biological replicates, p<0.01). (h) Representative immunoblots of EV from control, PKD1^{-/-}, and kinesin-2 knockout hPSCs, ±SAG treatment, with (i) quantification (mean±s.e.m., n=3 biological replicates per condition, total of 6 distinct cell lines).

FIGS. 9A-9B. Ciliary function directs morphogenesis in complex tissues derived from hPSCs. (a) Schematic of kinesin-2's effects at the cell surface. Left: In normal cells, the cilium generated by kinesin-2 is inhabited by PTCH1 which establishes a protected zone to promote downstream GLI protein processing (relative quantity of GLIF versus GLIR), ultimately resulting in proper modulation of the hedgehog signal and levels of downstream gene expression. Hedgehog and PKD components exit the cell through ciliary EV to further modulate signaling. Right: In kinesin-2 knockout cells, absence of the cilium renders PTCH1 unable to inhibit SMO, resulting in blockade of GLI protein processing and increased activation of the hedgehog signaling pathway. Hedgehog and PKD components are unable to exit the cell. (b) Illustration depicting the role of cilia in tissues and organoids derived from hPSCs. Kinesin-2 activity is responsible for switching the hedgehog signaling between ‘HI’ and ‘LO’ states (white to gray gradient) during differentiation of the pluripotent epiblast into complex tissues (left to right). In differentiated tubular epithelia, kinesin-2 regulates PKD pathway components to restrict expansion into cysts.

FIGS. 10A-10E. Generation of KIF3A^{-/-} and KIF3B^{-/-} hPSC mutants using CRISPR gene editing. (a) Table of gene edited hPSC carrying loss of function mutations in KIF3A or KIF3B generated using CRISPR gene editing. The genetic background, sgRNA used for targeting, and resulting mutation is indicated for each cell line. CATATGGACAAACCGGAAC (SEQ ID NO: 1); TTCGCTGTCGGCCCATGAA (SEQ ID NO: 2); TACACCATGGAAGGAATCCG (SEQ ID NO: 3). (b) Chromatograms of homozygote mutants showing the edited KIF3A Control (SEQ ID NO: 15); WTC11 KIF3A M2 (SEQ ID NO: 16); H9 KIF3A M2 (SEQ ID NO: 17); and H9 KIF3A M4 (SEQ ID NO: 18) or (c) KIF3B regions Control gRNA 1 (SEQ ID NO: 19); WTC11 KIF3B M1 (SEQ ID NO: 20); WTC11 KIF3B M2 (SEQ ID NO: 21); H9 KIF3B M2 (SEQ ID NO: 22); Control gRNA 2 (SEQ ID NO: 23); WTC11 KIF3B M4 (SEQ ID NO: 24); H9 KIF3B M3 (SEQ ID NO: 25). The sgRNA sequence used for targeting is labeled in yellow in the respective control chromatograms. (d) Sanger sequencing chromatogram of compound heterozygote KIF3A WTC11 KIF3A Mutant 1 (SEQ ID NO: 26); Allele 1 (insertion) (SEQ ID NO: 27); H9 KIF3A Mutant 1 (SEQ ID NO: 28); Allele 1 (SEQ ID NO: 29); Allele 2 (SEQ ID NO: 30); H9 KIF3A Mutant 3 (SEQ ID NO: 31); Allele 1 (SEQ ID NO: 32); Allele 2 (SEQ ID NO: 33) or (e) KIF3B mutants and the separated allele sequences after TOPO cloning. Deletions and insertions are indicated in the chromatograms H9 KIF3B Mutant 1 (gRNA 1) (SEQ ID NO: 34); Allele 1 (SEQ ID NO: 35); Allele 2 (SEQ ID NO: 36).

FIGS. 11A-11E. CRISPR gene editing results in loss of KIF3A or KIF3B protein. (a) KIF3A immunoblot showing three distinct control and KIF3A^{-/-} H9 hPSC lines at passages 4 and 17. Cropped areas used in FIG. 1 are shown in bracket box. β-actin was blotted as a loading control. (b) KIF3A immunoblot for one control (C1) and two distinct KIF3A^{-/-} WTC11 hPSC (M1 and M2). GAPDH was blotted as a loading control. (c) KIF3B and β-actin immunoblot for one H9 control (C1) and two KIF3B^{-/-} mutant (M1, M2) hPSC. (d) KIF3B and β-actin immunoblot for three distinct control and four KIF3B^{-/-} WTC11 mutant hPSC. Cropped areas used in FIG. 1 are shown in bracket box. kDa sizes of the protein standards are indicated. (e) Band intensity quantification from immunoblots of the indicated genotype (mean±s.e.m., n≥4 independent biological replicates).

FIGS. 12A-12E. Kinesin-2 is dispensable for hPSC morphology and epiblast spheroid formation. (a) Confocal immunofluorescence images of acetylated α-tubulin (AcTub) and DNA in representative fields of undifferentiated control and KIF3A^{-/-} hPSCs. Orthogonal (top) and volume (bottom) views are shown. (b) Phase contrast images of representative control and KIF3A^{-/-} epiblast spheroids. (c) Quantification of lumen area as percentage of the spheroid area. (d-e) Confocal sections showing immunofluorescence for pluripotency, polarity, and ciliary markers in spheroids. A midbody is seen in a KIF3A^{-/-} spheroid (arrow), but not cilia. Scale bars, 25 μm.

FIGS. 13A-13E. Kinesin-2 knockout hPSCs establish a renewable source of diverse human cell types lacking cilia. (a) Neuroepithelial (AcTub), endodermal (AFP), and mesodermal (SMA) lineages in EBs. Zoom shows magnification of dashed boxed area with AcTub and DNA intensities increased for clarity. Scale bar, 50 μm. (b) Images and (c) quantification of pigmented EBs. Scale bar, 500 μm. Knockout (KO) represent pooled data from both KIF3A^{-/-} and KIF3B^{-/-} EBs (mean±s.e.m., n≥9 independent biological replicates per condition from a total of 8 distinct cell lines). (d) Representative images and (e) quantification of OCT4 and BRY immunofluorescence intensities in hPSCs after treatment with increasing doses of CHIR99021 in mTeSR1 for 48 hours. Each dot represents a single cell. Data are pooled from three separate experiments. bpp, bits per pixel (raw intensity). Scale bars, 50 μm.

FIGS. 14A-14F. Kinesin-2 knockout tumors exhibit differentiation defects. (a) Representative photographs of whole, unfixed growths retrieved from immunodeficient animals at the same time point. Growths are sliced through their centers to reveal the internal surface. (b) A second set of tumors, photographed intact after retrieval from the animals. (c-e) Quantification of tissue subtypes within teratomas as a fraction of total area (mean±s.e.m., n≥5 independent biological replicates per condition from a total of 14 distinct cell lines). (f) Ratio of area occupied by SOX2⁺ cells in TUJ1⁺ patches of teratoma sections (mean±s.e.m., n≥4 independent biological replicates per condition from a total of 6 distinct cell lines; *, p<0.05).

FIGS. 15A-15I. Hedgehog switching is defective in kinesin-2 knockout organoid cultures. (a) Quantification of SOX2+ area per 96-well of kidney organoid cultures (mean±s.e.m., n≥3 independent biological replicates per condition from a total of 11 distinct cell lines. **, p<0.01). (b) Wide-field immunofluorescence images of kidney organoid differentiations in a representative 96-well plate. The three left wells contain control cell lines and the three right wells contain KIF3A^{-/-} cell lines. Zoom of boxed regions is shown below each of the wells. Each kidney organoid contains distal tubular (ECAD), proximal tubular (LTL), and podocyte (NPHS1) epithelial cells. Arrow indicates a cluster of ECAD⁺ cells without proximal tubule and podocyte segments. (c) Heatmap of the expression level of genes differentially expressed as a combined function of KIF3A or KIF3B loss in hPSCs (FDR<0.2). Gene names and labels at right refer to genes associated with enriched gene ontology processes as a function of KIF3B^{-/-} loss in hPSCs in FIG. 5a. (d) Representative GLI3 immunoblot of control, KIF3A^{-/-}, and KIF3B^{-/-} undifferentiated hPSCs, with (e) band intensity quantification (mean±s.e.m., n≥3 independent biological replicates per condition from a total of 11 distinct cell lines). (f) Band intensity quantification of the blot shown in FIG. 5e, comparing cultures on day 0 to day 18. (g) Immunoblot of GLI3 in kidney organoid cultures on days 7, 11, and 18 of differentiation. 3A and 3B indicate KIF3A^{-/-} and KIF3B^{-/-} mutants, respectively. (h) Schematic of organoid microdissection (left), with immunoblot of GLI3 on day 18 of differentiation in lysates of whole wells (w), microdissected organoids (o), or remnant stroma (s). (i) GLI1 immunoblot of the samples shown in FIG. 5e, comparing cultures on day 0 to day 18.

FIGS. 16A-16E. (a) Representative GLI3 immunoblot in organoid cultures on day 18. Blot is from independent biological replicates, compared to the blot shown in FIG. 6d. (b) Individual measurements of GLI3F and (c) GLI3R bands for the sample set quantified in ratiometric form in FIG. 6d. (d) Representative GLI1 blot of these conditions, with (e) quantification of band intensities (mean±s.e.m., n≥3 independent biological replicates per condition from a total of 6 distinct cell lines).

FIGS. 17A-17I. Comparison between kinesin-2 and polycystin knockout mutants. (a) Quantification of organoid area in 12 month old suspension cultures. (b) Representative confocal optical sections showing tubular epithelial markers in cyst-lining epithelia of PKD1 versus KIF3B mutant organoids. (c-e) Representative immunoblot showing PC1 and PC2 levels in KIF3A^{-/-} hPSCs, compared to isogenic controls. b-actin is shown as a loading control. (f) Quantification of band intensities from whole cell lysates of kinesin-2 knockout or control hPSCs (n≥5 independent biological replicates per condition). (g) Immunoblot showing PC2, KIF3A, and GAPDH loading control in kinesin-2 knockout hPSCs, isogenic controls, or PKD2 knockout. (h) Quantification of PC2 band intensities from immunoblots of kinesin-2 knockout hPSCs and isogenic controls (n≥3 independent biological replicates per condition). (i) unprocessed blots for data in panels c-e and g, with dashed outline of cropped areas.

FIGS. 18A-18F. hPSCs release ciliary proteins in EV via kinesin-2. (a) Quantification of protein fraction in EV (SN), compared to whole cell lysates (LY), as a percentage of total protein (SN+LY), using BCA assay (n=four distinct hPSC lines, plotted separately). (b) Representative immunoblot showing IFT88 levels in LY vs. SN. Total protein load is indicated in μg above each lane. (c) Quantification of IFT88 band intensities per lane at increasing total protein loads in SN and LY (n=4 independent biological replicates from a total of 4 distinct cell lines). (d-e) Representative immunoblots and (f) band intensity quantifications of key ciliary proteins in EV, in KIF3B^{-/-} hPSCs compared to isogenic controls (mean±s.e.m., n≥2 independent biological replicates from a total of 4 distinct cell lines).

FIGS. 19A-19D. Kinesin-2 knockout hPSCs exhibit secretion defects with normal cytoplasmic expression. (a) Representative immunoblots of endogenous PC1 or (b) PC2 proteins in supernatants (SN) and lysates (LY) from control, PKD1^{-/-}, PKD2^{-/-}, and KIF3A^{-/-} hPSCs, with FLOT1 loading control. PC2ₜₑₜᵣₐ refers to the tetrameric form of PC2. (c) Silver stain of whole supernatants from two control and three KIF3A^{-/-} hPSC lines. No consistent differences in banding pattern are observed between the two groups. (d) Uncropped immunoblots for PTCH1 and IFT88 in EV, from FIGS. 8b and 8c, respectively.

### DETAILED DESCRIPTION

Cilia are antenna-like organelles associated with a spectrum of human disease states. The functions of cilia remain incompletely understood, particularly in humans where cellular models do not recapitulate complex disease. The present disclosure describes that human pluripotent stem cells lacking a kinesin-2 subunit, either KIF3A or KIF3B, generated by CRISPR gene editing, establish a general tool for the study of ciliary function in tissue morphogenesis. Both KIF3A^{-/-} and KIF3B^{-/-} human pluripotent stem cells (hPSCs) lack cilia, yet remain self-renewing and pluripotent, enabling efficient derivation of diverse somatic cells. Derived tissues and organoids express multi-lineage phenotypes modeling the ciliopathy spectrum, including neuronal and nephron differentiation defects, and polycystic kidney disease. Modular culture conditions and biochemical analysis reveal that human cilia mediate a critical switch in hedgehog signaling during organoid differentiation, and constitutively release extracellular vesicles containing signaling molecules associated with these phenotypes. KIF3A^{-/-} and KIF3B^{-/-} hPSCs thus establish a human genetic system linking complex ciliary phenotypes with endogenous mechanisms, for therapeutics discovery and regenerative applications.

Structurally, eukaryotic cilia comprise a bundle of parallel, axonemal microtubules anchored to a basal body and encased in a sheath of plasma membrane and can be present individually (primary cilia) or in multi-ciliated arrays. A conserved pathway called intraflagellar transport (IFT) facilitates the ciliary import and export of specific molecular cargoes along the axonemal microtubules. Kinesin-2 is a family of motor proteins such as KIF3A and KIF3B that play important roles in IFT and ciliogenesis. Mutations in over fifty genes associated with ciliary structure, transport, or function cause a spectrum of ciliopathy syndromes, such as nephronophthisis (NPHP), Joubert syndrome, and PKD. Cilia are absent in mitotic cells and many tumors and can act as a brake on cell division. The presence or absence of cilia can also affect the development of medulloblastomas and basal cell carcinomas. Cilia have therefore emerged as an important therapeutic target for diverse disease states.

A major barrier to understanding ciliary function is the absence of cellular models that accurately reconstitute complex human phenotypes and disease mechanisms. In mice, genetic disruption of IFT results in complete loss of cilia and early embryonic lethality, precluding further analysis. Conditional or hypomorphic mutations can produce tissue-specific phenotypes, such as retinal degeneration and cystic kidney disease, but cilia are only partially ablated in these models, which do not fully genocopy or phenocopy humans. At the other end of the spectrum, primary or immortalized cells with ciliopathy mutations are rare, lineage-restricted, heterogenous, and incapable of recapitulating complex tissue phenotypes. In contrast, human pluripotent stem cells (hPSCs), including embryonic stem cells and induced pluripotent stem cells, possess an extensive capacity for complex tissue differentiation (Thomson, J. A. et al. Embryonic stem cell lines derived from human blastocysts. Science 282, 1145-1147 (1998); Takahashi, K. et al. Induction of pluripotent stem cells from adult human fibroblasts by defined factors. Cell 131, 861-872 (2007)). Undifferentiated hPSCs possess primary cilia and can exhibit defects in ciliary trafficking, number, or length linked to disease (Kiprilov, E. N. et al. Human embryonic stem cells in culture possess primary cilia with hedgehog signaling machinery. J Cell Biol 180, 897-904 (2008). Freedman, B. S. et al. Reduced ciliary polycystin-2 in induced pluripotent stem cells from polycystic kidney disease patients with PKD1 mutations. J Am Soc Nephrol 24, 1571-1586 (2013)). Gene editing methods, such as the Cas9/CRISPR (clustered regularly interspaced short palindromic repeats) system, further enable elegant genetic models featuring mutant and control hPSCs of identical genetic background (Jinek, M. et al. A programmable dual-RNA-guided DNA endonuclease in adaptive bacterial immunity. Science 337, 816-821 (2012); Mali, P. et al. RNA-guided human genome engineering via Cas9. Science 339, 823-826 (2013); Freedman, B. S. et al. Modelling kidney disease with CRISPR-mutant kidney organoids derived from human pluripotent epiblast spheroids. Nat Commun 6, 8715 (2015)).

As described in more detail below, the inventors developed gene-edited human pluripotent stem cells (hPSCs) lacking one or both kinesin-2 subunits. The inventors have further used the gene-edited hPSCs to generate organoids lacking one or both kinesin-2 subunits, hPSCs or organoids lacking one or both kinesin-2 subunits (i.e., KIF3A or KIF3B) that lack cilia. To this point, there has been an absence of cellular models that accurately reconstitute human phenotypes and disease mechanisms due to a defect in ciliary function. This work provides genetically modified hPSCs and genetically modified organoids, and methods for making the same, that lack cilia. These novel model systems can be used to recapitulate hallmark features of ciliopathies and reveal molecular functions associated with functioning cilia.

### Compositions of the Claimed Embodiments

In accordance with the foregoing, in one aspect the disclosure provides isolated genetically modified pluripotent stem cells (PSCs) comprising a disruption in a gene encoding at least one subunit of kinesin-2, wherein the genetic modification results in PSC lacking a ciliary structure. In some embodiments, the genetic modification comprises disruption of KIF3A and reduced expression of KIF3A protein, wherein reduced expression of KIF3A protein results in PSCs that lack ciliary structures. In other embodiments, the genetic modification comprises disruption of KIF3B and reduced expression of KIF3B protein, wherein reduced expression of KIF3B protein results in PSCs lacking ciliary structures. In still other embodiments, the genetic modification comprises disruption of KIF3A and KIF3B and reduced expression of KIF3A and KIF3B protein, wherein reduced expression of KIF3A and KIF3B protein results in PSCs lacking ciliary structures.

As used herein, the term “stem cell” refers to master cells which can unlimitedly regenerate cells so as to form specialized cells of tissues and organs. The stem cells are developable multipotent or pluripotent cells. The stem cell can be cell-divided into two daughter stem cells, or one daughter stem cell and one transit cell, and the cells subsequently proliferate to mature and complete type of cells or tissues. These stem cells may be classified by various methods. One of the most commonly used methods depends on the differentiation capability of the stem cells. According to the method, the stem cells may be classified into pluripotent stem cells that are differentiable into three-germ layer cells, multipotent stem cells that are limitedly differentiable into a specific germ layer or more, and unipotent stem cells that are differentiable only into a specific germ layer. In some embodiments, the adult stem cell can be a multipotent stem cell or a unipotent stem cell.

The term “pluripotent stem cells” (PSC) as used herein refers to stem cells having pluripotency, which are capable of differentiating into all three germ layers constituting a living body. In some embodiments, the PSC can be a human PSC (pPSC). In some embodiments, the PSC is an embryonic stem cell. In some embodiments, the PSC is an induced PSC. In some embodiments, the genetically modified PSC can include but is not limited to a human PSC (hPSC), a human embryonic stem cell (hESC), and an induced PCS (iPSC).

As used herein, “embryonic stem cells” or “ESCs” refers to a pluripotent cell or population of pluripotent cells derived from an inner cell mass of a blastocyst. As used herein, “induced pluripotent stem cells” or “iPSCs” refers to a pluripotent cell or population of pluripotent cells that can vary with respect to their differentiated somatic cell of origin, that can vary with respect to a specific set of potency-determining factors and that can vary with respect to culture conditions used to isolate them, but nonetheless are substantially genetically identical to their respective differentiated somatic cell of origin and display characteristics similar to higher potency cells, such as ESCs.

The term “differentiation,” as used herein, refers to a process by which cells become specialized in structure or function during division, proliferation and growth of cells, that is, change of morphology or function of cells so that cells, tissues, etc. of an organism perform given works thereof. Generally, it is a process of a relatively simple system being separated into two or more qualitatively different partial systems. The differentiation refers to a state in which parts of a certain bio-system, that have been homogeneous at first, become qualitatively distinguished from one another, or as the result thereof, they become divided into qualitatively distinguishable parts or partial systems such as, for example, in terms of ontogenesis an egg, which was homogeneous at first, becomes distinguished into head, body, etc. or cells as myocytes, neurocytes, etc. become distinguished from one another.

The term “embryonic body (EB),” as used herein, refers to an aggregate formed by inducing differentiation of the pluripotent stem cells. The embryonic body may be generated when the pluripotent stem cells are cultured in a suspension state without feeders in an embryonic stem cell medium free from a basic fibroblast growth factor (bFGF). The embryonic body prepared by the above method has been reported to be able to differentiate into all cells necessary for formation of an individual from endoderm, mesoderm, and ectoderm, and this corresponds to one of the in vitro methods that prove pluripotency of the pluripotent stem cells.

In some embodiments, the isolated PSC can be expanded to comprise an isolated genetically modified PSC cell line according to any method well-known to one of ordinary skill in the art.

As used herein, “isolated” refers to a cell or a population of cells which has been separated from at least some components of its natural environment.

As used herein, the term “cilia” refers to hair-like membrane-enclosed tubular structures that extend from the cell's epithelial surface into, for example, a lumen that is in contact with the environment. See e.g., Lodish et al., 2000, Molecular Cell Biology, 4th edition.

As used herein, a “ciliary structure” refers to any structure comprising the primary cilium. The core structure of a primary cilia is composed of microtubule bundles (i.e., ciliary axoneme) extending from the basal body. Cilia can be present individually (i.e., primary cilia) or in multi-ciliated arrays.

In some embodiments, a ciliary structure can be a ciliary axoneme. As used herein a ciliary axoneme is a short, thin, elongated structure at the apical surface of cells enriched for ciliary markers such as acetylated alpha tubulin and ARL13B. The axoneme is anchored in the cell by a basal body and comprises either a 9+0 or a 9+2 arrangement of microtubules. Primary cilia comprise a ciliary membrane surrounding a doublet of microtubules (e.g., 9+0). Motile cilia comprise a ciliary membrane surrounding a doublet of microtubules, further surrounding singlet microtubules (e.g., 9+2). In motile cilia, tubules work with a network of dynein arms and protein spokes to produce movement.

In some embodiments, 50%, 60%, 70%, 80%, or 90% of the genetically modified PSCs lack cilia (e.g., primary cilia, multi-ciliated arrays, or primary cilia and multi-ciliated arrays). In other embodiments, at least 90% of the genetically modified PSCs lack cilia (e.g., primary cilia, multi-ciliated arrays, or primary cilia and multi-ciliated arrays).

In other embodiments, 90%, 91%, 92%, 93%, 94%, 95%, 96%, 97%, 98%, or 99% of the genetically modified PSCs lack cilia (e.g., primary cilia, multi-ciliated arrays, or primary cilia and multi-ciliated arrays). In still other embodiments, 100% of the genetically modified PSCs lack cilia (e.g., primary cilia, multi-ciliated arrays, or primary cilia and multi-ciliated arrays).

In some embodiments, 50%, 60%, 70%, 80%, or 90% of the genetically modified PSCs lack ciliary axonemes. In other embodiments, at least 90% of the genetically modified PSCs lack ciliary axonemes. In other embodiments, 90%, 91%, 92%, 93%, 94%, 95%, 96%, 97%, 98%, or 99% of the genetically modified PSCs lack ciliary axonemes. In still other embodiments, 100% of the genetically modified PSCs lack ciliary axonemes.

In some embodiments, 50%, 60%, 70%, 80%, or 90% of the genetically modified PSCs lack the ciliary protein ARL13B⁺. In other embodiments, at least 90% of the genetically modified PSCs lack the ciliary protein ARL13B⁺. In other embodiments, 90%, 91%, 92%, 93%, 94%, 95%, 96%, 97%, 98%, or 99% of the genetically modified PSCs lack the ciliary protein ARL13B⁺. In still other embodiments, 100% of the genetically modified PSCs lack the ciliary protein ARL13B⁺.

In some embodiments, 50%, 60%, 70%, 80%, or 90% of the genetically modified PSCs lack the ciliary marker acetylated α-tubulin⁺. In other embodiments, at least 90% of the genetically modified PSCs lack the ciliary marker acetylated α-tubulin⁺. In other embodiments, 90%, 91%, 92%, 93%, 94%, 95%, 96%, 97%, 98%, or 99% of the genetically modified PSCs lack the ciliary marker acetylated α-tubulin⁺. In still other embodiments, 100% of the genetically modified PSCs lack the ciliary marker acetylated α-tubulin⁺.

In some embodiments, 50%, 60%, 70%, 80%, or 90% of the genetically modified PSCs lack the ciliary protein ARL13B⁺ and the ciliary marker acetylated α-tubulin⁺. In other embodiments, at least 90% of the genetically modified PSCs lack the ciliary protein ARL13B⁺ and the ciliary marker acetylated α-tubulin⁺. In other embodiments, 90%, 91%, 92%, 93%, 94%, 95%, 96%, 97%, 98%, or 99% of the genetically modified PSCs lack the ciliary protein ARL13B⁺ and the ciliary marker acetylated α-tubulin⁺. In still other embodiments, 100% of the genetically modified PSCs lack the ciliary protein ARL13B⁺ and the ciliary marker acetylated α-tubulin⁺.

In some embodiments, cilia (e.g., primary cilia, multi-ciliated arrays, or primary cilia and multi-ciliated arrays) on the genetically modified PSCs are reduced by 50%, 60%, 70%, 80%, or 90% compared to unmodified PSCs. In other embodiments, the cilia (e.g., primary cilia, multi-ciliated arrays, or primary cilia and multi-ciliated arrays) on the genetically modified PSCs are reduced by at least 90% compared to unmodified PSCs. In still other embodiments, the cilia (e.g., primary cilia, multi-ciliated arrays, or primary cilia and multi-ciliated arrays) on the genetically modified PSCs are reduced by 90%, 91%, 92%, 93%, 94%, 95%, 96%, 97%, 98%, or 99% compared to unmodified PSCs. In still other embodiments, the cilia (e.g., primary cilia, multi-ciliated arrays, or primary cilia and multi-ciliated arrays) on the genetically modified PSCs are reduced by 100% compared to unmodified PSCs.

In some embodiments, reduced expression of KIF3A results in 50%, 60%, 70%, 80%, or 90% of the genetically modified PSCs lacking cilia (e.g., primary cilia, multi-ciliated arrays, or primary cilia and multi-ciliated arrays). In other embodiments, reduced expression of KIF3A results in at least 90% of the genetically modified PSCs lacking cilia (e.g., primary cilia, multi-ciliated arrays, or primary cilia and multi-ciliated arrays). In other embodiments, reduced expression of KIF3A results in 90%, 91%, 92%, 93%, 94%, 95%, 96%, 97%, 98%, or 99% of the genetically modified PSCs lacking cilia (e.g., primary cilia, multi-ciliated arrays, or primary cilia and multi-ciliated arrays). In still other embodiments, reduced expression of KIF3A results in 100% of the genetically modified PSCs lacking cilia (e.g., primary cilia, multi-ciliated arrays, or primary cilia and multi-ciliated arrays).

In some embodiments, reduced expression of KIF3A results in 50%, 60%, 70%, 80%, or 90% of the genetically modified PSCs lacking ciliary axonemes. In other embodiments, reduced expression of KIF3A results in at least 90% of the genetically modified PSCs lacking ciliary axonemes. In other embodiments, reduced expression of KIF3A results in 90%, 91%, 92%, 93%, 94%, 95%, 96%, 97%, 98%, or 99% of the genetically modified PSCs lacking ciliary axonemes. In still other embodiments, reduced expression of KIF3A results in 100% of the genetically modified PSCs lacking ciliary axonemes.

In some embodiments, reduced expression of KIF3A results in 50%, 60%, 70%, 80%, or 90% of the genetically modified PSCs lacking the ciliary protein ARL13B⁺. In other embodiments, reduced expression of KIF3A results in at least 90% of the genetically modified PSCs lacking the ciliary protein ARL13B⁺. In other embodiments, reduced expression of KIF3A results in 90%, 91%, 92%, 93%, 94%, 95%, 96%, 97%, 98%, or 99% of the genetically modified PSCs lacking the ciliary protein ARL13B⁺. In still other embodiments, reduced expression of KIF3A results in 100% of the genetically modified PSCs lacking the ciliary protein ARL13B⁺.

In some embodiments, reduced expression of KIF3A results in 50%, 60%, 70%, 80%, or 90% of the genetically modified PSCs lacking the ciliary marker acetylated α-tubulin⁺. In other embodiments, reduced expression of KIF3A results in at least 90% of the genetically modified PSCs lacking the ciliary marker acetylated α-tubulin⁺. In other embodiments, reduced expression of KIF3A results in 90%, 91%, 92%, 93%, 94%, 95%, 96%, 97%, 98%, or 99% of the genetically modified PSCs lacking the ciliary marker acetylated α-tubulin⁺. In still other embodiments, reduced expression of KIF3A results in 100% of the genetically modified PSCs lacking the ciliary marker acetylated α-tubulin⁺.

In some embodiments, reduced expression of KIF3A results in 50%, 60%, 70%, 80%, or 90% of the genetically modified PSCs lacking the ciliary protein ARL13B⁺ and the ciliary marker acetylated α-tubulin⁺. In other embodiments, reduced expression of KIF3A results in at least 90% of the genetically modified PSCs lacking the ciliary protein ARL13B⁺ and the ciliary marker acetylated α-tubulin⁺. In other embodiments, reduced expression of KIF3A results in 90%, 91%, 92%, 93%, 94%, 95%, 96%, 97%, 98%, or 99% of the genetically modified PSCs lacking the ciliary protein ARL13B⁺ and the ciliary marker acetylated α-tubulin⁺. In still other embodiments, reduced expression of KIF3A results in 100% of the genetically modified PSCs lacking the ciliary protein ARL13B⁺ and the ciliary marker acetylated α-tubulin⁺.

In some embodiments, reduced expression of KIF3A results in cilia (e.g., primary cilia, multi-ciliated arrays, or primary cilia and multi-ciliated arrays) on the genetically modified PSCs being reduced by 50%, 60%, 70%, 80%, or 90% compared to unmodified PSCs. In other embodiments, reduced expression of KIF3A results in the cilia (e.g., primary cilia, multi-ciliated arrays, or primary cilia and multi-ciliated arrays) on the genetically modified PSCs being reduced by at least 90% compared to unmodified PSCs. In still other embodiments, reduced expression of KIF3A results in the cilia (e.g., primary cilia, multi-ciliated arrays, or primary cilia and multi-ciliated arrays) on the genetically modified PSCs being reduced by 90%, 91%, 92%, 93%, 94%, 95%, 96%, 97%, 98%, or 99% compared to unmodified PSCs. In still other embodiments, reduced expression of KIF3A results in the cilia (e.g., primary cilia, multi-ciliated arrays, or primary cilia and multi-ciliated arrays) on the genetically modified PSCs being reduced by 100% compared to unmodified PSCs.

In some embodiments, reduced expression of KIF3B results in 50%, 60%, 70%, 80%, or 90% of the genetically modified PSCs lacking cilia (e.g., primary cilia, multi-ciliated arrays, or primary cilia and multi-ciliated arrays). In other embodiments, reduced expression of KIF3B results in at least 90% of the genetically modified PSCs lacking cilia (e.g., primary cilia, multi-ciliated arrays, or primary cilia and multi-ciliated arrays). In other embodiments, reduced expression of KIF3B results in 90%, 91%, 92%, 93%, 94%, 95%, 96%, 97%, 98%, or 99% of the genetically modified PSCs lacking cilia (e.g., primary cilia, multi-ciliated arrays, or primary cilia and multi-ciliated arrays). In still other embodiments, reduced expression of KIF3B results in 100% of the genetically modified PSCs lacking cilia (e.g., primary cilia, multi-ciliated arrays, or primary cilia and multi-ciliated arrays).

In some embodiments, reduced expression of KIF3B results in 50%, 60%, 70%, 80%, or 90% of the genetically modified PSCs lacking ciliary axonemes. In other embodiments, reduced expression of KIF3B results in at least 90% of the genetically modified PSCs lacking ciliary axonemes. In other embodiments, reduced expression of KIF3B results in 90%, 91%, 92%, 93%, 94%, 95%, 96%, 97%, 98%, or 99% of the genetically modified PSCs lacking ciliary axonemes. In still other embodiments, reduced expression of KIF3B results in 100% of the genetically modified PSCs lacking ciliary axonemes.

In some embodiments, reduced expression of KIF3B results in 50%, 60%, 70%, 80%, or 90% of the genetically modified PSCs lacking the ciliary protein ARL13B⁺. In other embodiments, reduced expression of KIF3B results in at least 90% of the genetically modified PSCs lacking the ciliary protein ARL13B⁺. In other embodiments, reduced expression of KIF3B results in 90%, 91%, 92%, 93%, 94%, 95%, 96%, 97%, 98%, or 99% of the genetically modified PSCs lacking the ciliary protein ARL13B⁺. In still other embodiments, reduced expression of KIF3B results in 100% of the genetically modified PSCs lacking the ciliary protein ARL13B⁺.

In some embodiments, reduced expression of KIF3B results in 50%, 60%, 70%, 80%, or 90% of the genetically modified PSCs lacking the ciliary marker acetylated α-tubulin⁺. In other embodiments, reduced expression of KIF3B results in at least 90% of the genetically modified PSCs lacking the ciliary marker acetylated α-tubulin⁺. In other embodiments, reduced expression of KIF3B results in 90%, 91%, 92%, 93%, 94%, 95%, 96%, 97%, 98%, or 99% of the genetically modified PSCs lacking the ciliary marker acetylated α-tubulin⁺. In still other embodiments, reduced expression of KIF3B results in 100% of the genetically modified PSCs lacking the ciliary marker acetylated α-tubulin⁺.

In some embodiments, reduced expression of KIF3B results in 50%, 60%, 70%, 80%, or 90% of the genetically modified PSCs lacking the ciliary protein ARL13B⁺ and the ciliary marker acetylated α-tubulin⁺. In other embodiments, reduced expression of KIF3B results in at least 90% of the genetically modified PSCs lacking the ciliary protein ARL13B⁺ and the ciliary marker acetylated α-tubulin⁺. In other embodiments, reduced expression of KIF3B results in 90%, 91%, 92%, 93%, 94%, 95%, 96%, 97%, 98%, or 99% of the genetically modified PSCs lacking the ciliary protein ARL13B⁺ and the ciliary marker acetylated α-tubulin⁺. In still other embodiments, reduced expression of KIF3B results in 100% of the genetically modified PSCs lacking the ciliary protein ARL13B⁺ and the ciliary marker acetylated α-tubulin⁺.

In some embodiments, reduced expression of KIF3B results in cilia (e.g., primary cilia, multi-ciliated arrays, or primary cilia and multi-ciliated arrays) on the genetically modified PSCs being reduced by 50%, 60%, 70%, 80%, or 90% compared to unmodified PSCs. In other embodiments, reduced expression of KIF3A results in the cilia (e.g., primary cilia, multi-ciliated arrays, or primary cilia and multi-ciliated arrays) on the genetically modified PSCs being reduced by at least 90% compared to unmodified PSCs. In still other embodiments, reduced expression of KIF3A results in the cilia (e.g., primary cilia, multi-ciliated arrays, or primary cilia and multi-ciliated arrays) on the genetically modified PSCs being reduced by 90%, 91%, 92%, 93%, 94%, 95%, 96%, 97%, 98%, or 99% compared to unmodified PSCs. In still other embodiments, reduced expression of KIF3A results in the cilia (e.g., primary cilia, multi-ciliated arrays, or primary cilia and multi-ciliated arrays) on the genetically modified PSCs being reduced by 100% compared to unmodified PSCs.

In some embodiments, reduced expression of KIF3A and KIF3B results in 50%, 60%, 70%, 80%, or 90% of the genetically modified PSCs lacking cilia (e.g., primary cilia, multi-ciliated arrays, or primary cilia and multi-ciliated arrays). In other embodiments, reduced expression of KIF3A and KIF3B results in at least 90% of the genetically modified PSCs lacking cilia (e.g., primary cilia, multi-ciliated arrays, or primary cilia and multi-ciliated arrays). In other embodiments, reduced expression of KIF3A and KIF3B results in 90%, 91%, 92%, 93%, 94%, 95%, 96%, 97%, 98%, or 99% of the genetically modified PSCs lacking cilia (e.g., primary cilia, multi-ciliated arrays, or primary cilia and multi-ciliated arrays). In still other embodiments, reduced expression of KIF3A and KIF3B results in 100% of the genetically modified PSCs lacking cilia (e.g., primary cilia, multi-ciliated arrays, or primary cilia and multi-ciliated arrays).

In some embodiments, reduced expression of KIF3A and KIF3B results in 50%, 60%, 70%, 80%, or 90% of the genetically modified PSCs lacking ciliary axonemes. In other embodiments, reduced expression of KIF3A and KIF3B results in at least 90% of the genetically modified PSCs lacking ciliary axonemes. In other embodiments, reduced expression of KIF3A and KIF3B results in 90%, 91%, 92%, 93%, 94%, 95%, 96%, 97%, 98%, or 99% of the genetically modified PSCs lacking ciliary axonemes. In still other embodiments, reduced expression of KIF3A and KIF3B result in 100% of the genetically modified PSCs lacking ciliary axonemes.

In some embodiments, reduced expression of KIF3A and KIF3B results in 50%, 60%, 70%, 80%, or 90% of the genetically modified PSCs lacking the ciliary protein ARL13B⁺. In other embodiments, reduced expression of KIF3A and KIF3B results in at least 90% of the genetically modified PSCs lacking the ciliary protein ARL13B⁺. In other embodiments, reduced expression of KIF3A and KIF3B results in 90%, 91%, 92%, 93%, 94%, 95%, 96%, 97%, 98%, or 99% of the genetically modified PSCs lacking the ciliary protein ARL13B⁺. In still other embodiments, reduced expression of KIF3A and KIF3B results in 100% of the genetically modified PSCs lacking the ciliary protein ARL13B⁺.

In some embodiments, reduced expression of KIF3A and KIF3B results in 50%, 60%, 70%, 80%, or 90% of the genetically modified PSCs lacking the ciliary marker acetylated α-tubulin⁺. In other embodiments, reduced expression of KIF3A and KIF3B results in at least 90% of the genetically modified PSCs lacking the ciliary marker acetylated α-tubulin⁺. In other embodiments, reduced expression of KIF3A and KIF3B results in 90%, 91%, 92%, 93%, 94%, 95%, 96%, 97%, 98%, or 99% of the genetically modified PSCs lacking the ciliary marker acetylated α-tubulin⁺. In still other embodiments, reduced expression of KIF3A and KIF3B results in 100% of the genetically modified PSCs lacking the ciliary marker acetylated α-tubulin⁺.

In some embodiments, reduced expression of KIF3A and KIF3B results in 50%, 60%, 70%, 80%, or 90% of the genetically modified PSCs lacking the ciliary protein ARL13B⁺ and the ciliary marker acetylated α-tubulin⁺. In other embodiments, reduced expression of KIF3A and KIF3B results in at least 90% of the genetically modified PSCs lacking the ciliary protein ARL13B⁺ and the ciliary marker acetylated α-tubulin⁺. In other embodiments, reduced expression of KIF3A and KIF3B results in 90%, 91%, 92%, 93%, 94%, 95%, 96%, 97%, 98%, or 99% of the genetically modified PSCs lacking the ciliary protein ARL13B⁺ and the ciliary marker acetylated α-tubulin⁺. In still other embodiments, reduced expression of KIF3A and KIF3B results in 100% of the genetically modified PSCs lacking the ciliary protein ARL13B⁺ and the ciliary marker acetylated α-tubulin⁺.

In some embodiments, reduced expression of KIF3A and KIF3B results in cilia (e.g., primary cilia, multi-ciliated arrays, or primary cilia and multi-ciliated arrays) on the genetically modified PSCs being reduced by 50%, 60%, 70%, 80%, or 90% compared to unmodified PSCs. In other embodiments, reduced expression of KIF3A and KIF3B results in the cilia (e.g., primary cilia, multi-ciliated arrays, or primary cilia and multi-ciliated arrays) on the genetically modified PSCs being reduced by at least 90% compared to unmodified PSCs. In still other embodiments, reduced expression of KIF3A and KIF3B results in the cilia (e.g., primary cilia, multi-ciliated arrays, or primary cilia and multi-ciliated arrays) on the genetically modified PSCs being reduced by 90%, 91%, 92%, 93%, 94%, 95%, 96%, 97%, 98%, or 99% compared to unmodified PSCs. In still other embodiments, reduced expression of KIF3A and KIF3B results in the cilia (e.g., primary cilia, multi-ciliated arrays, or primary cilia and multi-ciliated arrays) on the genetically modified PSCs being reduced by 100% compared to unmodified PSCs.

As used herein, Kinesin-2 is a member of the kinesin superfamily of motor proteins found in eukaryotic cells. Kinesins are enzymes that have a core kinesin motor domain for ATP-dependent movement along the surface of microtubules. Specifically, kinesin-2 comprises the kinesin-like proteins, KIF3A and KIF3B, which comprise a two-headed anterograde motor that has an important role in intraflagellar transport and ciliogenesis. KIF3A is encoded by the KIF3A gene. The KIF3A Ensembl gene identifier is ENSG00000131437. KIF3B is encoded by the KIF3B gene. The KIF3B Ensembl gene identifier is ENSG00000101350. KIF3A and KIF3B form a heterodimer that is membrane-bound and has ATPase activity.

As used herein, “disrupting a gene,” “disrupting a gene function,” or any grammatically similar variants (e.g., modifying a gene or modifying a gene function) refers to editing the gene using any gene editing techniques well-known to one with ordinary skill in the art. The edited genes comprise a mutation that eliminates or reduces functional expression of the protein encoded by the gene. In some embodiments, the mutation can be a small insertion or deletion that results in a frame shift, resulting in a nonsense mutation.

As used herein, the term “reduced expression” refers to any reduction in the expression of the KIF3A protein, the KIF3B protein, or the KIF3A and KIF3B proteins compared to an unmodified control cell. The term “reduced functional expression” refers to any reduction in the expression of functional KIF3A protein, functional KIF3B protein, or functional KIF3B protein and functional KIF3A protein compared to an unmodified control cell. The term reduced can also refer to a reduction in the percentage of cells in a population of cells that express an KIF3A, KIF3B, or the KIF3A and KIF3B proteins compared to an unmodified control cell. In other embodiments, the term reduced can also refer to a reduction in the percentage of cells in a population of cells that express a functional KIF3A protein, a functional KIF3B protein, or functional KIF3A and KIF3B proteins compared to an unmodified control cell. As used herein, such a reduction can be up to 5%, 10%, 20%, 30%, 40%, 50%, 60%, 70%, 80%, 90%, 95%, or up to 100%. Accordingly, the term “reduced” encompasses both a partial knockdown and a complete knockdown of KIF3A, KIF3B, or KIF3A and KIF3B protein.

In accordance with the foregoing, in one aspect the disclosure provides a genetically modified cell descended from the genetically modified pluripotent stem cell (PSC), as described above.

As used herein, cells descended from PSCs include progeny cells that can further undergo differentiation into cell types that collectively exhibit characteristics associated with cell lineages from the three germ layers (i.e., endoderm, mesoderm, and ectoderm). In some embodiments, the cell types derived from the endoderm include but are not limited to lung cells, thyroid cells, or pancreatic cells. In some embodiments, the cell types derived from the mesoderm include but are not limited to, cardiac muscle cells, skeletal muscle cells, tubules cells of the kidney, red blood cells, or smooth muscle cells. In other embodiments, the cell types derived from the ectoderm include but are not limited to, skin cells of the epidermis, neuron cells, or pigment cells.

In accordance with the foregoing, in one aspect the disclosure provides an isolated genetically modified organoid derived from the genetically modified pluripotent stem cells (PSCs), as described above.

As used herein, the term “organoid” refers to an in vitro three-dimensional multicellular construct developed from the genetically modified PSCs described. Organoids contain multiple cell types of their in vivo counter-part and organize similarly to the primary tissue. In some embodiments, the organoid culture system includes an organoid culture medium and an extracellular matrix or extracellular matrix substitute.

A phenotypic organoid model may be generated for any type of organoid including, but not limited to, kidney organoids, gut organoids, liver organoids, pancreatic organoids, ovary organoids, brain organoids, and cancer organoids. The organoids generated in accordance with the methods described herein may act as a model for a phenotype related to a disease or condition. Each type of organoid may be generated from differentiation of one or more hPSC cell line described above, and each cell line may require different optimal differentiation conditions to form the phenotypic organoid.

In some embodiments, the genetically modified organoid comprises a neuronal organoid. In some embodiments, the neuronal organoid comprises at least one neuronal structure. In some embodiments, at least 5%, at least 10%, at least 20%, at least 30%, at least 40%, at least 50%, at least 60%, at least 70%, at least 80%, at least 90%, at least 95%, or up to 100% of neuronal organoids comprise at least one neuronal structure. In other embodiments, at least 50% at least 55%, at least 60%, at least 65%, at least 70, at least 75%, at least 80%, at least 85%, at least 90%, at least 95%, or up to 100% of neuronal organoids comprise at least one neuronal structure. In still other embodiments, at least 90%, at least 91%, at least 92%, at least 93%, at least 94%, at least 95%, at least 96%, at least 97%, at least 98%, at least 99%, or up to 100% of neuronal organoids comprise at least one neuronal structure. Neuronal structures can include, but are not limited to, any neuronal structures well-known to one of ordinary skill in the art.

In some embodiments, the neuronal structures comprise at least one axon-like rosette morphology. In some embodiments, at least 5%, at least 10%, at least 20%, at least 30%, at least 40%, at least 50%, at least 60%, at least 70%, at least 80%, at least 90%, at least 95%, or up to 100% of neuronal organoids comprise at least one axon-like rosette morphology. In other embodiments, at least 50% at least 55%, at least 60%, at least 65%, at least 70, at least 75%, at least 80%, at least 85%, at least 90%, at least 95%, or up to 100% of neuronal organoids comprise at least one axon-like rosette morphology. In still other embodiments, at least 90%, at least 91%, at least 92%, at least 93%, at least 94%, at least 95%, at least 96%, at least 97%, at least 98%, at least 99%, or up to 100% of neuronal organoids comprise at least one axon-like rosette morphology.

In some embodiments, the neuronal organoid comprises at least one neuronal marker. In some embodiments, at least 5%, at least 10%, at least 20%, at least 30%, at least 40%, at least 50%, at least 60%, at least 70%, at least 80%, at least 90%, at least 95%, or up to 100% of neuronal organoids comprise at least one neuronal marker. In other embodiments, at least 50% at least 55%, at least 60%, at least 65%, at least 70, at least 75%, at least 80%, at least 85%, at least 90%, at least 95%, or up to 100% of neuronal organoids comprise at least one neuronal marker. In still other embodiments, at least 90%, at least 91%, at least 92%, at least 93%, at least 94%, at least 95%, at least 96%, at least 97%, at least 98%, at least 99%, or up to 100% of neuronal organoids comprise at least one neuronal marker. Neuronal markers can include, but are not limited to, any neuronal gene expression markers well-known to one of ordinary skill in the art.

In some embodiments, the neuronal structures comprise at least the gene expression marker SRY-box 2 (SOX2). In some embodiments, at least 5%, at least 10%, at least 20%, at least 30%, at least 40%, at least 50%, at least 60%, at least 70%, at least 80%, at least 90%, at least 95%, or up to 100% of neuronal organoids comprise at least the gene expression marker SOX2. In other embodiments, at least 50% at least 55%, at least 60%, at least 65%, at least 70, at least 75%, at least 80%, at least 85%, at least 90%, at least 95%, or up to 100% of neuronal organoids comprise at least the gene expression marker SOX2. In still other embodiments, at least 90%, at least 91%, at least 92%, at least 93%, at least 94%, at least 95%, at least 96%, at least 97%, at least 98%, at least 99%, or up to 100% of neuronal organoids comprise at least the gene expression marker SOX2.

In some embodiments, the neuronal structures comprise at least the gene expression marker class III beta-tubulin (TuJ1). In some embodiments, at least 5%, at least 10%, at least 20%, at least 30%, at least 40%, at least 50%, at least 60%, at least 70%, at least 80%, at least 90%, at least 95%, or up to 100% of neuronal organoids comprise at least the gene expression marker TuJ1. In other embodiments, at least 50% at least 55%, at least 60%, at least 65%, at least 70, at least 75%, at least 80%, at least 85%, at least 90%, at least 95%, or up to 100% of neuronal organoids comprise at least the gene expression marker TuJ1. In still other embodiments, at least 90%, at least 91%, at least 92%, at least 93%, at least 94%, at least 95%, at least 96%, at least 97%, at least 98%, at least 99%, or up to 100% of neuronal organoids comprise at least the gene expression marker TuJ1.

In some embodiments, the neuronal structures comprise at least the gene expression markers class SRY-box 2 (SOX2) and III beta-tubulin (TuJ1). In some embodiments, at least 5%, at least 10%, at least 20%, at least 30%, at least 40%, at least 50%, at least 60%, at least 70%, at least 80%, at least 90%, at least 95%, or up to 100% of neuronal organoids comprise at least the gene expression markers SOX2 and TuJ1. In other embodiments, at least 50% at least 55%, at least 60%, at least 65%, at least 70, at least 75%, at least 80%, at least 85%, at least 90%, at least 95%, or up to 100% of neuronal organoids comprise at least the gene expression markers SOX2 and TuJ1. In still other embodiments, at least 90%, at least 91%, at least 92%, at least 93%, at least 94%, at least 95%, at least 96%, at least 97%, at least 98%, at least 99%, or up to 100% of neuronal organoids comprise at least the gene expression markers SOX2 and TuJ1.

In some embodiments, the genetically modified organoid comprises a kidney organoid. In some embodiments, the kidney organoid comprises at least one nephron structure. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least one nephron structure. In other embodiments, at least 50% at least 55%, at least 60%, at least 65%, at least 70, at least 75%, at least 80%, at least 85%, at least 90%, at least 95%, or up to 100% of kidney organoids comprise at least one nephron structure. In still other embodiments, at least 90%, at least 91%, at least 92%, at least 93%, at least 94%, at least 95%, at least 96%, at least 97%, at least 98%, at least 99%, or up to 100% of kidney organoids comprise at least one nephron structure. Nephron structures can include, but are not limited to, any nephron structures well-known to one of ordinary skill in the art.

In some embodiments, the nephron structures comprise at least distal tubules. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least distal tubules. In other embodiments, at least 50% at least 55%, at least 60%, at least 65%, at least 70, at least 75%, at least 80%, at least 85%, at least 90%, at least 95%, or up to 100% of kidney organoids comprise at least distal tubules. In still other embodiments, at least 90%, at least 91%, at least 92%, at least 93%, at least 94%, at least 95%, at least 96%, at least 97%, at least 98%, at least 99%, or up to 100% of kidney organoids comprise at least distal tubules.

In some embodiments, the nephron structures comprise at least proximal tubules. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least proximal tubules. In other embodiments, at least 50% at least 55%, at least 60%, at least 65%, at least 70, at least 75%, at least 80%, at least 85%, at least 90%, at least 95%, or up to 100% of kidney organoids comprise at least proximal tubules. In still other embodiments, at least 90%, at least 91%, at least 92%, at least 93%, at least 94%, at least 95%, at least 96%, at least 97%, at least 98%, at least 99%, or up to 100% of kidney organoids comprise at least proximal tubules.

In some embodiments, the nephron structures comprise at least podocytes. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least podocytes. In other embodiments, at least 50% at least 55%, at least 60%, at least 65%, at least 70, at least 75%, at least 80%, at least 85%, at least 90%, at least 95%, or up to 100% of kidney organoids comprise at least podocytes. In still other embodiments, at least 90%, at least 91%, at least 92%, at least 93%, at least 94%, at least 95%, at least 96%, at least 97%, at least 98%, at least 99%, or up to 100% of kidney organoids comprise at least podocytes.

In some embodiments, the nephron structures comprise any combination of distal tubules, proximal tubules, or podocytes. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise any combination of distal tubules, proximal tubules, or podocytes. In other embodiments, at least 50% at least 55%, at least 60%, at least 65%, at least 70, at least 75%, at least 80%, at least 85%, at least 90%, at least 95%, or up to 100% of kidney organoids comprise any combination of distal tubules, proximal tubules, or podocytes. In still other embodiments, at least 90%, at least 91%, at least 92%, at least 93%, at least 94%, at least 95%, at least 96%, at least 97%, at least 98%, at least 99%, or up to 100% of kidney organoids comprise any combination of distal tubules, proximal tubules, or podocytes.

In still other embodiments, the nephron structures can be in contiguous arrangement with one or more morphology markers.

In some embodiments, the nephron structures comprise at least distal tubules in contiguous arrangement with at least one distal tubule marker which can include but is not limited to CDH1. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least distal tubules and at least CDH1. In other embodiments, at least 50% at least 55%, at least 60%, at least 65%, at least 70, at least 75%, at least 80%, at least 85%, at least 90%, at least 95%, or up to 100% of kidney organoids comprise at least distal tubules and at least CDH1. In still other embodiments, at least 90%, at least 91%, at least 92%, at least 93%, at least 94%, at least 95%, at least 96%, at least 97%, at least 98%, at least 99%, or up to 100% of kidney organoids comprise at least distal tubules and at least CDH1.

In some embodiments, the nephron structures comprise at least proximal tubules in contiguous arrangement with at least one proximal tubule marker, which can include but is not limited to, Lotus tetragonolobus. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least proximal tubules and at least Lotus tetragonolobus. In other embodiments, at least 50% at least 55%, at least 60%, at least 65%, at least 70, at least 75%, at least 80%, at least 85%, at least 90%, at least 95%, or up to 100% of kidney organoids comprise at least proximal tubules and at least Lotus tetragonolobus. In still other embodiments, at least 90%, at least 91%, at least 92%, at least 93%, at least 94%, at least 95%, at least 96%, at least 97%, at least 98%, at least 99%, or up to 100% of kidney organoids comprise at least proximal tubules and at least Lotus tetragonolobus.

In some embodiments, the nephron structures comprise at least podocytes in contiguous arrangement with at least one podocyte marker, which can include but is not limited to, nephrin. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least podocytes and at least nephrin. In other embodiments, at least 50% at least 55%, at least 60%, at least 65%, at least 70, at least 75%, at least 80%, at least 85%, at least 90%, at least 95%, or up to 100% of kidney organoids comprise at least podocytes and at least nephrin. In still other embodiments, at least 90%, at least 91%, at least 92%, at least 93%, at least 94%, at least 95%, at least 96%, at least 97%, at least 98%, at least 99%, or up to 100% of kidney organoids comprise at least podocytes and at least nephrin.

In some embodiments, the nephron structures comprise at least podocytes in contiguous arrangement with at least one podocyte marker, which can include but is not limited to, podocalyxin. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least podocytes and at least podocalyxin. In other embodiments, at least 50% at least 55%, at least 60%, at least 65%, at least 70, at least 75%, at least 80%, at least 85%, at least 90%, at least 95%, or up to 100% of kidney organoids comprise at least podocytes and at least podocalyxin. In still other embodiments, at least 90%, at least 91%, at least 92%, at least 93%, at least 94%, at least 95%, at least 96%, at least 97%, at least 98%, at least 99%, or up to 100% of kidney organoids comprise at least podocytes and at least podocalyxin.

In some embodiments, the nephron structures comprise at least podocytes in contiguous arrangement with at least one podocyte marker, which can include but is not limited to, nephrin and podocalyxin. In some embodiments, at least 5%, at least 10%, at least 20%, at least 30%, at least 40%, at least 50%, at least 60%, at least 70%, at least 80%, at least 90%, at least 95%, or up to 100% of kidney organoids comprise at least podocytes and at least nephrin and podocalyxin. In other embodiments, at least 50% at least 55%, at least 60%, at least 65%, at least 70, at least 75%, at least 80%, at least 85%, at least 90%, at least 95%, or up to 100% of kidney organoids comprise at least podocytes and at least nephrin and podocalyxin. In still other embodiments, at least 90%, at least 91%, at least 92%, at least 93%, at least 94%, at least 95%, at least 96%, at least 97%, at least 98%, at least 99%, or up to 100% of kidney organoids comprise at least podocytes and at least nephrin and podocalyxin.

In still other embodiments, the modified kidney organoid derived from genetically modified pluripotent stem cells (PSCs) can comprise distal tubule (ECAD+) epithelial cells. In some embodiments, at least 5%, at least 10%, at least 20%, at least 30%, at least 40%, at least 50%, at least 60%, at least 70%, at least 80%, at least 90%, at least 95%, or up to 100% of the epithelial cells in the organoid are distal tubule (ECAD+) epithelial cells. In other embodiments, at least 50% at least 55%, at least 60%, at least 65%, at least 70, at least 75%, at least 80%, at least 85%, at least 90%, at least 95%, or up to 100% of the epithelial cells in the organoid are (ECAD+) distal tubule epithelial cells. In still other embodiments, at least 90%, at least 91%, at least 92%, at least 93%, at least 94%, at least 95%, at least 96%, at least 97%, at least 98%, at least 99%, or up to 100% of the epithelial cells in the organoid are distal tubule (ECAD+) epithelial cells.

In still other embodiments, the modified kidney organoid derived from genetically modified pluripotent stem cells (PSCs) can comprise proximal tubule (LTL+) epithelial cells. In some embodiments, at least 5%, at least 10%, at least 20%, at least 30%, at least 40%, at least 50%, at least 60%, at least 70%, at least 80%, at least 90%, at least 95%, or up to 100% of the epithelial cells in the organoid are proximal tubule (LTL+) epithelial cells. In other embodiments, at least 50% at least 55%, at least 60%, at least 65%, at least 70, at least 75%, at least 80%, at least 85%, at least 90%, at least 95%, or up to 100% of the epithelial cells in the organoid are proximal tubule (LTL+) epithelial cells. In still other embodiments, at least 90%, at least 91%, at least 92%, at least 93%, at least 94%, at least 95%, at least 96%, at least 97%, at least 98%, at least 99%, or up to 100% of the epithelial cells in the organoid are proximal tubule (LTL+) epithelial cells.

In still other embodiments, the modified kidney organoid derived from genetically modified pluripotent stem cells (PSCs) can comprise podocyte (PODXL+) epithelial cells. In some embodiments, at least 5%, at least 10%, at least 20%, at least 30%, at least 40%, at least 50%, at least 60%, at least 70%, at least 80%, at least 90%, at least 95%, or up to 100% of the epithelia cells in the organoid are podocyte (PODXL+) epithelial cells. In other embodiments, at least 50% at least 55%, at least 60%, at least 65%, at least 70, at least 75%, at least 80%, at least 85%, at least 90%, at least 95%, or up to 100% of the epithelia cells in the organoid are podocyte (PODXL+) epithelial cells. In still other embodiments, at least 90%, at least 91%, at least 92%, at least 93%, at least 94%, at least 95%, at least 96%, at least 97%, at least 98%, at least 99%, or up to 100% of the epithelia cells in the organoid are podocyte (PODXL+) epithelial cells.

In still other embodiments, the modified kidney organoid derived from genetically modified pluripotent stem cells (PSCs) can comprise any combination of distal tubule (ECAD+) epithelial cells, proximal tubule (LTL+) epithelial cells, or podocyte (PODXL+) epithelial cells. In some embodiments, at least 5%, at least 10%, at least 20%, at least 30%, at least 40%, at least 50%, at least 60%, at least 70%, at least 80%, at least 90%, at least 95%, or up to 100% of the epithelia cells in the organoid can comprise any combination of distal tubule (ECAD+) epithelial cells, proximal tubule (LTL+) epithelial cells, or podocyte (PODXL+) epithelial cells. In other embodiments, at least 50% at least 55%, at least 60%, at least 65%, at least 70, at least 75%, at least 80%, at least 85%, at least 90%, at least 95%, or up to 100% of the epithelia cells in the organoid can comprise any combination of distal tubule (ECAD+) epithelial cells, proximal tubule (LTL+) epithelial cells, or podocyte (PODXL+) epithelial cells. In still other embodiments, at least 90%, at least 91%, at least 92%, at least 93%, at least 94%, at least 95%, at least 96%, at least 97%, at least 98%, at least 99%, or up to 100% of the epithelia cells in the organoid can comprise any combination of distal tubule (ECAD+) epithelial cells, proximal tubule (LTL+) epithelial cells, or podocyte (PODXL+) epithelial cells.

In still other embodiments, the modified kidney organoid derived from genetically modified pluripotent stem cells (PSCs) can form cysts. In some embodiments, at least 5%, at least 10%, at least 20%, at least 30%, at least 40%, at least 50%, at least 60%, at least 70%, at least 80%, at least 90%, at least 95%, or up to 100% of the organoids can form cysts. In other embodiments, at least 50% at least 55%, at least 60%, at least 65%, at least 70, at least 75%, at least 80%, at least 85%, at least 90%, at least 95%, or up to 100% of the organoids can form cysts. In still other embodiments, at least 90%, at least 91%, at least 92%, at least 93%, at least 94%, at least 95%, at least 96%, at least 97%, at least 98%, at least 99%, or up to 100% of the organoids can form cysts.

In some embodiments, the modified kidney organoid derived from genetically modified pluripotent stem cells (PSCs) can form large cysts. In some embodiments, at least 5%, at least 10%, at least 20%, at least 30%, at least 40%, at least 50%, at least 60%, at least 70%, at least 80%, at least 90%, at least 95%, or up to 100% of the organoids can form large cysts. In other embodiments, at least 50% at least 55%, at least 60%, at least 65%, at least 70, at least 75%, at least 80%, at least 85%, at least 90%, at least 95%, or up to 100% of the organoids can form large cysts. In still other embodiments, at least 90%, at least 91%, at least 92%, at least 93%, at least 94%, at least 95%, at least 96%, at least 97%, at least 98%, at least 99%, or up to 100% of the organoids can form large cysts.

In some embodiments, the large cysts are at least 1 cm in diameter. In still other embodiments, the large cysts are 1 cm in diameter, 1.25 cm in diameter, 1.5 cm in diameter, 1.75 cm in diameter, 2 cm in diameter, 2.25 cm in diameter, 2.5 cm in diameter, 2.75 cm in diameter, 3 cm in diameter, 3.25 cm in diameter, 3.5 cm in diameter, 3.75 cm in diameter, 4 cm in diameter, 4.25 cm in diameter, 4.5 cm in diameter, 4.75 cm in diameter, or greater than 5 cm in diameter.

In still other embodiments, the organoid cyst can comprise any of the markers that are expressed in the organoid as previously described above. In some embodiments, the organoid cyst can express markers that include, but are not limited to, SOX2, TuJ1, CDH1, Lotus tetragonolobus, nephrin, podocalyxin, ECAD+, LTL+, or PODXL+.

In some embodiments, at least 5%, at least 10%, at least 20%, at least 30%, at least 40%, at least 50%, at least 60%, at least 70%, at least 80%, at least 90%, at least 95%, or up to 100% of the organoid cysts can express at least the markers found in the organoid. In other embodiments, at least 50% at least 55%, at least 60%, at least 65%, at least 70, at least 75%, at least 80%, at least 85%, at least 90%, at least 95%, or up to 100% of the organoid cysts can express at least the markers found in the organoid. In still other embodiments, at least 90%, at least 91%, at least 92%, at least 93%, at least 94%, at least 95%, at least 96%, at least 97%, at least 98%, at least 99%, or up to 100% of the organoid cysts can express at least the markers found in the organoid.

### Methods of the Claimed Embodiments

In accordance with the foregoing, in one aspect the disclosure provides a method for making an isolated pluripotent stem cell (PSC) lacking a ciliary structure. In some embodiments, the method can comprise (a) providing the PSC comprising a target gene encoding at least one subunit of kinesin-2; and (b) genetically modifying the PSC to contain a disruption of the target gene encoding at least one subunit of kinesin-2, wherein the disruption produces a genetically modified PSC lacking the ciliary structure.

In some embodiments, the disruption in the target gene encoding at least one subunit of kinesin-2 can comprise contacting the PSC with a targeted nuclease, wherein the nuclease modifies a targeted gene sequence within the target gene.

In some embodiments, the disruption in the target gene encoding at least one subunit of kinesin-2 can comprise contacting the PSC with a nucleic acid sequence encoding a clustered regularly interspersed short palindromic repeat-associated 9 (Cas9) protein and at least two guide ribonucleic acid (gRNA) sequences that hybridize to the targeted gene sequence within the target gene such that the target gene is modified.

In some embodiments, the disruption can comprise introducing a nonsense mutation into the targeted gene sequence.

In some embodiments, the nuclease can comprise a Cas9.

In some embodiments, the target gene is KIF3A and the gRNA targeting KIF3A can comprise a nucleotide sequence as set forth in SEQ ID NO: 1. In some embodiments, the target gene is KIF3B and the gRNA targeting KIF3B can comprise a nucleotide sequence as set forth in SEQ ID NO: 2. In some embodiments, the target gene is KIF3B and the gRNA targeting KIF3B can comprise a nucleotide sequence as set forth in SEQ ID NO: 3. In still other embodiments, the target genes can be KIF3A and KIF3B, and the gRNA targeting KIF3A can comprise a nucleotide sequence as set forth in SEQ ID NO: 1 and the gRNA targeting KIF3B can comprise at least one of the nucleotide as set forth in SEQ ID NO: 2 or SEQ ID NO: 3.

In some embodiments, the disruption of KIF3A, KIF3B, or KIF3A or KIF3B eliminates kinesin-2 protein as detected by immunoblot analysis. In other embodiments, the disruption of KIF3A, KIF3B, or KIF3A or KIF3B eliminate primary cilia as detected by immunofluorescence analysis. In other embodiments, the disruption of KIF3A, KIF3B, or KIF3A or KIF3B eliminates responsiveness to chemical modulation of hedgehog using SAG or cyclopamine and release of ciliary proteins into extracellular vesicles compared to isogenic control cells.

In accordance with the foregoing, in one aspect the disclosure provides a method of differentiating an isolated genetically modified tubular organoid derived from a genetically modified PSC. In some embodiments the method can comprise (a) providing a quantity of genetically modified pluripotent stem cells (PSCs) as described above; (b) culturing the genetically modified PSC in a first culture medium comprising a ROC kinase inhibitor for at least 24 hours and then culturing the PSC sandwiched between two layers of a gelatinous protein mixture secreted by Engelbreth-Holm-Swarm mouse sarcoma cells to form epiblast spheroids, wherein the first medium does not comprise exogenous fibroblast growth factor 2 (FGF2), activin or bone morphogenetic protein; and (c) contacting the epiblast spheroids from step (b) with a second culture medium comprising at least 12 μM CHIR99021 for at least 24 hours, wherein the second medium does not comprise exogenous fibroblast growth factor 2 (FGF2), activin or bone morphogenetic protein, and (d) culturing the epiblast spheroids from step (c) with a third culture medium comprising B27 for at least 48 hours, wherein the third medium does not comprise exogenous fibroblast growth factor 2 (FGF2), activin or bone morphogenetic, thereby differentiating the epiblast spheroids into genetically modified tubular organoids.

In some embodiments, the genetically modified PSCs are cultured in absence of leukemia inhibitory factor (LIF) and doxycycline prior to forming epiblast spheroids. In some embodiments, the genetically modified PSCs are cultured in the presence of Y27632 prior to forming epiblast spheroids. In some embodiments, the genetically modified PSCs in step (b) are cultured in a first culture medium comprising ROC kinase inhibitor for at least 48 hours. In some embodiments, the second culture medium and the third culture medium further comprise RPMI and steps (c) and (d) are performed for a total of at least 7 days. In some embodiments, the culture medium includes MATRIGEL™. In still other embodiments, the culture medium includes collagen I. In some embodiments, culturing the genetically modified PSCs includes depositing a first layer of culture medium on a surface, placing the genetically modified PSCs on the deposited culture medium, and adding a second layer of culture medium over the genetically modified PSCs. In various embodiments, culturing the genetically modified PSCs in a culture medium includes about 1, 2 or more days. In some embodiments, the one or more agents include CHIR99021. In some embodiments, the one or more agents comprise B27. In some embodiments, differentiating the epiblast spheroids includes about 7, 8, 9, 10, 11, 12, or 13 or more days. In some embodiments, the epiblast spheroids express one or more of podocalyxin (PODXL), zonula occluden (ZO-1) and β-catenin. In some embodiments, the epiblast spheroids are cavitated. In some embodiments, the organoids are tubular. In various embodiments, the genetically modified tubular organoids are genetically modified kidney organoids. In some embodiments, the genetically modified tubular organoids lack a ciliary structure. In various embodiments, the organoids express one or more of podocalyxin (PODXL), zonula occluden (ZO-1), and lotus tetragonolobus lectin (LTL).

In some embodiments, genetically modified PSCs can be maintained feeder-free on about 3% Reduced Growth Factor GelTrex™ for at least one passage in media such as mTeSR1, or a hESC conditioned media (CM)+ leukemia inhibitory factor (LIF)+dox for hLR5 iPSCs). In various embodiments, PSCs are primed by withdrawing LIF and doxycycline. In various embodiments, withdrawal of LIF and doxycycline includes substitution with FGF2. In various embodiments, cells are plated a specific density relative to the culture surface and media volume. For example, about 30-60,000 cells/well of a 24-well plate or 4-well chamber slide pre-coated with GelTrex™ in media supplemented with 10 μM Rho-kinase inhibitor Y27632. In another example, about 5-20,000 of a 96-well plate were resuspended in 75 μL of either buffered collagen I (containing 10 mM HEPES and 1×DMEM), reduced growth factor MATRIGEL™ (BD Biosciences), or a 1:1 mixture of the two, incubated for 45 minutes at 37 degrees, and then overlaid with 100 μL of media plus Y27632.

In some embodiments, 48 hours after 3-D culture in the “sandwich” layers of culture medium, PSC epiblast spheroids are differentiated in a differentiation medium including CHIR990021, at a concentration of, for example about 12 μM CHIR, and for a period of about 36 hours. In another example for kidney cell differentiation, the differentiation medium is changed to RB (Advanced RPMI+Glutamax+B27 Supplement) and replaced every three days thereafter. In another embodiment, epiblast spheroids are differentiated in a differentiation medium including CHIR990021, at a concentration of, for example about 12 μM CHIR, and media that is RB minus insulin (RBNI) for a period of about 24 hours, RBNI for 48 hours, addition of 5 μM IWP2 for 48 hours, RBNI for 48 hours, and RB every three days thereafter.

In accordance with the foregoing, in one aspect the disclosure provides a method of testing the effects of compound candidates on a phenotypic organoid model. In some embodiments, the method can comprise generating the phenotypic organoid model on a screening platform comprising the steps of: (a) plating each of a plurality of wells of a culture vessel with a population of genetically modified pluripotent stem cells (PSCs) as described above; (b) differentiating the population of genetically modified PSCs plated in each of the plurality of wells using a single induction step without dissociating or replating the differentiated cells; treating the population of genetically modified PSCs plated in each of the plurality of wells with a therapeutic compound candidate; and testing one or more effects resulting from treatment with each of the therapeutic compound candidates; wherein the method is performed manually using pipettes or automatically by a liquid handling robot.

In some embodiments, the culture vessel can comprise 384 or more wells. In some embodiments the population of genetically modified PSCs are plated at a density of less than 5,000 cells per well. In some embodiments, the single induction step comprises treating the population of genetically modified PSCs in each of the plurality of wells with a concentration of CHIR99021. In some embodiments, the concentration of CHIR99021 is 12 μM. In still other embodiments, the compound candidate can include, but is not limited to, a smoothened agonist or cyclopamine. In some embodiments, the one or more effects resulting from treatment with each of the therapeutic compound candidates include cell toxicity, cell differentiation, or efficacy. In still other embodiments, generating the phenotypic organoid model further comprises treating the population of cells with VEGF.

In certain embodiments, the phenotypic organoid models are generated, optimized, and utilized for testing using an automated system that carries out automated protocols and are compatible with high throughput screening methods. The term “automated” as used herein refers to automation of processes involved in the cell culture including protocols for generating, optimizing, and testing for effects of therapeutic compound candidates. Automation of cell culture protocols is performed fully or partially by liquid handling robots or other instrumentation in order to improve the consistency of the cell culture process and to reduce the chances for cell contamination where there is high volume cell culture needs. Any suitable liquid handling robot or instrumentation such as multi-channel pipettes may be used to execute instructions to carry out the methods and protocols described herein including, but not limited to liquid handling robots, instrumentation and other systems sold by Hamilton Company, Celartia, BioTek, Beckman Coulter, WellMate, CyBio, Integra Biosciences, Agilent Technologies, BMG Labtech, DRG International, Inc., Hudson Robotics, Labcyte, Molecular Devices, Tecan Trading AG, Thermo Fisher Scientific, Bio Molecular Systems, Analytik Jena AG, or any other commercially available system.

Cell culture methods that have traditionally used liquid handling robots are generally shorter in duration than the differentiation process for stem cells, so the use of robots to automate the entire process of plating, differentiation, and other manipulation and/or treatment of human pluripotent stem cells has presented challenges, including programming the robot for long duration experiments and the risk of contamination by fungus or other microbes during the long term handling by the robot. Thus, in one embodiment, the automated methods described herein include a step of introducing an antifungal agent to the cell media during the differentiation process. In certain embodiments, the antifungal agent is Amphotericin B, which may be introduced after the first week of treating the population of cells.

A phenotypic organoid model may be generated for any type of organoid including, but not limited to, kidney organoids, gut organoids, liver organoids, pancreatic organoids, ovary organoids, brain organoids, and cancer organoids. The organoids generated in accordance with the methods described herein may act as a model for a phenotype related to a disease or condition. Each type of organoid may be generated from differentiation of one or more hPSC cell line, and each cell line may require different optimal differentiation conditions to form the phenotypic organoid. Thus, automated methods for optimizing differentiation cell line are provided herein to optimize a desired cell line for use in a high throughput screening system.

In some embodiments, generating the phenotypic organoid model includes a step of plating one or more wells of a high-throughput culture vessel with a low-density population of human pluripotent stem cells (hPSCs). The population of hPSCs may be a population from any suitable hPSCs cell line including, but not limited to, a primary human embryonic stem cell line (hESCs, e.g. the H9 ES cell line), an induced pluripotent stem cell line (iPSC, e.g. the WTC1 1 iPS cell line), or a genetically modified hPSC cell line. The high-throughput culture vessel may be of any size suitable for high-throughput screening or testing and may include a microwell cell culture plate having 96 wells, 384 wells, 1536 wells, 3456 wells, 9600 wells, or any other large format microwell culture plate. In certain embodiments, the high-throughput culture vessel is a 384 well microwell culture plate or a plate that includes more than 384 wells.

The plates can first be coated with Matrigel, diluted 1:100 in cold DMEM/F12, and then added to each well of the high-throughput culture vessel at a volume of 30 pL per well. The dilution of Matrigel used in the embodiments described herein was modified from the typical dilution to reduce cell clumping effects of other dilutions such as a 1:60 dilution.

After initially plating the cells, the method includes a differentiating step, whereby the population of hPSCs are differentiated using a differentiating factor specific to the desired somatic cell types that make up the desired organoid. For example, in one embodiment, the desired organoid is a kidney organoid and the differentiating factor is a CHIR factor such as CHIR 99021. In the automated methods described herein, the CHIR treatment is generally shorter than typical differentiation methods and is added at a higher volume and concentration than is typical for lower-throughput plates. For example, treatment with a CHIR compound may be at 14 mM and up to about 6 hours shorter than normal treatment (˜20% of the total treatment time). Further, the differentiating step can be a single induction step without dissociating or replating the differentiated cells as discussed below.

In some embodiments, the method can include adding additional phenotypic factors to stimulate a phenotypic change in the organoid development. For example, VEGF can be added in order to enhance endothelial cell differentiation in an organoid model as discussed in the examples below. Other microenvironmental factors such as 8-bromoadenosine, cyclic adenosine monophosphate (cAMP), forskolin, or blebbistatin, which induces cysts in kidney organoids, may be introduced to the population of cells as well including, but not limited to the factors discussed below in the working examples.

In certain embodiments, the organoid models generated by the methods and protocols described herein model a disease or condition that causes cysts to form on or in an affected tissue or organ (i.e. a cystogenic disease or condition). In some embodiments, conditions or diseases that may cause cysts to form may include, but are not limited to, genetic conditions, tumors, infections, errors in embryonic development, cellular defects, chronic inflammatory conditions, blockages of ducts in the body, parasites, and injuries to skin or vessels. According to some embodiments, certain types of cysts that are caused by the disease or condition may form the basis of the phenotypic organoid model and include, but are not limited to, acne cysts, arachnoid cysts, Baker's cysts, Bartholin's cysts, breast cysts, Chalazion cysts, colloid cysts, dentigerous cysts, dermoid cysts, epidiymal cysts, ganglion cysts, hydatid cysts, ovarian cysts, pancreatic cysts, periapical cysts, pilar cysts, pilonidal cysts, renal (or kidney) cysts, autosomal dominant PKD, autosomal recessive PKD, ciliopathy syndromes, Bardet Biedl Syndrome, Joubert Syndrome, nephronophthisis, polycystic liver disease, pineal gland cysts, sebaceous cysts, tarlov cysts (also known as perineural or perinurial cysts), vocal fold cysts (e.g., mucus retention cysts, epidermoid cysts).

In some embodiments, each of a plurality of wells in the vessel can be treated with a therapeutic compound candidate, then evaluated for one or more effects of that treatment. Among other things, the method allows for testing of cell toxicity of the compound, its effect on the phenotype of the organoid, and/or the efficacy of the compound. In certain embodiments, each well may be treated with a different compound and the same effect may be tested for each compound. Alternatively, certain wells on a single culture vessel may be treated with the same compound, and different effects of the compound may be tested on the same culture vessel.

In accordance with the foregoing, in one aspect the disclosure provides a method for measuring organ specific toxicity and disease phenotypes of an agent on a genetically modified organoid, the method comprising: (a) providing one or more genetically modified organoids derived from a genetically modified pluripotent stem cell (PSC) in a high throughput format; (b) admixing the agent with the one or more genetically modified organoids; and (c) detecting one or more outcomes of agent on the one or more genetically modified organoids wherein the one or more outcomes indicates toxicity, disease, differentiation state, or a combination thereof of the one or more genetically modified organoids.

In some embodiments, the method comprises admixing one or more additional agents with the one or more genetically modified organoids and detecting one or more additional outcomes on the one or more genetically modified organoids. In some embodiments, an outcome is differentiation state of the one or more genetically modified organoids. In other embodiments, the providing of one or more genetically modified organoids is an adherent culture format. In still other embodiments, the one or more genetically modified organoids are derived from human iPSCs. In some embodiments, the one or more organoids are genetically modified kidney organoids. In some embodiments, the method can comprise performing single-cell RNA-seq on the one or more genetically modified organoids. In still other embodiments, the one or more outcomes comprises phenotypic screening of the one or more genetically modified organoids.

In accordance with the foregoing, in one aspect the disclosure provides a method of screening a compound for an effect on tubular organoids, including providing a quantity of tubular organoids, adding one or more compounds to the tubular organoids, determining changes to phenotype or activity of the tubular organoids, and correlating the changes with an effect of the compounds on tubular organoids, thereby screening the one or more compounds for an effect on tubular organoids.

In various embodiments, determining changes to phenotype or activity includes detecting one or more markers in the tubular organoids. In various embodiments, the one or more markers comprise kidney injury molecule (KIM-1). In various embodiments, an increase in KIM-1 expression correlates with a toxic effect of the compound. In various embodiments, the tubular organoids are kidney organoids.

In accordance with the foregoing, in one aspect the disclosure provides a method of identifying a disrupted signaling pathway contributing to polycystic kidney disease (PKD), the method comprising: (a) differentiating genetically modified kidney organoids to day 18; (b) performing transcriptome analysis and/or proteomic analysis on the partitioned 18 day old genetically modified kidney organoids; (c) using transcriptome analysis and/or proteomic analysis to identify at least one differentially expressed gene; (d) comparing results from the transcriptome analysis and/or the proteomic analysis from step (c) to transcriptome analysis and/or proteomic analysis results of (i) a non-genetically modified kidney organoid; (ii) a polycystic kidney disease knockout; and (iii) a non-genetically modified kidney organoid and a polycystic kidney disease (PKD) knockout; identifying a signaling pathway that is expressed in (i) the genetically modified kidney organoid and (ii) determining whether the disrupted signaling pathway is similar to the disrupted pathway in the PKD knockout, thereby identifying a singling pathways contributing to PKD.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); Ran, F. A., et al., Genome engineering using the CRISPR-Cas9 system, Nature Protocols, 8:2281-2308 (2013), and Jiang, F. and Doudna, J. A., CRISPR-Cas9 Structures and Mechanisms, Annual Review of Biophysics, 46:505-529 (2017) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following example is set forth so as to provide those of ordinary skill in the art with a complete disclosure and description of how to make and use the present invention and are not intended to limit the scope of what the inventors regard as their invention nor are they intended to represent that the experiments below are all or the only experiments performed.

### Example 1

This Example describes producing and characterizing genetically modified human pluripotent stem cells lacking cilia and using these cells to generate genetically modified organoids, as well as characterizing these genetically modified organoids.

Generation of Stable hPSCs without Cilia

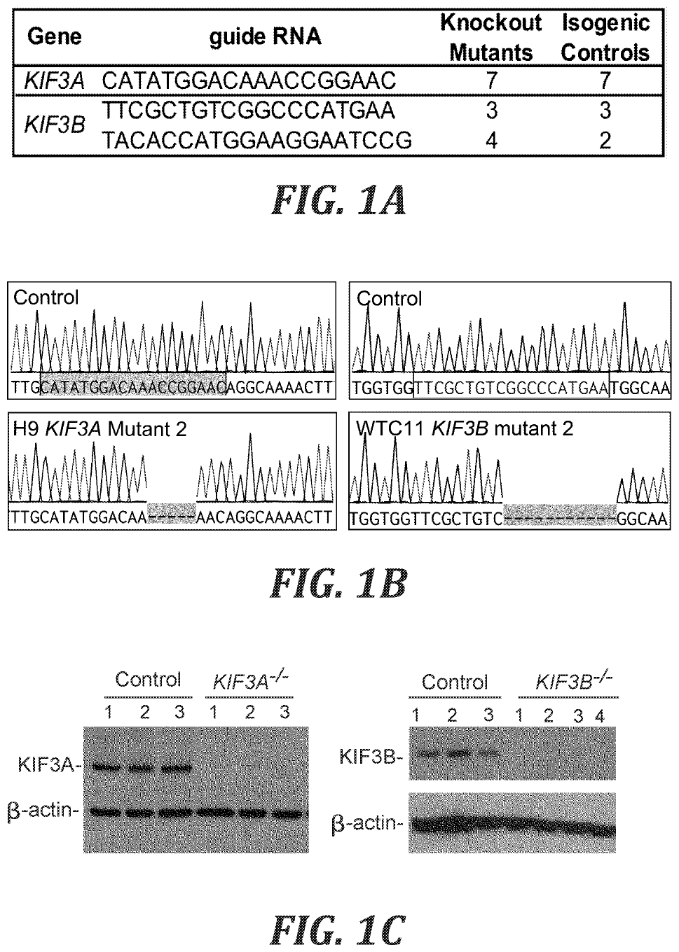

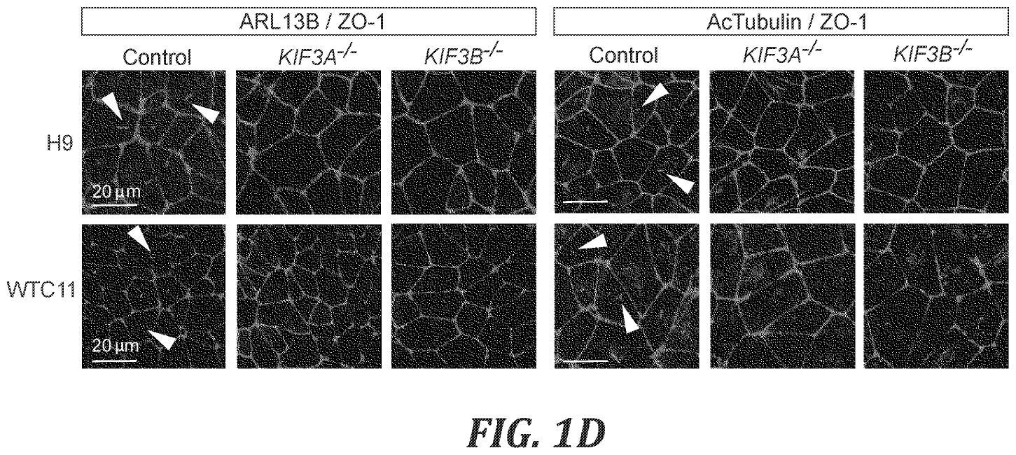

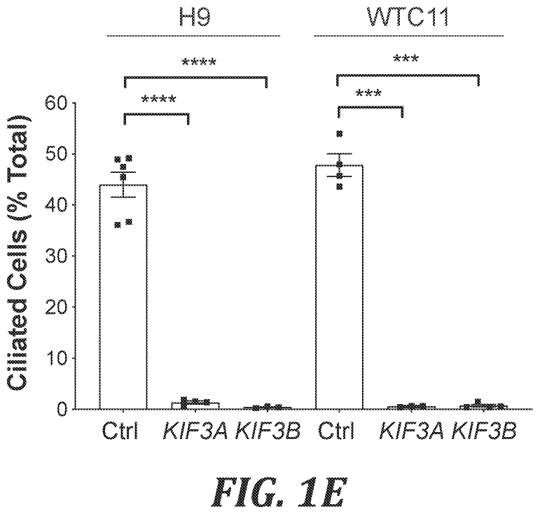

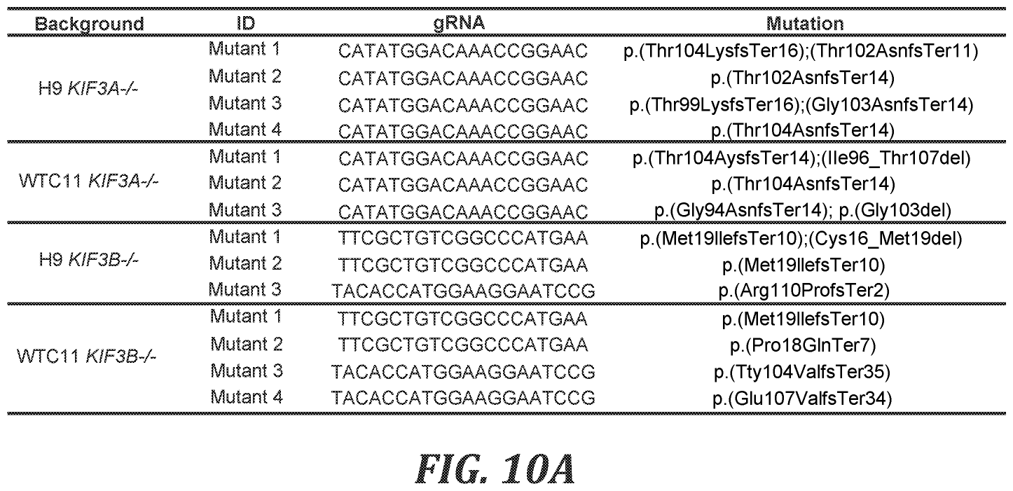

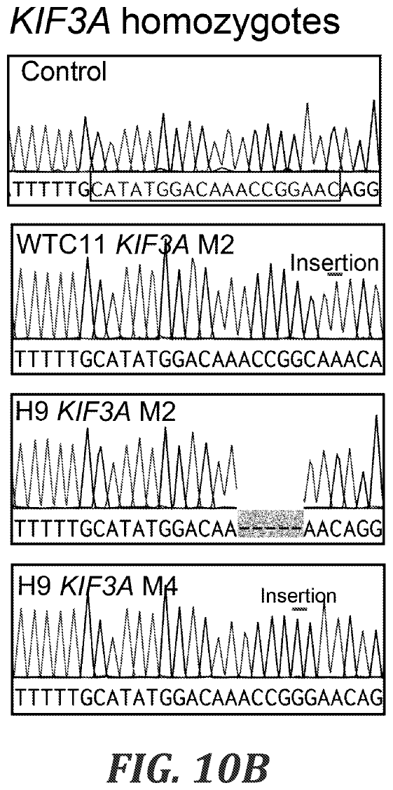

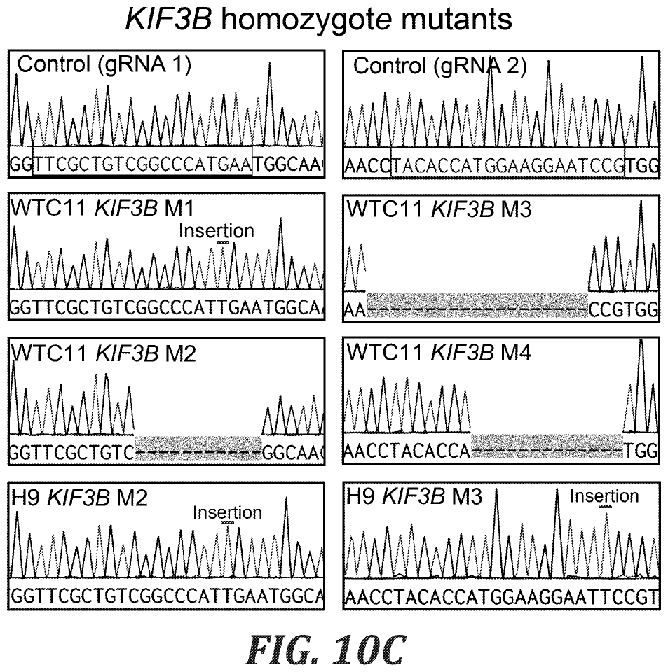

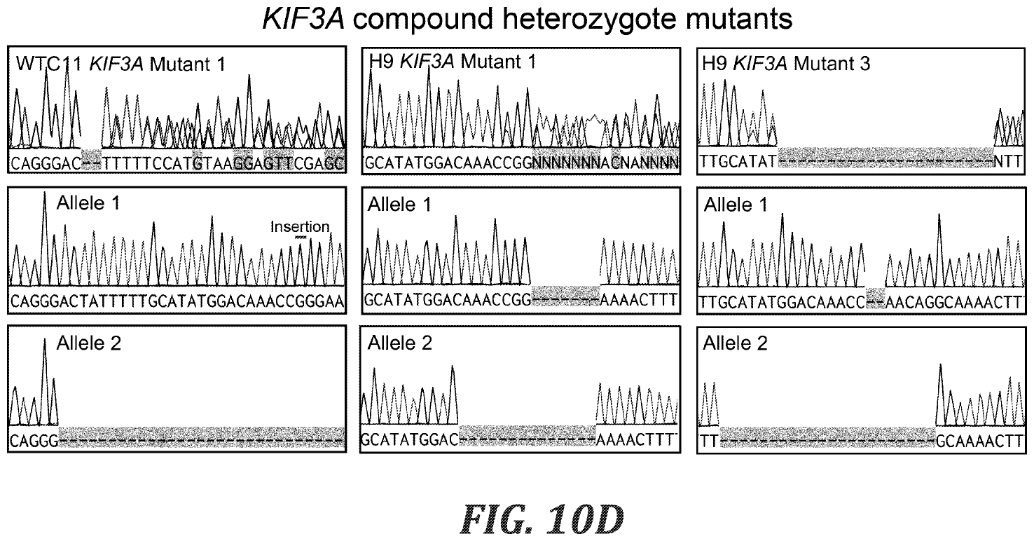

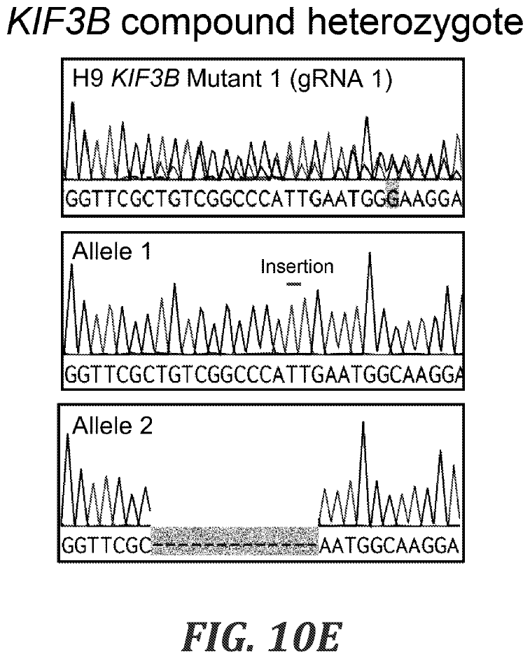

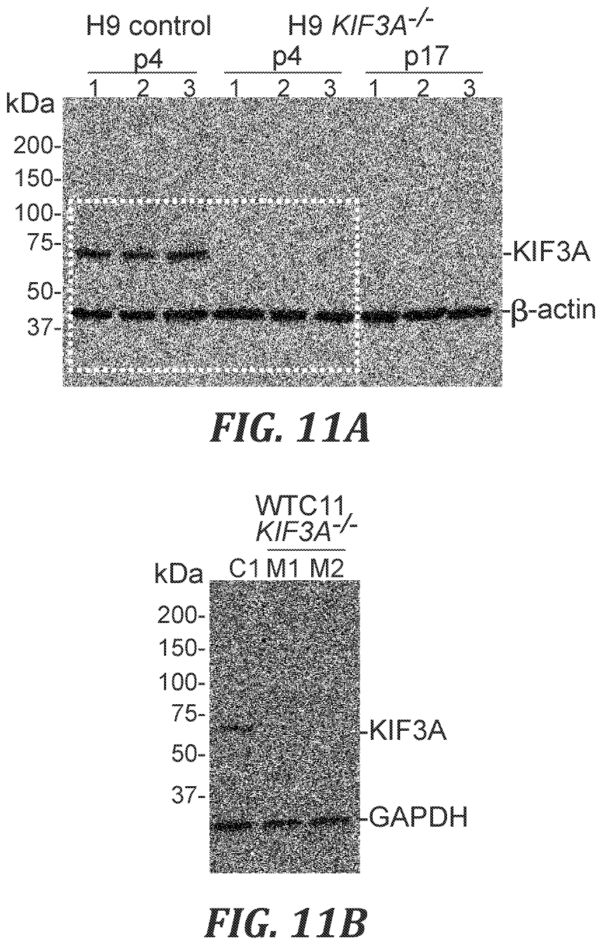

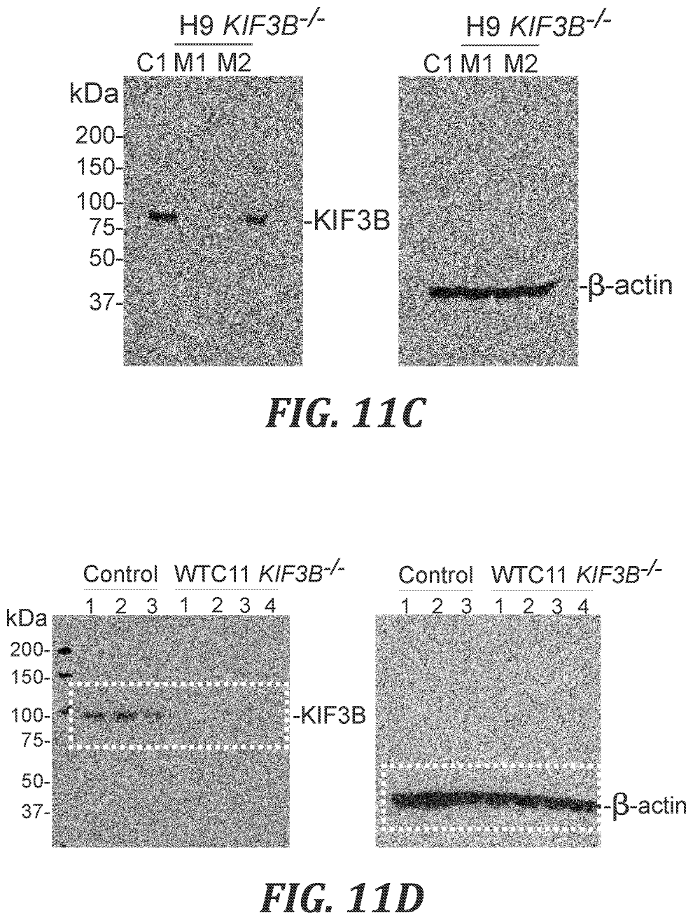

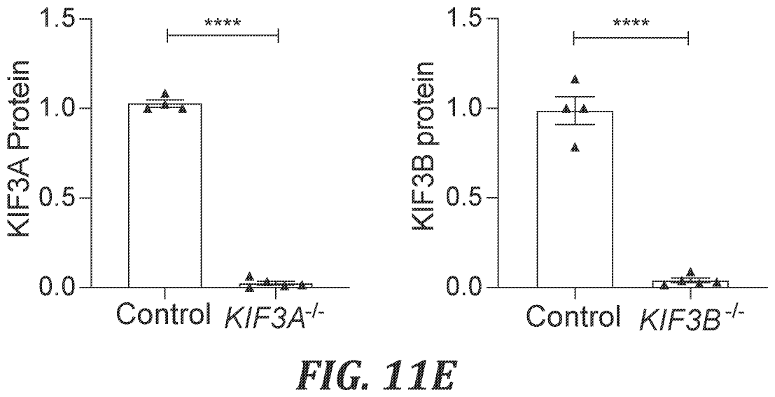

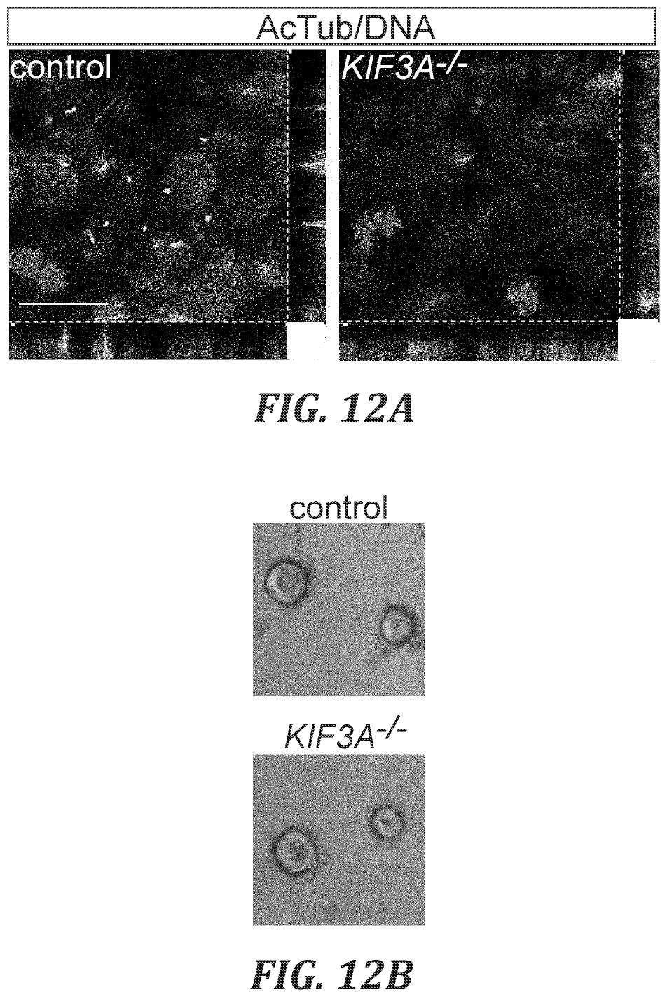

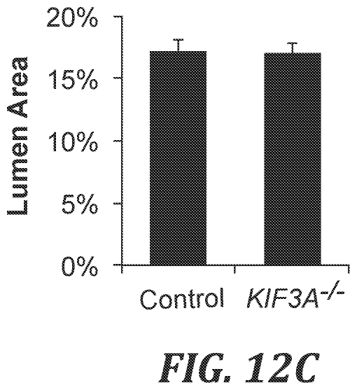

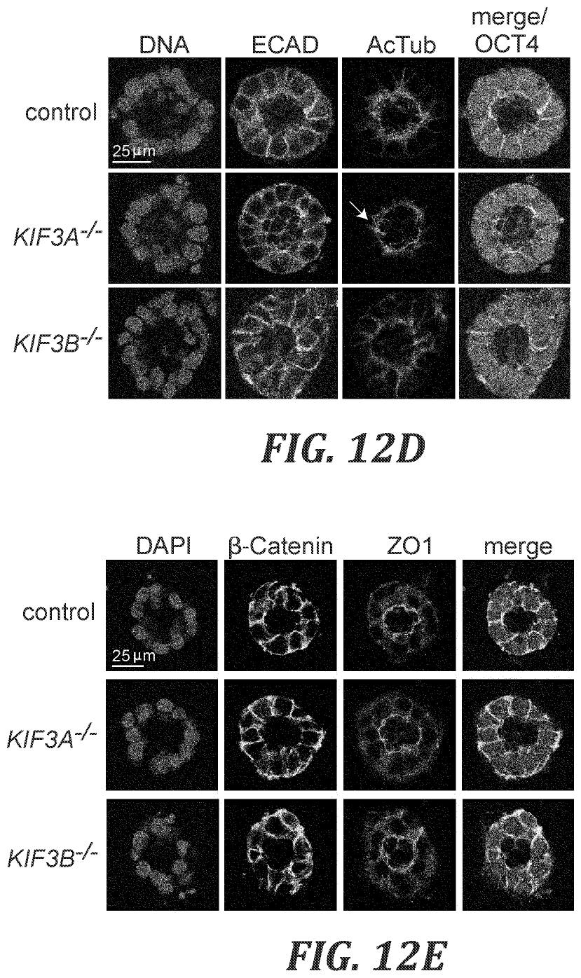

To establish a general tool for studying the function of human cilia, a genetic strategy was used to completely ablate these organelles in hPSCs. By applying the CRISPR-Cas9 gene-editing system to hPSCs, nonsense mutations were introduced in the KIF3A or KIF3B genes. Three distinct guide RNAs were applied to male or female hPSCs to generate a collection of fourteen different mutant cell lines, plus twelve isogenic control cell lines of matching passage and experimental history (FIGS. 1A and 10A). Each cell line was subjected to allele-specific sequencing to determine every knockout mutation (FIG. 1B, 10B-E). KIF3A and KIF3B proteins were expressed in unmodified controls, but the targeted protein was undetectable in each of the gene-edited hPSC lines (FIG. 1C, FIG. 11A-E). Whereas ˜ 50% of control hPSCs formed ciliary axonemes (ARL13B⁺ and acetylated α-tubulin⁺), cilia were completely undetectable in the gene-edited hPSCs of both targeted genes in both genetic backgrounds (FIG. 1D-E, FIG. 12A). This collection of mutants is referred to as kinesin-2 knockout hPSCs.

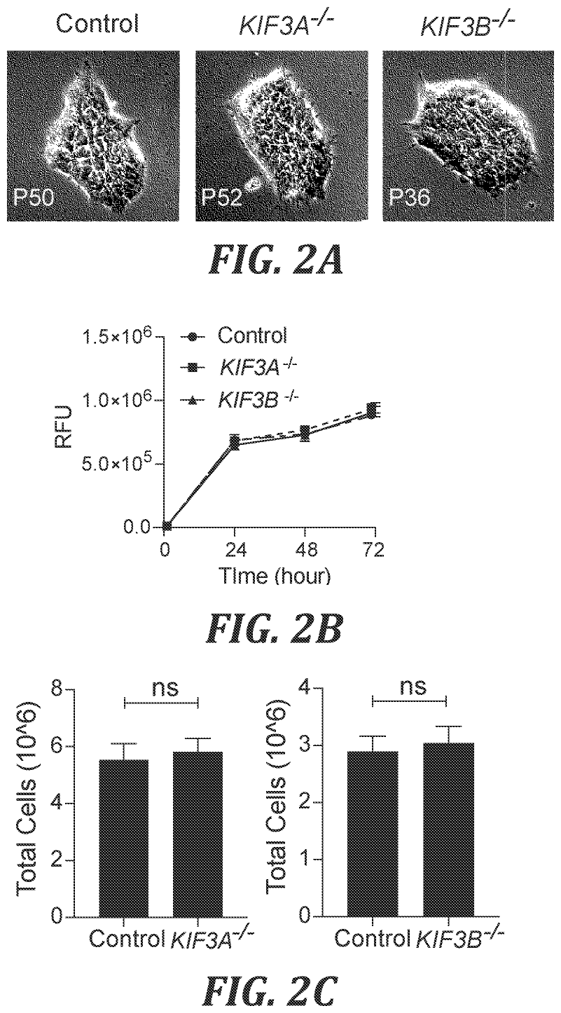

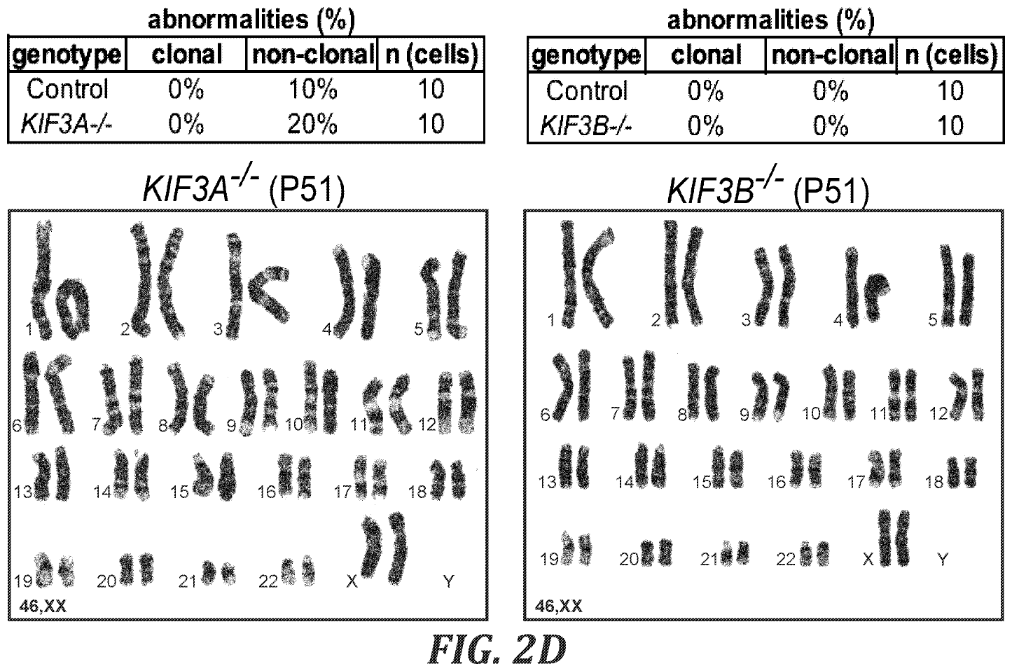

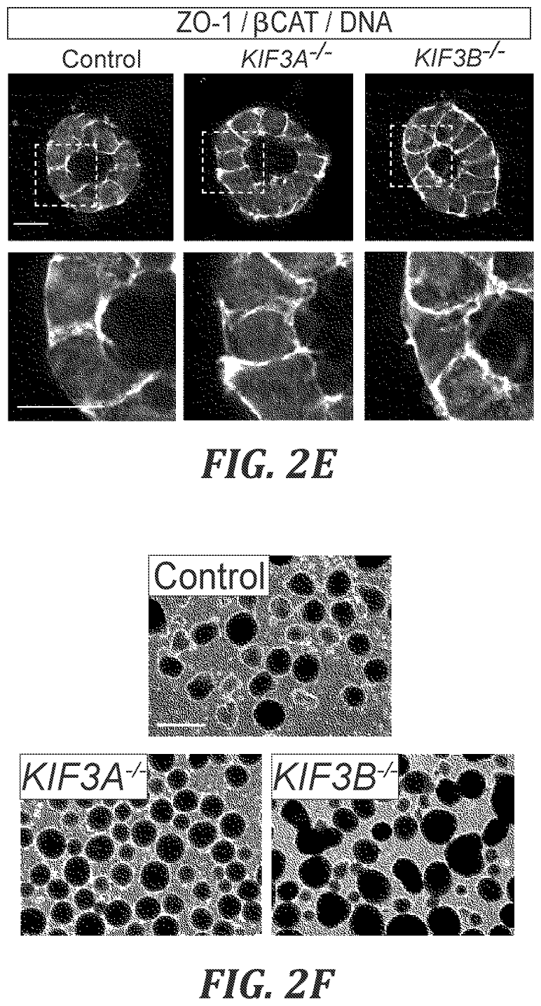

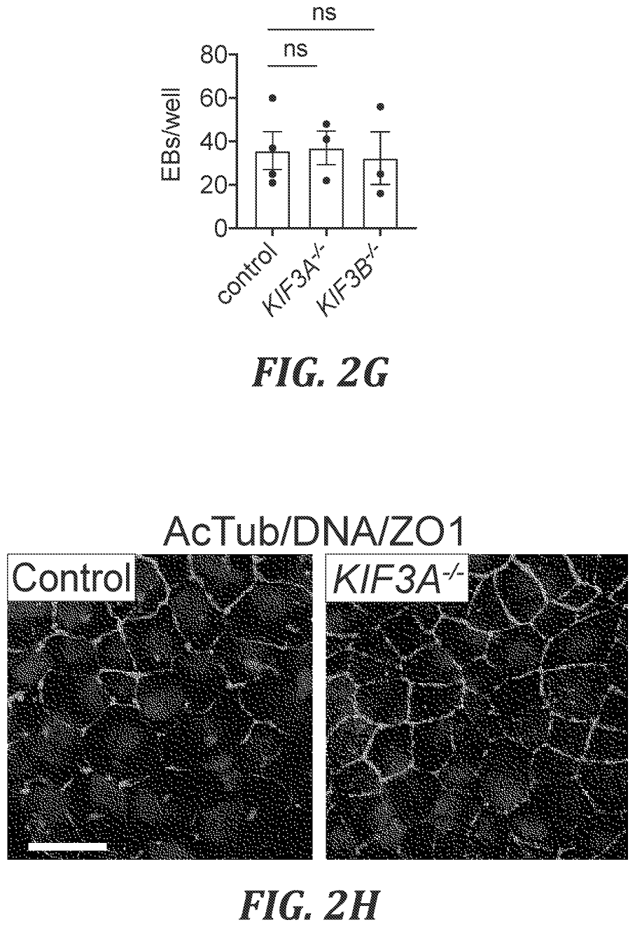

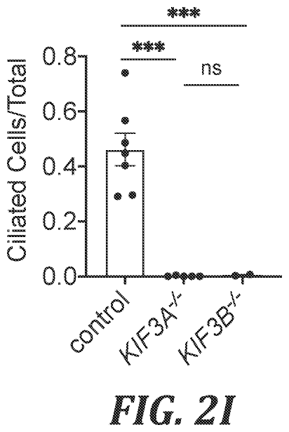

Both KIF3A^{-/-} and KIF3B^{-/-} hPSCs formed flat, smooth-edged colonies with characteristic hPSC morphology, which remained stable and self-renewing with no signs of aberrant differentiation or senescence in over fifty passages in vitro (FIG. 2A). Growth rates were indistinguishable from isogenic controls (p=0.69), and karyotyping detected no significant increase in chromosomal abnormalities (FIG. 2B-D). When cultured in three-dimensional matrix, kinesin-2 knockout hPSCs formed pluripotent epiblast spheroids with hollow, amniotic cavities of normal size and polarity, despite the loss of cilia from the luminal surface (FIG. 2E, FIG. 12B-E). Thus, cilia were dispensable in undifferentiated hPSCs.

Kinesin-2 Knockout hPSCs Produce Diverse Cell Types Lacking Cilia

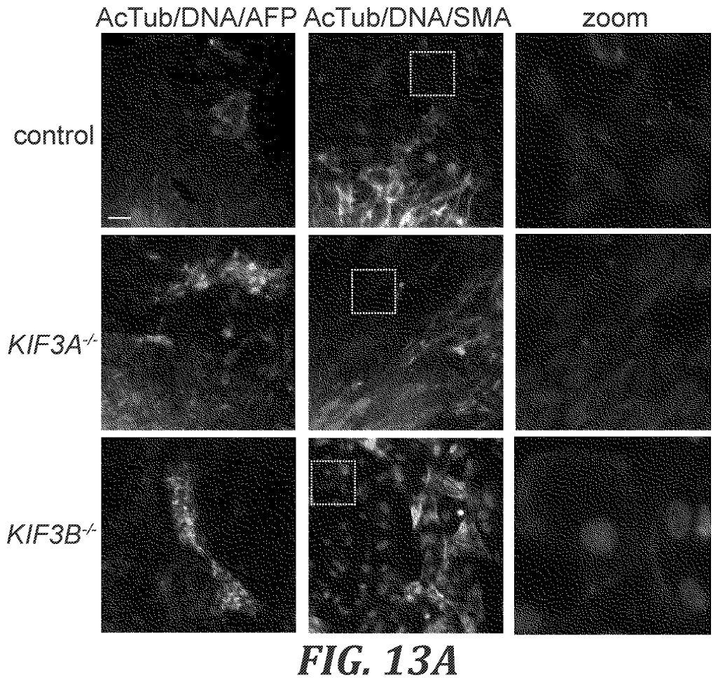

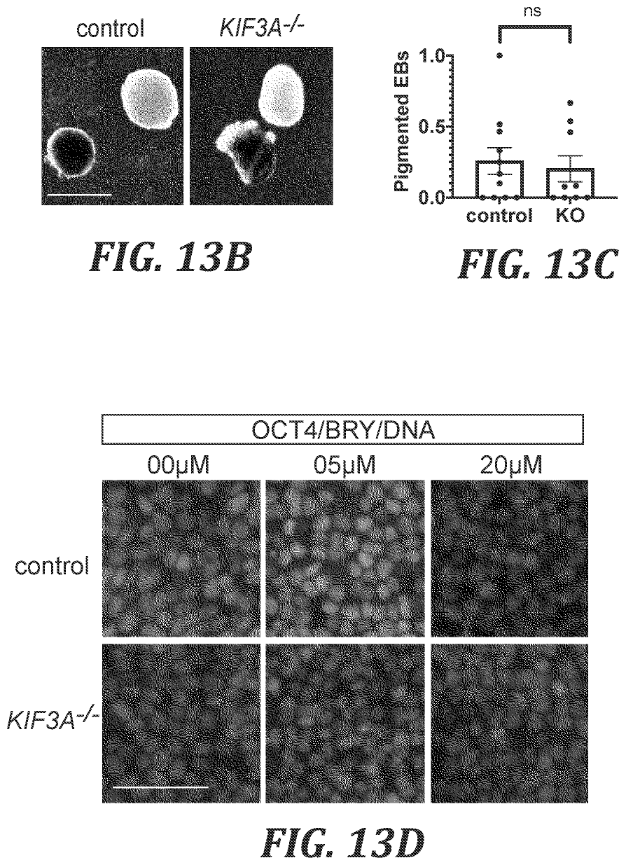

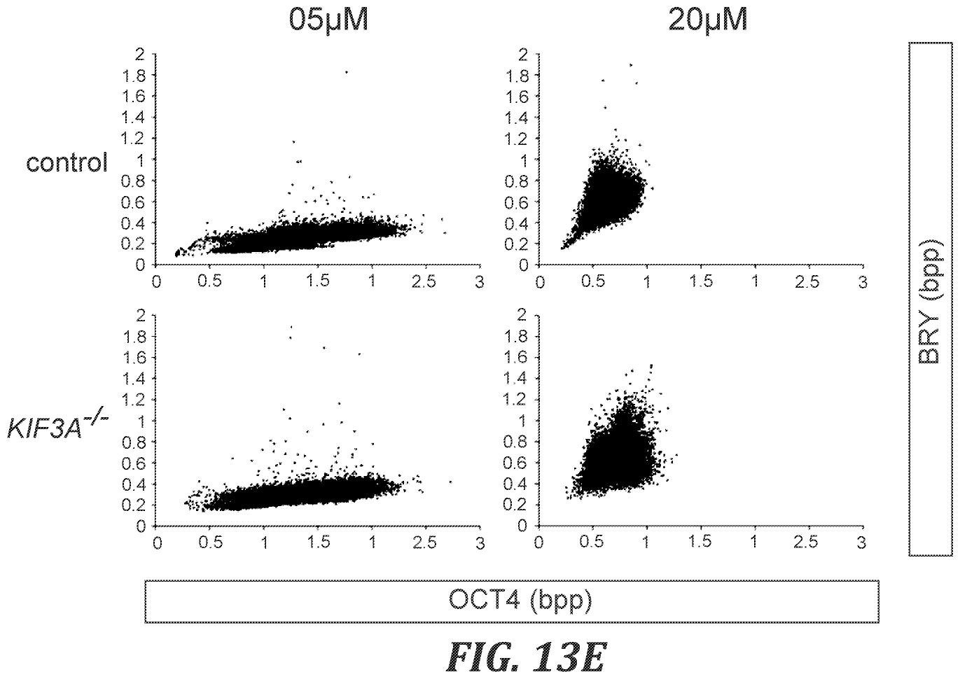

When switched to differentiating culture conditions, both kinesin-2 knockout hPSCs and controls differentiated efficiently into embryoid bodies (EBs) containing cores of ectoderm (neuroepithelium) bordering migratory outgrowths of endoderm (β-fetoprotein, AFP) and mesoderm (smooth muscle α-actin, SMA) (FIG. 2F-G, FIG. 13A). Primary cilia were detected in control EBs, but not in KIF3A^{-/-} or KIF3B^{-/-} EBs, indicating that these lineages formed without cilia (FIG. 2H-I, FIG. 13A). In prolonged cultures, both control and kinesin-2 knockout EBs formed patches of pigmented epithelium (FIG. 13B). No significant difference was observed in the ability of kinesin-2 knockout hPSCs to form EBs (p=0.92) and pigmented cells (p=0.68), nor in their dose-dependent upregulation of brachyury (BRY) in response to CHIR99021 (FIG. 2G, FIG. 13C-E). These mutant hPSCs were therefore pluripotent and provided a stable and renewable source of diverse cell types.

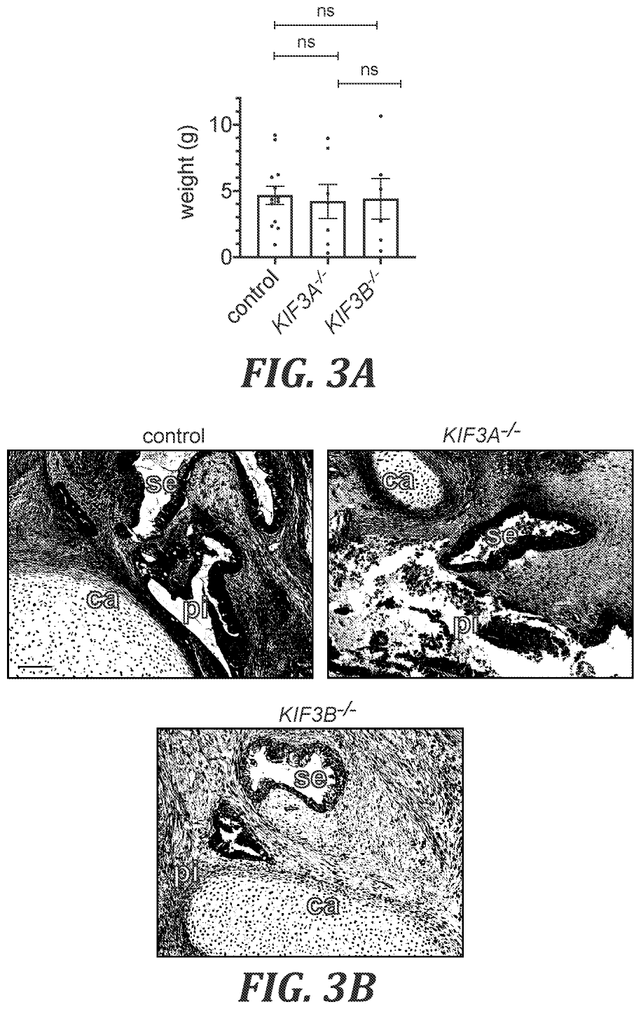

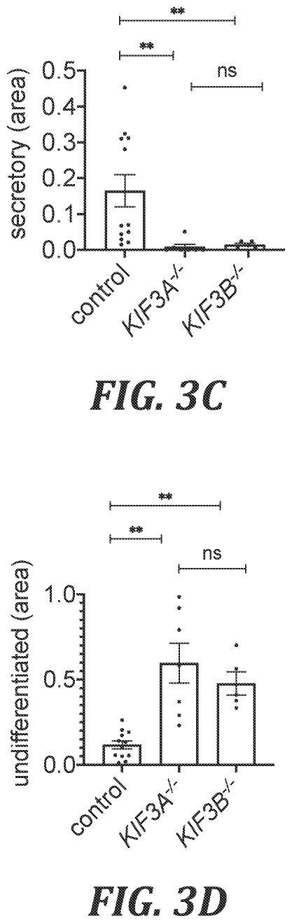

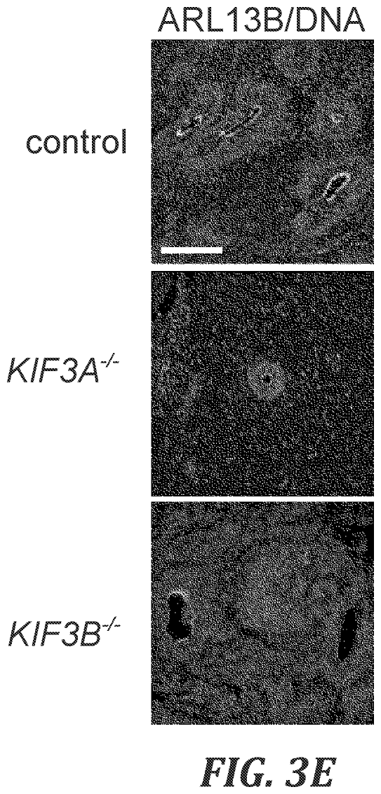

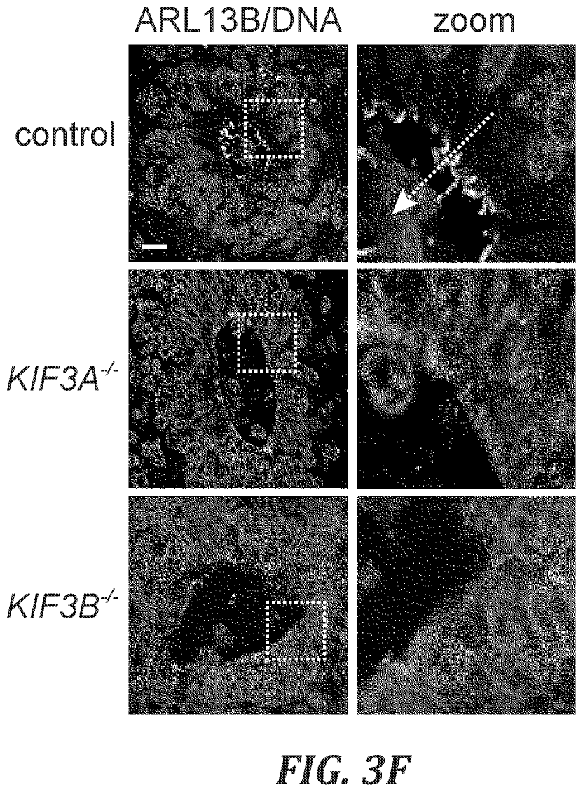

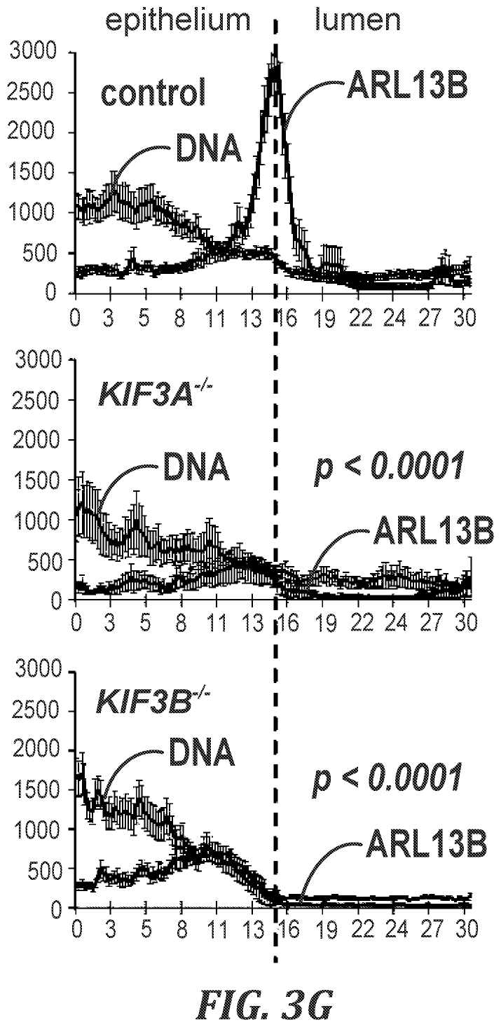

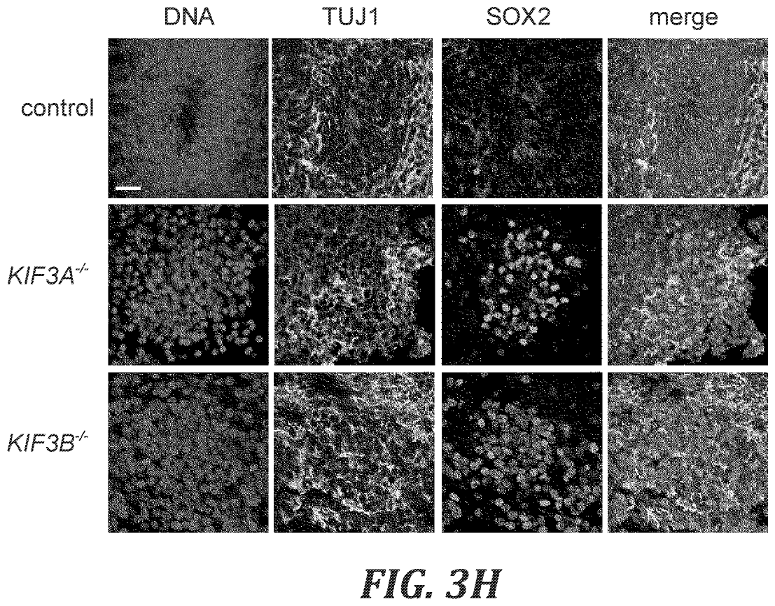

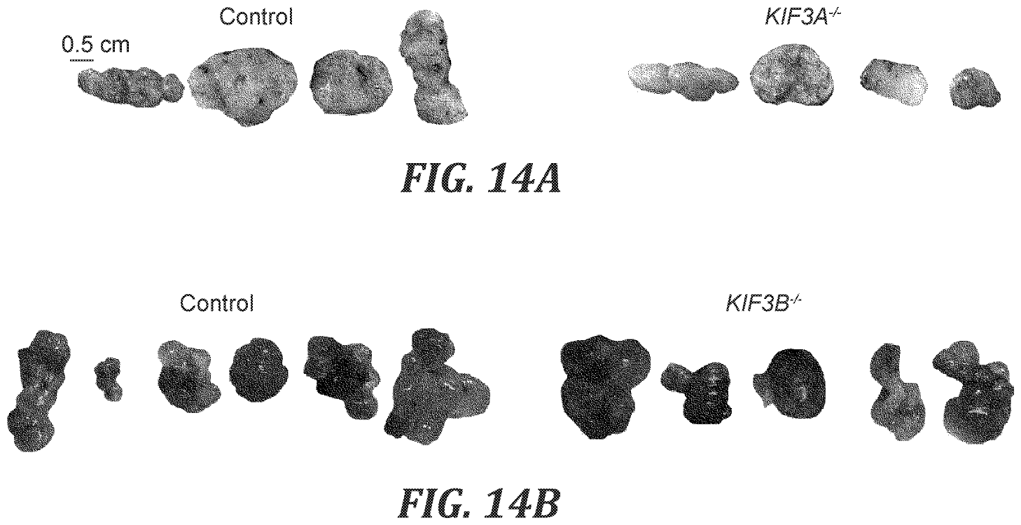

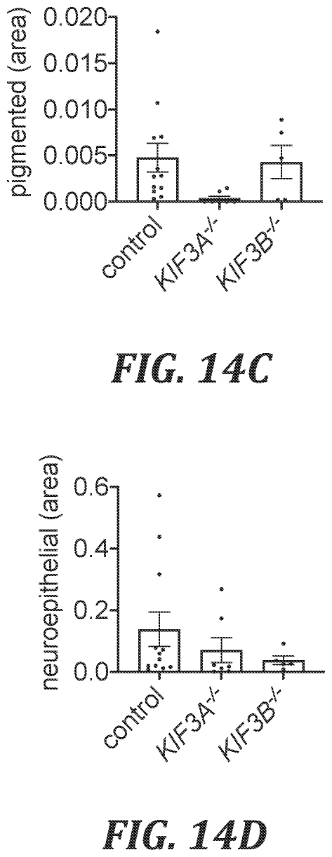

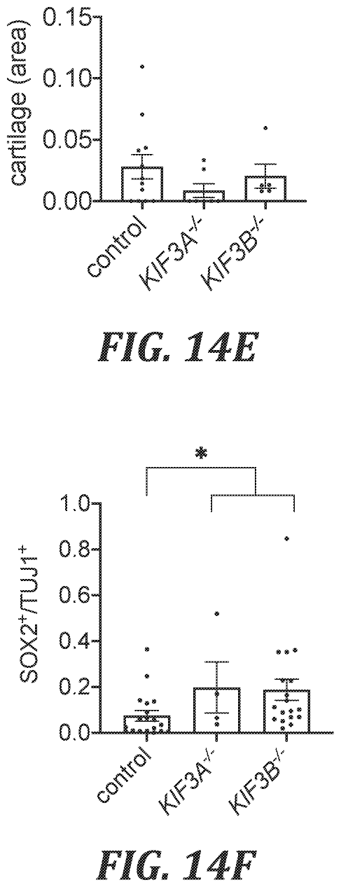

EB culture involves complex media containing high concentrations of proteins and growth factors. To provide a more physiological environment for differentiation, hPSCs were implanted into immunodeficient animals in vivo. Both kinesin-2 knockout hPSCs and isogenic controls rapidly formed large tumors under these conditions (FIG. 14A-B). Growths from kinesin-2 knockout hPSCs were of similar size and weight to controls (p=0.75) and contained a diversity of complex and well-differentiated tissues typical of teratomas, including pigmented epithelia and cartilage (FIG. 3A-B). However, secretory epithelia were reduced >10-fold in growths derived from kinesin-2 knockout hPSCs, while undifferentiated blasts, which stained primarily with hematoxylin rather than eosin, were ˜ 5-fold more prevalent (FIG. 3C-D). These differences were statistically significant (p<0.01). Quantities of pigmented epithelium, cartilage, and neuroepithelium derived from kinesin-2 knockout hPSCs were less than or equal to controls (FIG. 14C-E).

We further examined ADP-ribosylation factor-like GTPase 13b (ARL13B), which is associated with Joubert syndrome in humans and ventricular organization defects in animals. In controls, ARL13B localized brightly to multi-ciliated arrays at the lumens of neuroepithelial rosettes, whereas in rosettes derived from KIF3A^{-/-} or KIF3B^{-/-} hPSCs, multi-cilia were absent, and ARL13B was restricted to a dim halo around the lumen, where its intensity was significantly reduced (FIG. 3E-G). In patches of TUJ1⁺ neurons, increased quantities of SOX2⁺ neuronal stem cells were detected in growths from kinesin-2 knockout hPSCs, compared to isogenic controls (FIG. 3H, FIG. 14F). These experiments revealed a critical role for cilia in tissue differentiation in vivo.

### Kidney Organoid Differentiation Depends Upon Kinesin-2

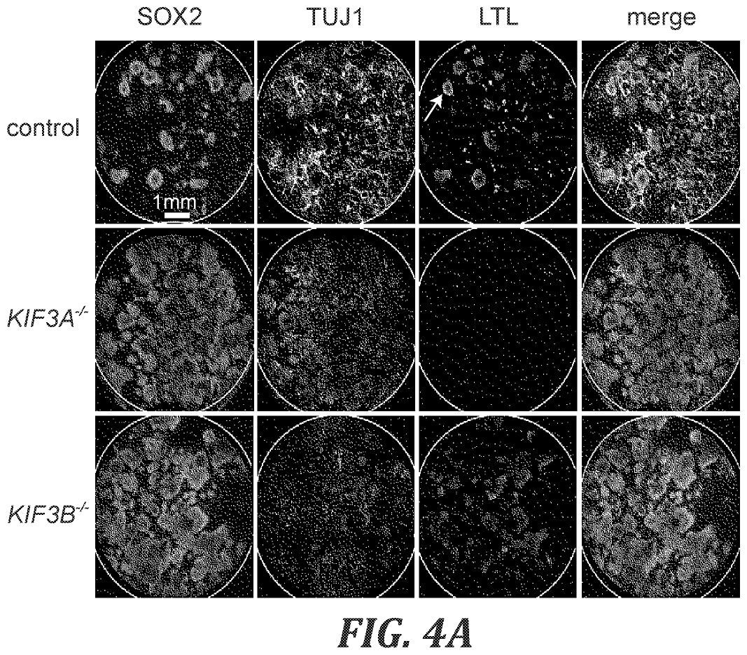

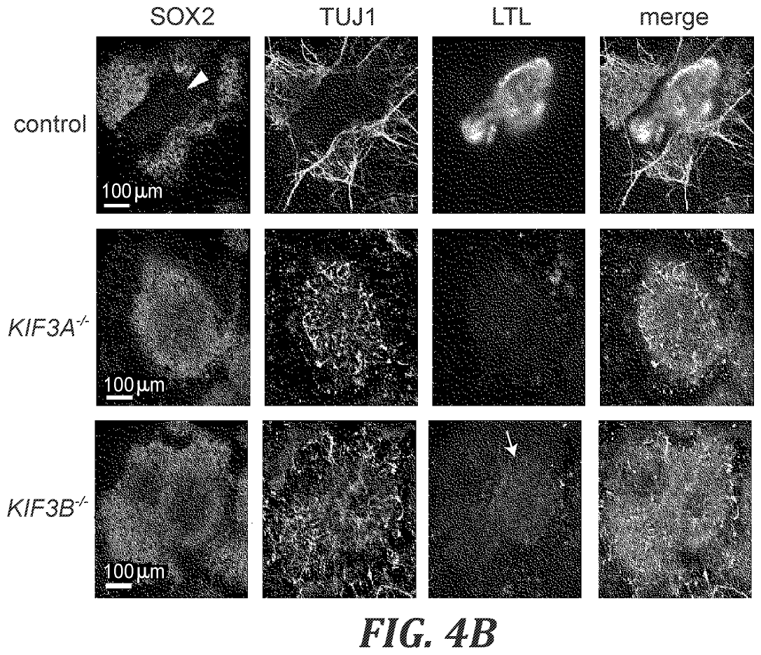

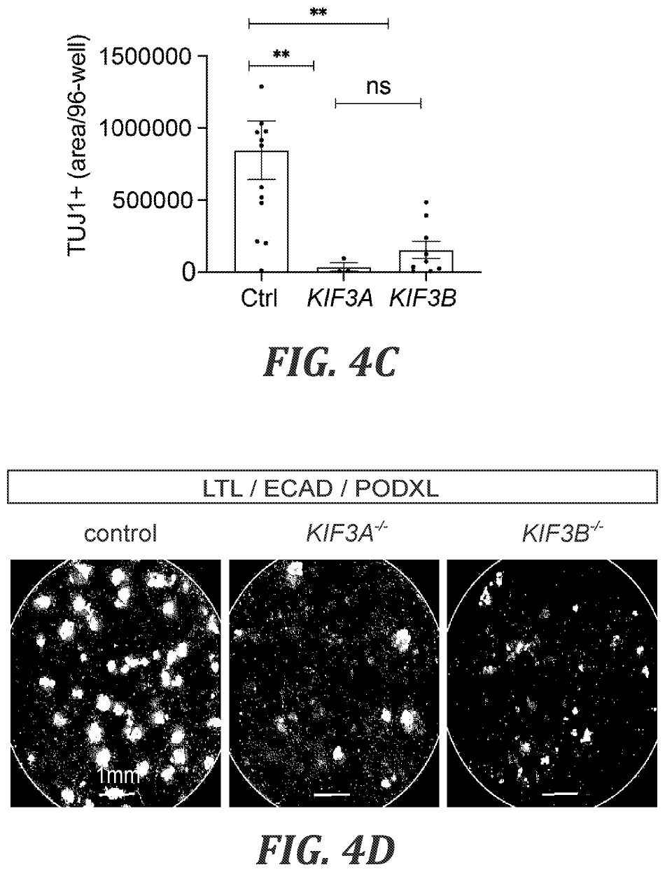

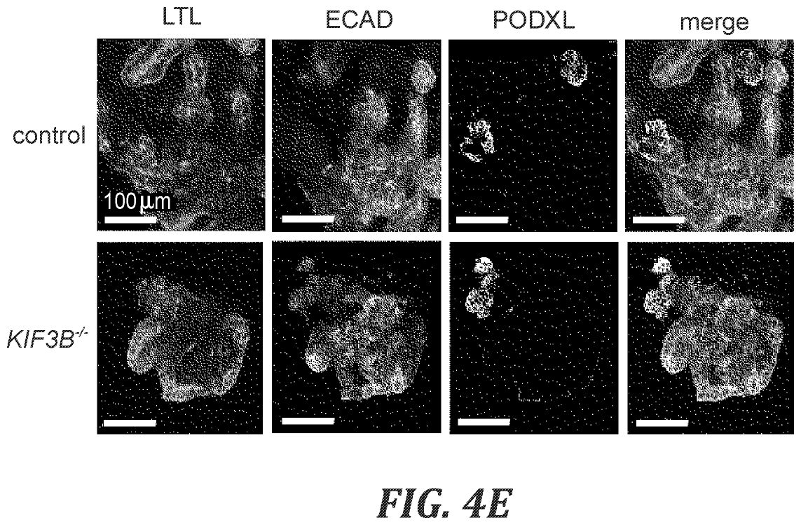

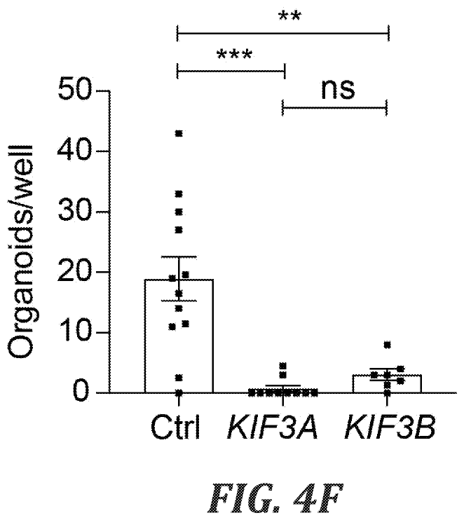

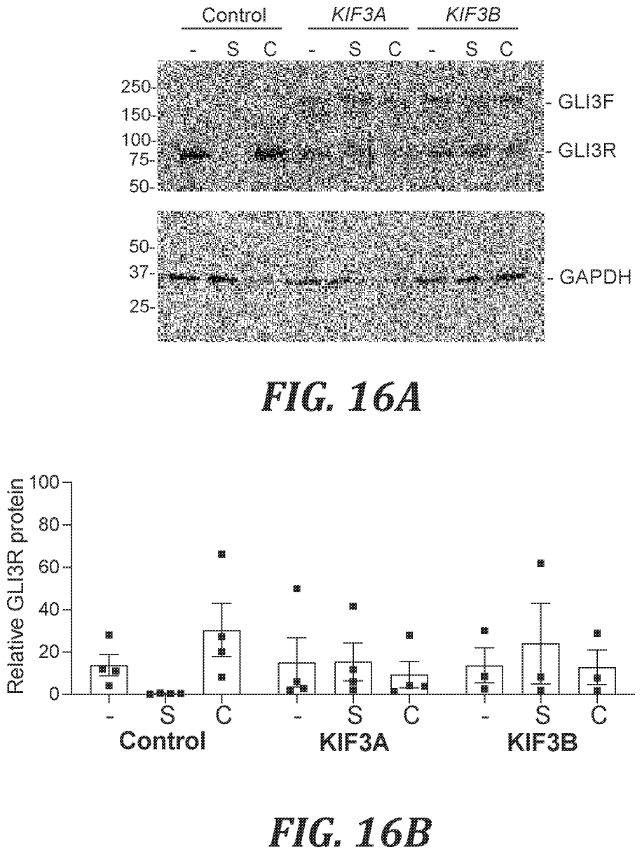

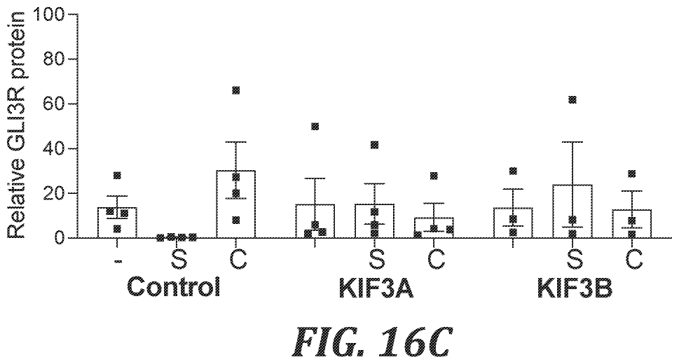

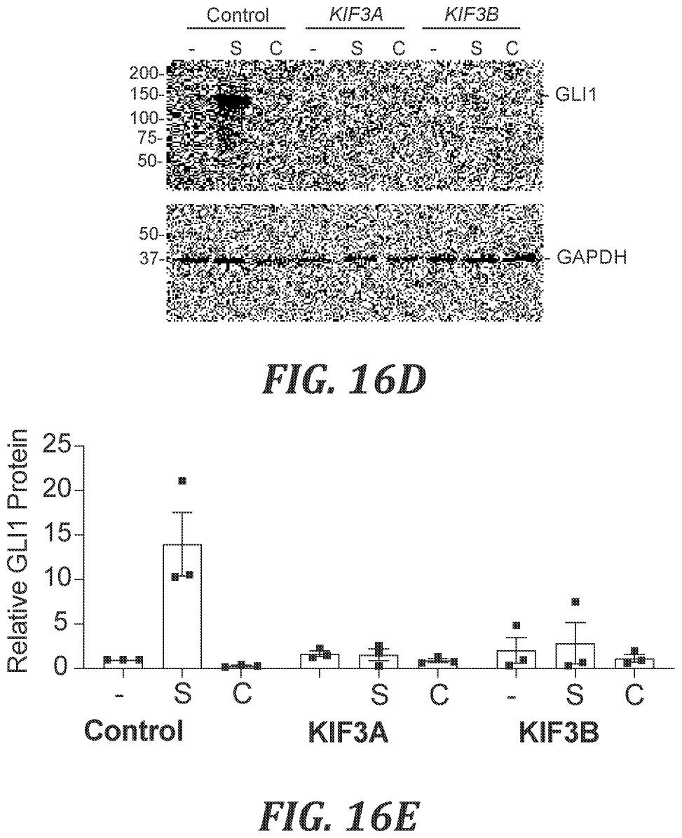

Teratomas are naturally heterogenous and difficult to perturb experimentally. To elicit tissue-like phenotypes in vitro, a kidney organoid differentiation protocol with defined, minimal media components and no FGF was applied to hPSCs, which produces neuronal clusters alongside nephron-like organoids. Compared to isogenic controls, both KIF3A^{-/-} and KIF3B^{-/-} cultures showed a significant decrease in TUJ1⁺ neurons, despite the presence of abundant SOX2⁺ progenitors (FIG. 4A-C, FIG. 16A).

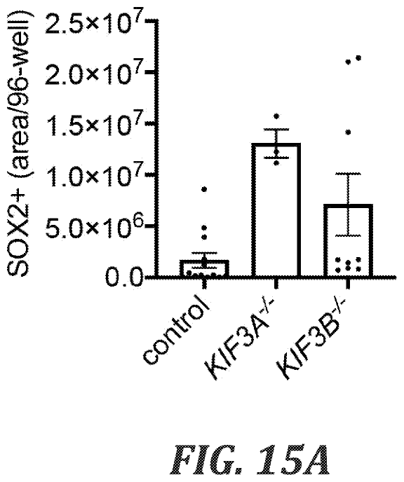

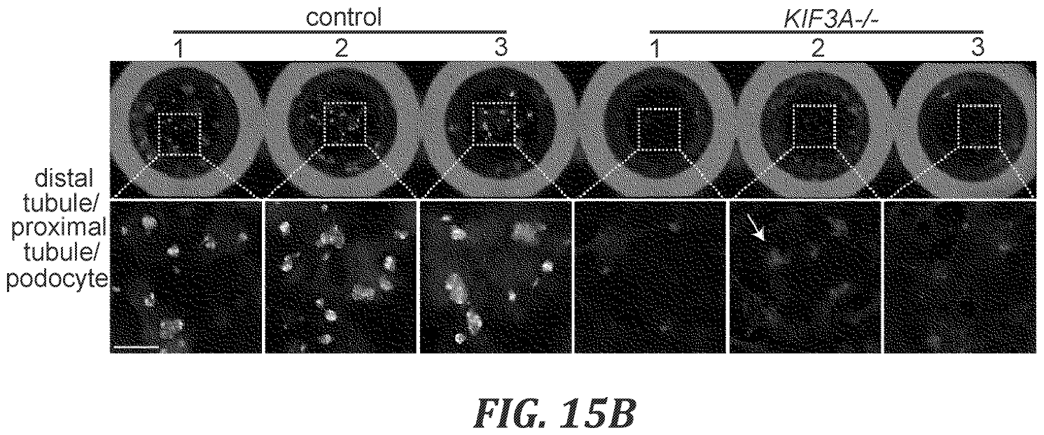

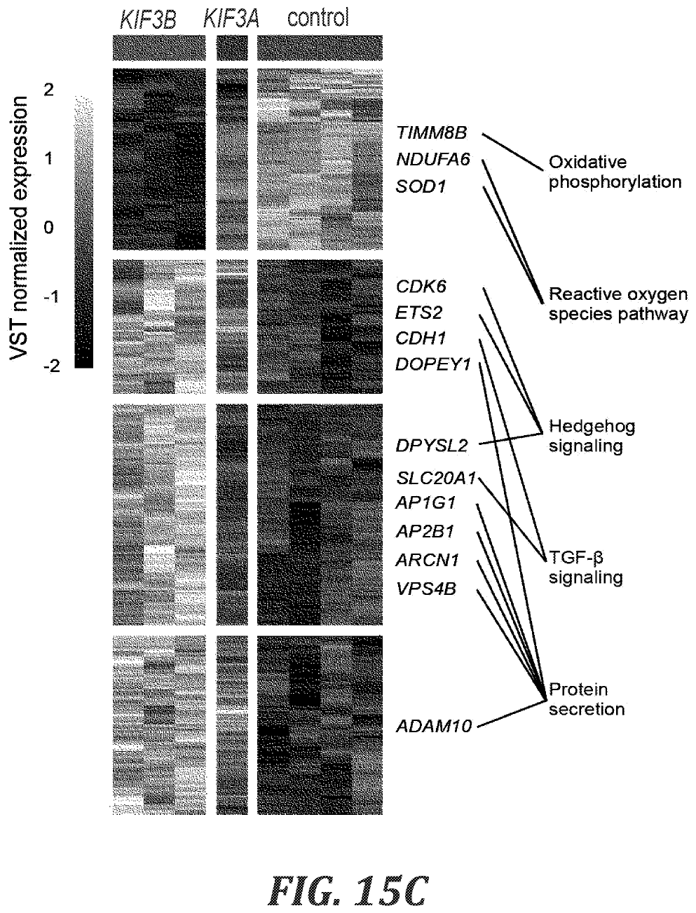

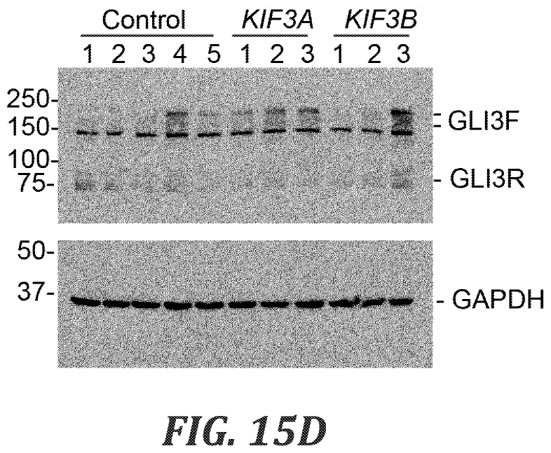

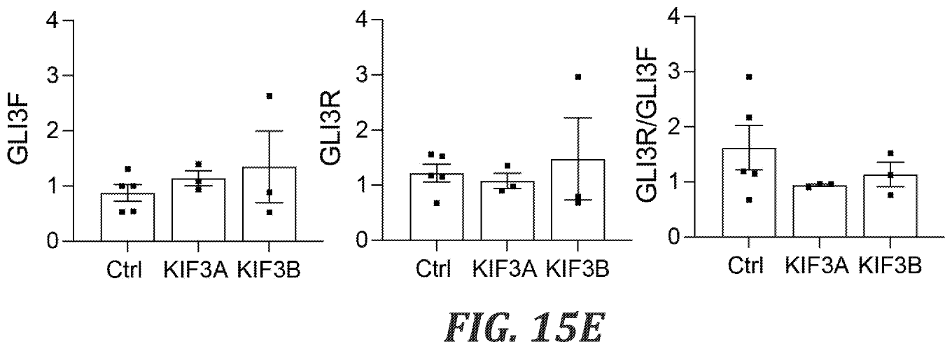

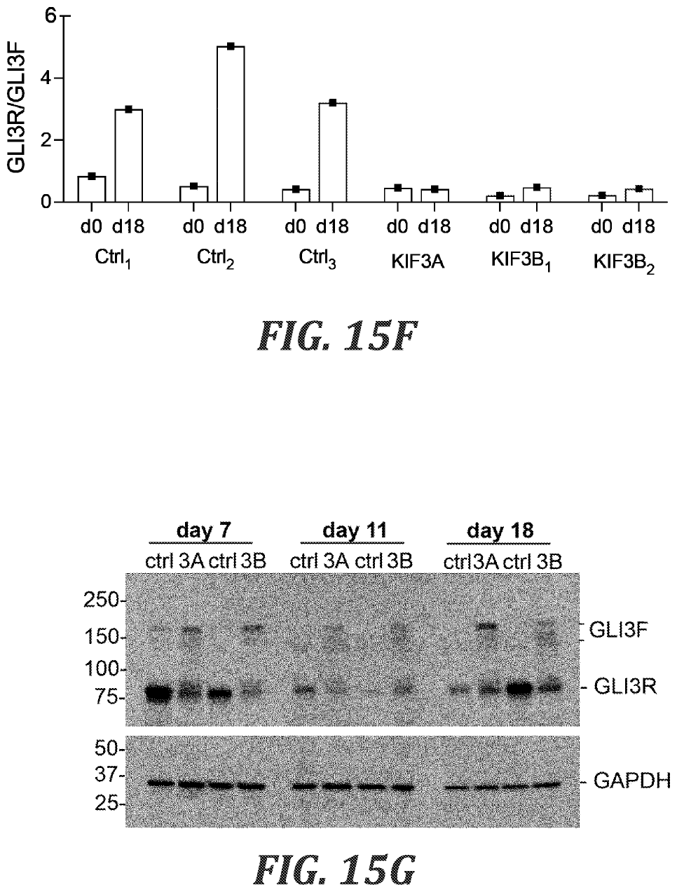

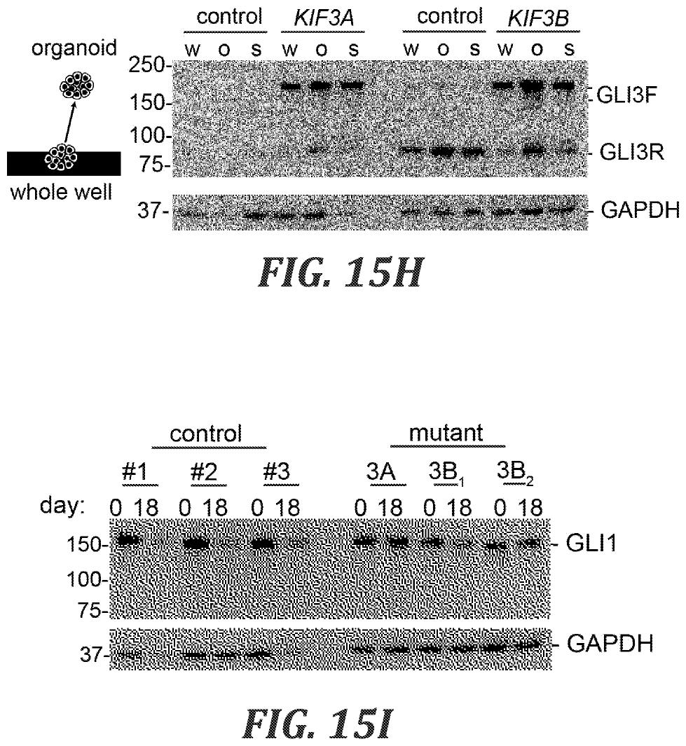

Kidney disease is a common ciliopathy phenotype, manifesting in forms ranging from multicystic dysplastic kidney disease (MCDK) to PKD. Kidney organoids contain distal tubules (ECAD⁺), proximal tubules (LTL⁺), and podocytes (PODXL⁺, NPHS1⁺) in continuous segments. In 96-well plates, control hPSCs formed ˜17 organoids per well, whereas kinesin-2 knockout hPSCs produced ˜2 organoids, indicating a requirement for cilia in the induction of nephron cells (FIG. 4D-F, FIG. 15B).

### A Hedgehog Signaling Switch Promotes Organoid Differentiation

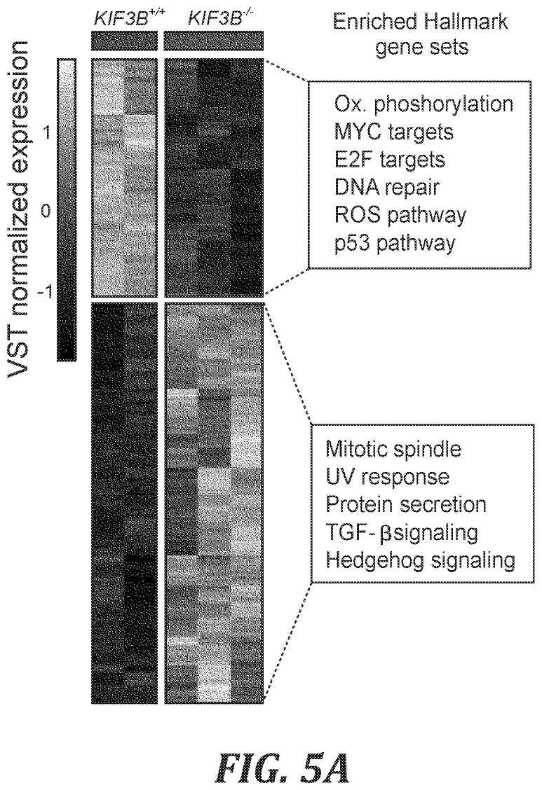

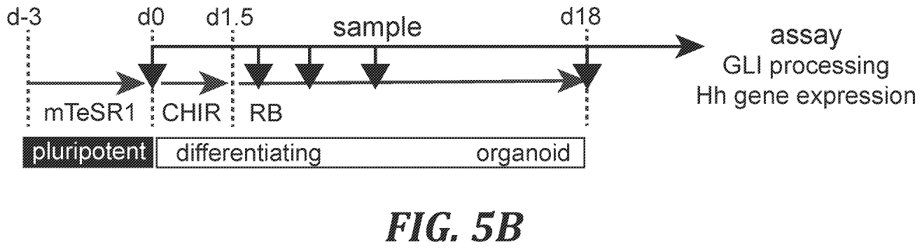

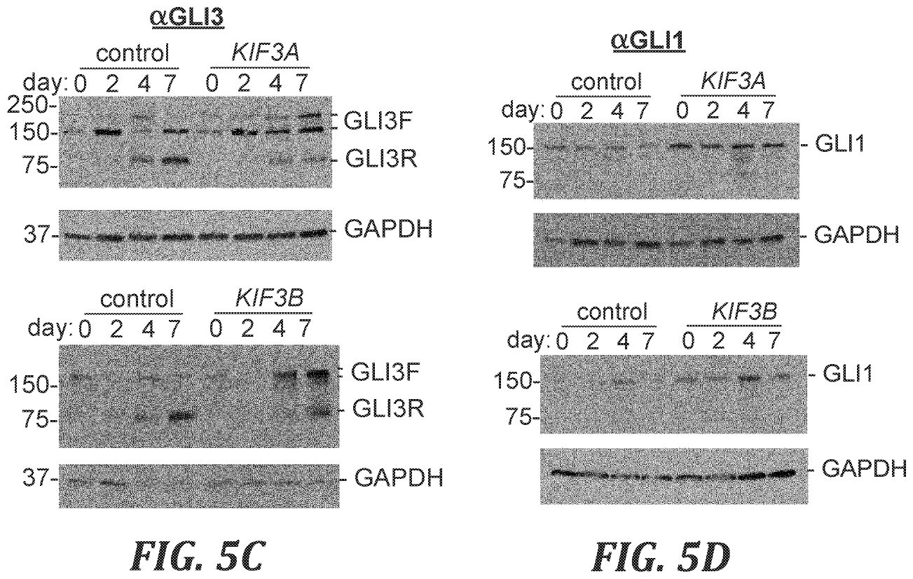

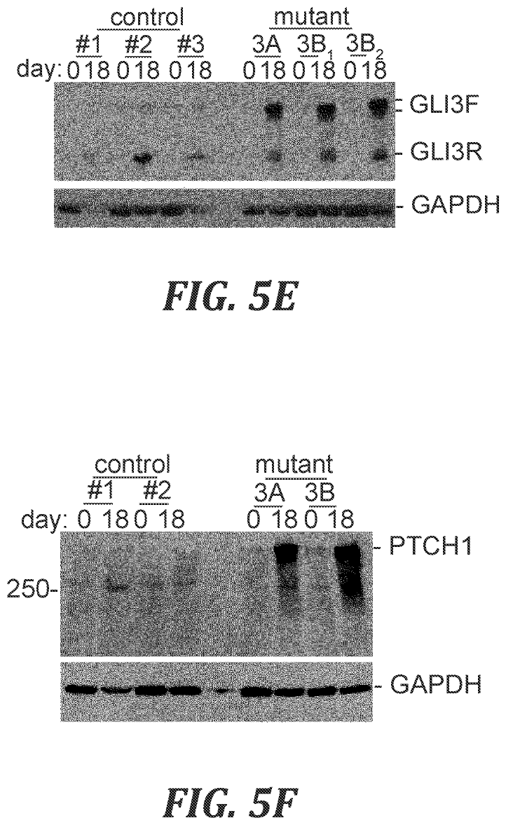

Unbiased RNA-seq analysis identified several differentially expressed gene modules in kinesin-2 knockout hPSCs, including increased activation of hedgehog signaling (FIG. 5A, FIG. 16C). As this pathway is associated with primary cilia, immunoblot analysis of effector GLI proteins and transcriptional targets of hedgehog signaling was conducted during the time course of differentiation (FIG. 5B). The hedgehog effector protein GLI3 can be expressed in full-length forms associated with pathway activation (GLI3F), as well as a shorter, partially processed forms associated with pathway repression (GLI3R). Immunoblots of undifferentiated hPSCs detected two major GLI3F bands of molecular weights ˜ 190 and ˜ 150 kDa, and a minor GLI3R band of molecular weight ˜ 80 kDa (FIG. 5C, FIG. 15D). When quantified by band intensity analysis, the GLI1R:GLI1F ratio was slightly decreased in both KIF3A^{-/-} and KIF3B^{-/-} hPSCs, compared to isogenic controls (FIG. 15E).

In control cells, on days 4-7 of differentiation, GLI3F levels decreased and GLI3R levels increased relative to the undifferentiated state (FIG. 5C). In both KIF3A^{-/-} and KIF3B^{-/-} mutants, however, this shift in GLI3 processing was partially impaired, resulting in lower levels of GLI3R and increased levels of GLI3F, compared to isogenic controls (FIG. 5C). GLI1, a marker of hedgehog pathway activation, was further examined. GLI1 was expressed in undifferentiated hPSCs, increased transiently on day 4 of differentiation, and subsequently decreased on day 7 (FIG. 5D). Compared to controls, both KIF3A^{-/-} and KIF3B^{-/-} mutants expressed higher levels of GLI1, peaking on day 4 with the appearance of lower molecular weight isoforms (FIG. 5D). Increased hedgehog pathway activation was thus detected in kinesin-2 mutants at early stages of differentiation.

Hedgehog signaling was further examined at later stages of differentiation. Day 18 is a time point at which kidney structures in organoids express features of terminal nephron differentiation, such as podocytes. In control cell lines on day 18, the GLI3R:GLI3F ratio was increased ˜6-fold, relative to its value in undifferentiated cells (FIG. 5E). In contrast, both KIF3A^{-/-} and KIF3B^{-/-} cells expressed higher levels of GLI3F, resulting in a GLI3R:GLIF ratio that was only slightly increased on day 18 compared to undifferentiated hPSCs (FIG. 5E, FIG. 15F). Similar GLI3 profiles were observed at intermediate stages in the differentiation process (FIG. 15G). Using a microdissection technique that was previously established to purify nephron structures (Cruz, N. M. et al. Organoid cystogenesis reveals a critical role of microenvironment in human polycystic kidney disease. Nat Mater 16, 1112-1119 (2017); Harder, J. L. et al. Organoid single cell profiling identifies a transcriptional signature of glomerular disease. JCI Insight 4 (2019)), similar defects in both kidney (epithelial) and stromal subcompartments of these organoid cultures were found (FIG. 15H). Thus, the observed defect in GLI3 processing in the mutants was a stable change that was not specific to a particular cell type. Despite sustained reduction in the GLI3R:GLI3F ratio in kinesin-2 knockout mutants, GLI1 levels in these mutants on day 18 were similar to controls, being equivalent or slightly reduced compared to day 0 (FIG. 15I). This result might reflect induction of inhibitors of hedgehog signal transduction such as PTCH1, which is a downstream target of hedgehog signaling. Indeed, PTCH1 was substantially upregulated on day 18 in kinesin-2 knockout mutants, but not in isogenic controls (FIG. 5F). Collectively, these data revealed that loss of kinesin-2 had long-term effects on hedgehog signaling in these cultures.

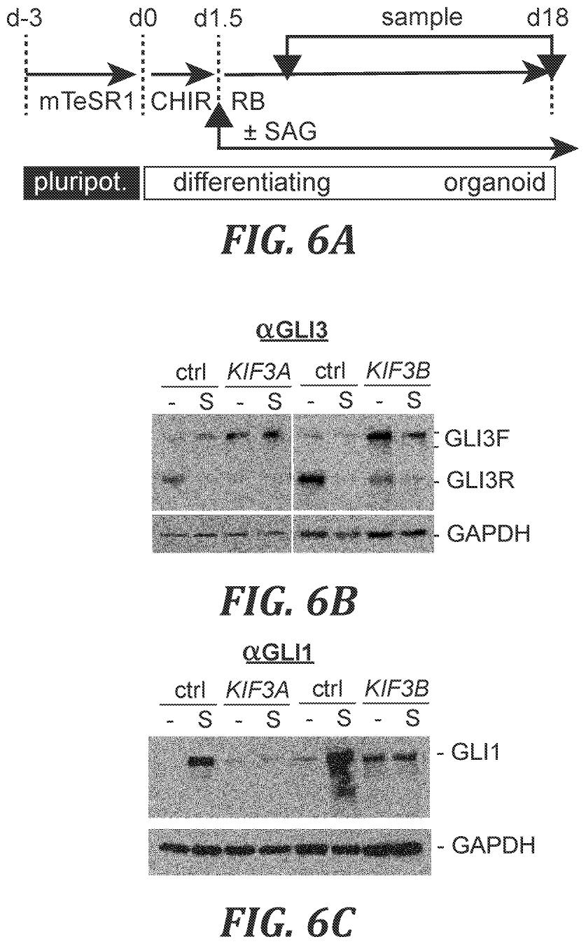

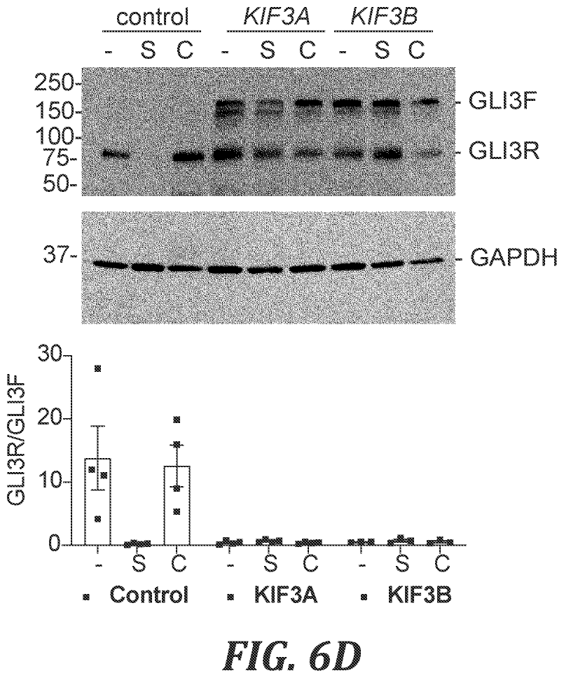

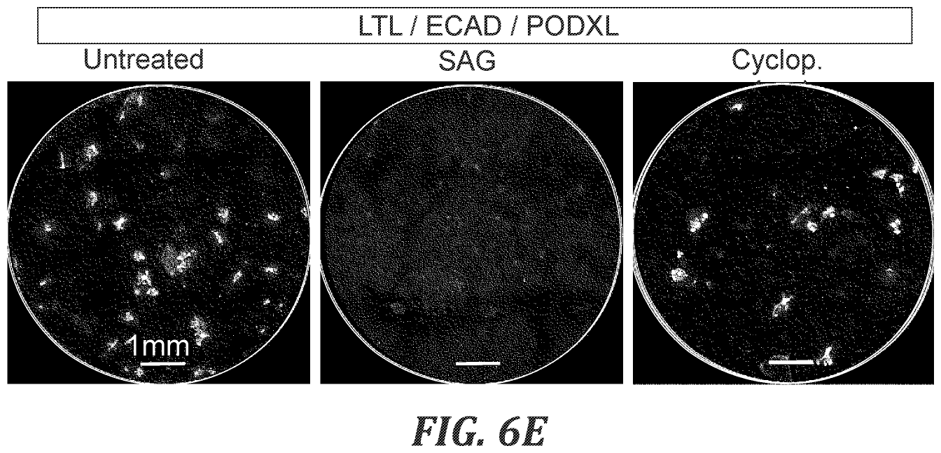

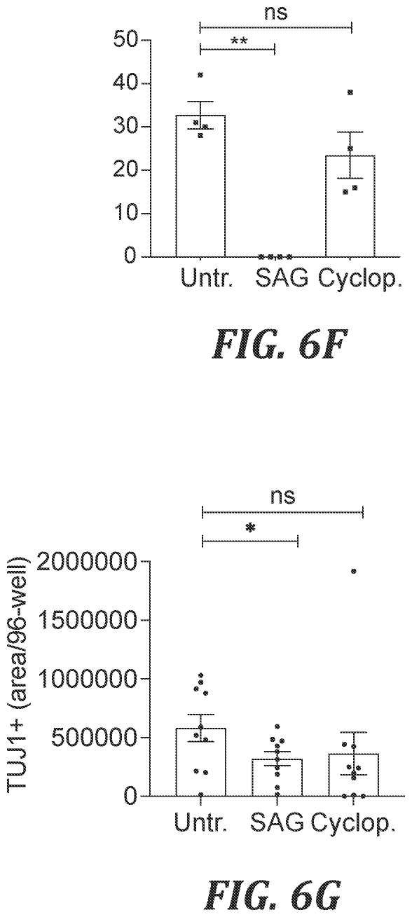

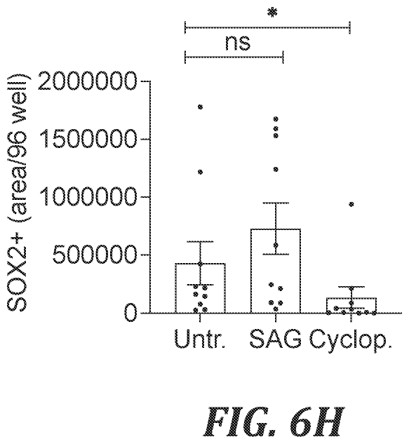

Standard kidney organoid differentiation protocols do not incorporate treatment with small molecules that target hedgehog signaling. To further test hedgehog's role, SAG (smoothened agonist) was added to the culture media, starting on day 1.5 and for the remainder of the differentiation until day 18 (FIG. 6A). By day 4 of differentiation, control cultures treated with SAG expressed reduced levels of GLI3R and increased levels of GLI1, which resembled KIF3A^{-/-} and KIF3B^{-/-} mutants qualitatively, although these changes were more dramatic in SAG-treated control cells than in KIF3A^{-/-} or KIF3B^{-/-} mutants (FIG. 6B-C). Unlike controls, SAG had no detectable effect on GLI3 or GLI1 levels in KIF3A^{-/-} and KIF3B^{-/-} mutants (FIG. 6B-C). By day 18, the prolonged 16.5 day treatment with SAG strongly downregulated GLI3 and upregulated GLI1 in control cultures, whereas treatment with the smoothened antagonist cyclopamine modestly increased GLI3R over the same period (FIG. 6D, FIG. 16A-E). Despite prolonged treatment, KIF3A^{-/-} and KIF3B^{-/-} mutants failed to respond to SAG or cyclopamine in either of these ways (FIG. 6D, FIG. 16A-E).

The effect of hedgehog manipulation with these small molecules was examined on organoid lineage differentiation in the control cultures responsive to these molecules at the molecular level. Kidney organoid differentiation was impaired by SAG, but not cyclopamine (FIG. 6E-F). Neuron differentiation (TUJ1⁺) was significantly suppressed by SAG, but also by cyclopamine, although cyclopamine's effects did not reach statistical significance (FIG. 6G). To understand how SAG and cyclopamine could have similar effects, SOX2⁺ neuronal progenitor cells were examined. SAG increased SOX2⁺ neuronal progenitor cells, whereas cyclopamine had a strong inhibitory effect (FIG. 6G-H). These findings supported the conclusion that modulation of hedgehog signaling was required for proper differentiation in these organoid cultures.

### Kinesin-2 Mutants Form Nephron Organoid Cysts

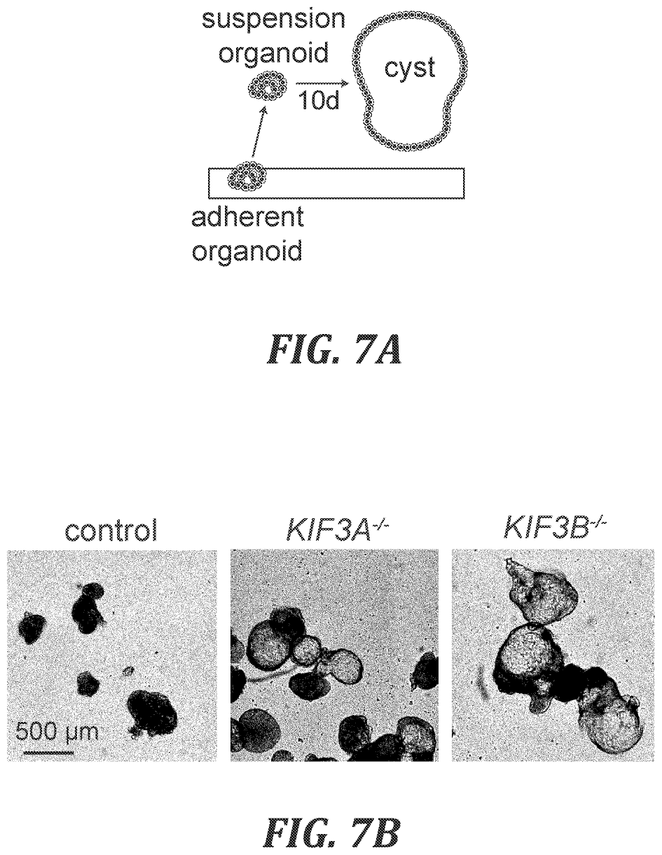

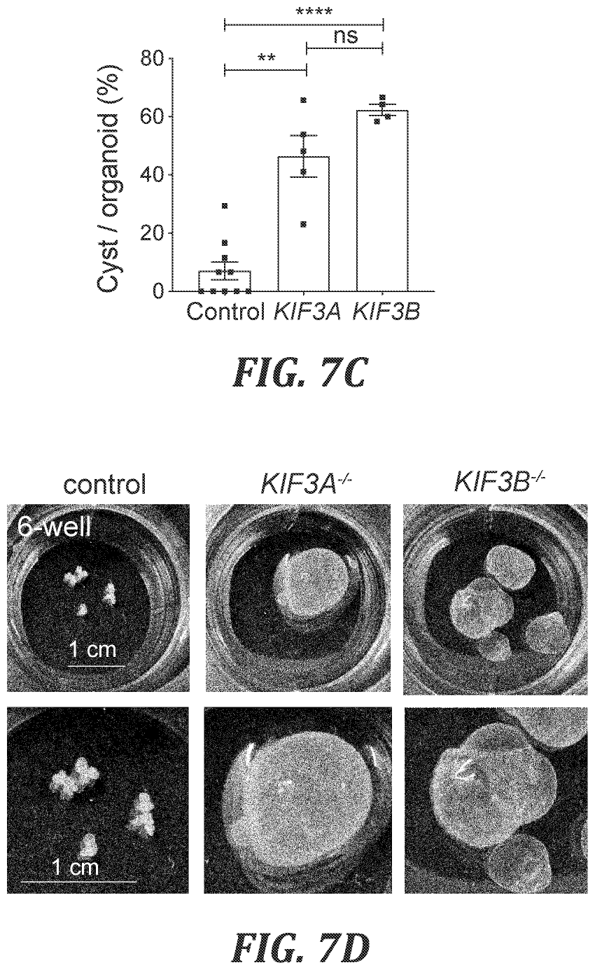

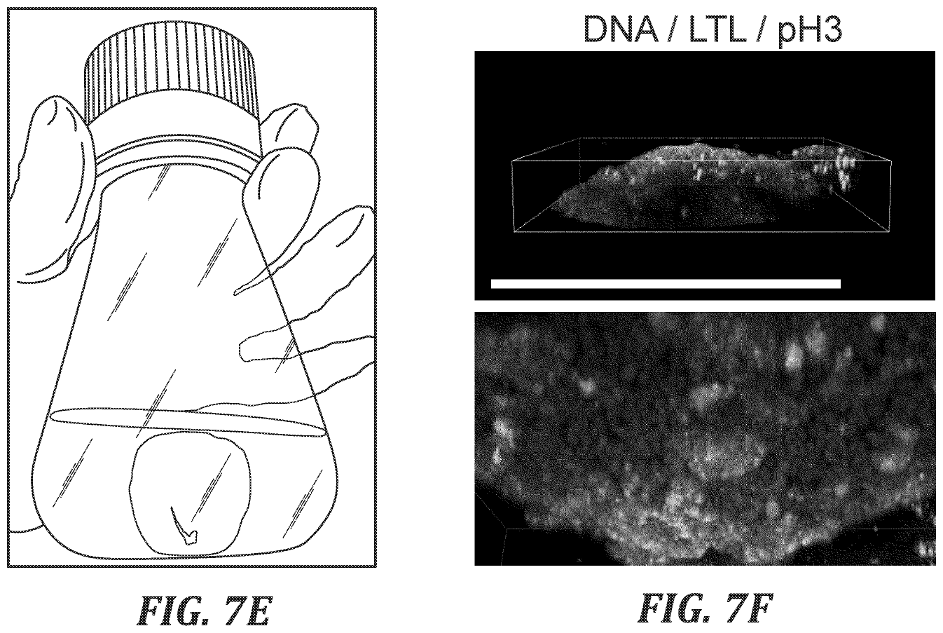

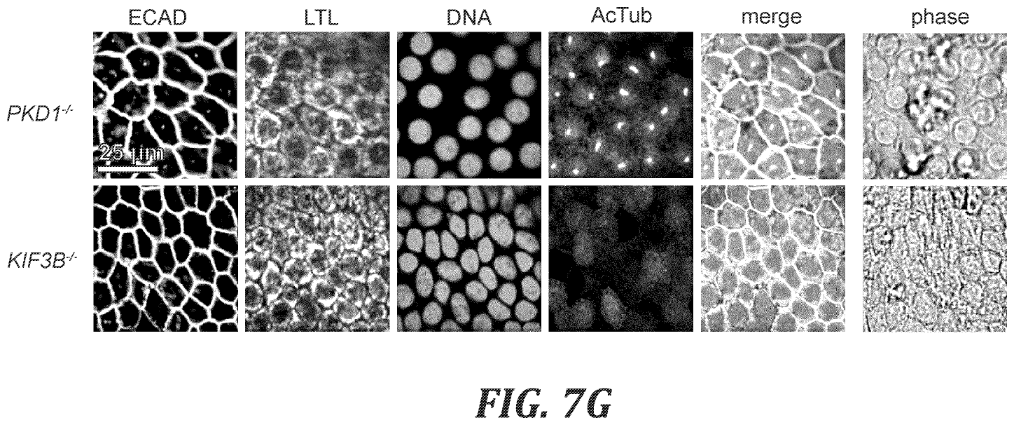

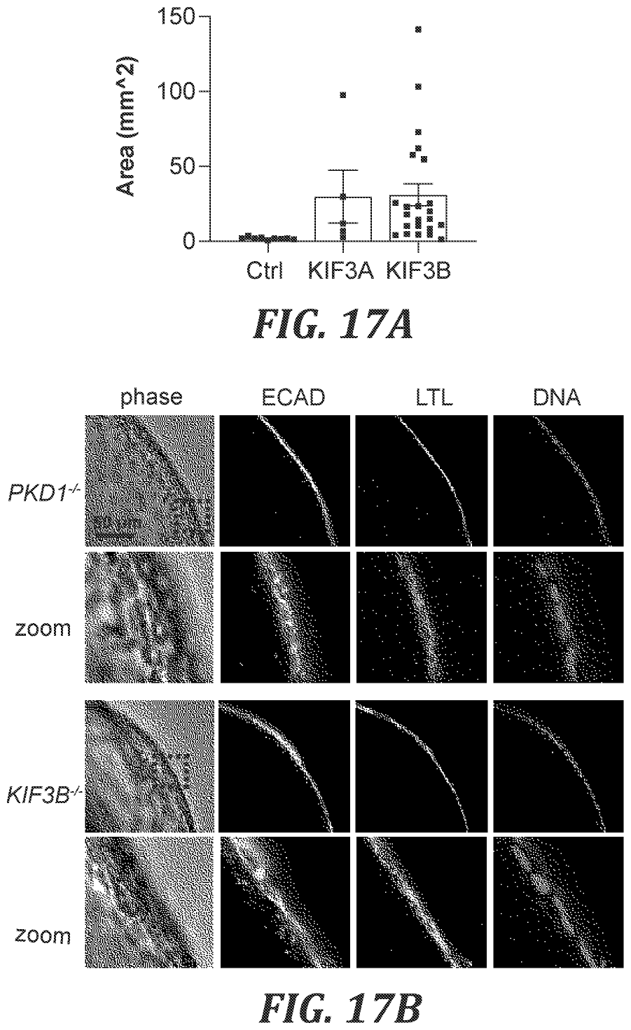

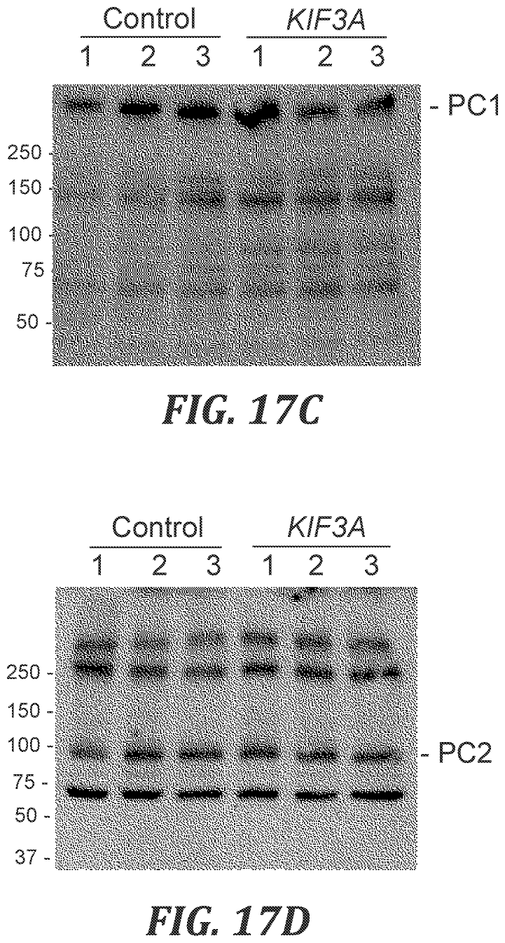

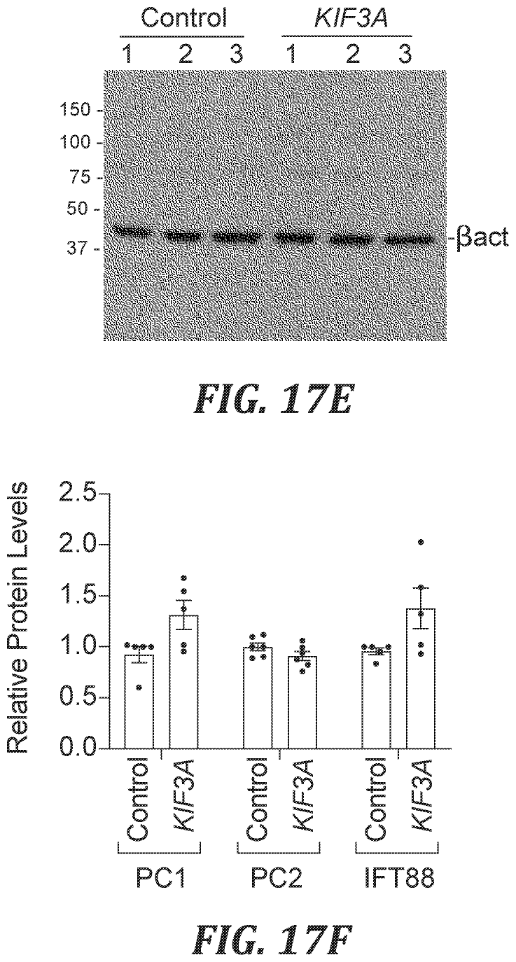

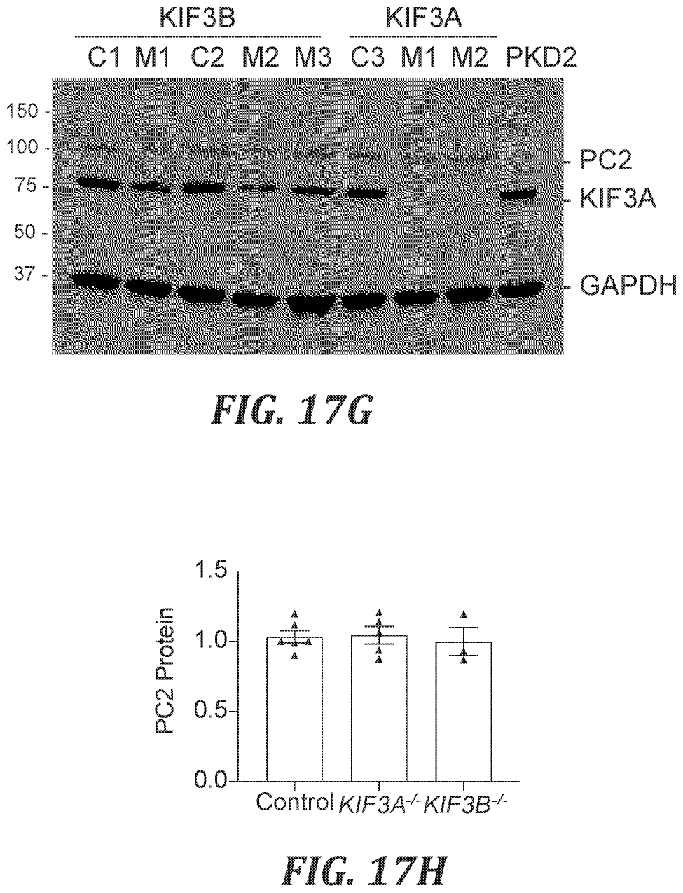

Polycystic kidney disease (PKD) is a common ciliopathy phenotype. Previously, generated kidney organoids were generated from hPSCs with PKD mutations, which form large cysts from tubular epithelial cells, recapitulating the phenotypic hallmark of PKD in vitro (Freedman, B. S. et al. Modelling kidney disease with CRISPR-mutant kidney organoids derived from human pluripotent epiblast spheroids. Nat Commun 6, 8715 (2015); Cruz, N. M. et al. Organoid cystogenesis reveals a critical role of microenvironment in human polycystic kidney disease. Nat Mater 16, 1112-1119 (2017)). Kinesin-2 knockout organoids were tested a PKD phenotype. Although differentiation was inefficient in kinesin-2 knockout lines, a small number of kidney organoids were obtained from mutant hPSCs, which were then transferred into suspension cultures, so that equal numbers of organoids from controls and mutants could be compared (FIG. 7A). Under these conditions, ˜ 50% of kinesin-2 knockout organoids formed large, translucent cysts after two weeks, whereas isogenic controls rarely formed cysts (FIG. 7B-C). Kinesin-2 knockout cysts arose spontaneously, without any forskolin addition. These cysts grew dramatically in size over several months, reaching diameters of >1 cm, whereas isogenic controls were limited to mm diameters (FIG. 7D). In extended cultures, cysts could reach several centimeters in diameter and needed to be transferred into 200 mL flasks to accommodate their large size (FIG. 7E, FIG. 17A). Cysts stained positive for the proximal tubular marker LTL in patches and contained a thin layer of pH3⁺ cells surrounding a hollow lumen, indicating they comprised proliferating kidney tubular epithelial cells (FIG. 7F). LTL⁺ cyst-lining epithelial cells co-expressed the distal tubular marker ECAD, similar to PKD1^{-/-} organoid cysts (FIG. 7G, FIG. 17B). PKD1^{-/-} cysts, however, elaborated abundant primary cilia, which were absent in kinesin-2 knockout cysts (FIG. 7G). Conversely, no substantive difference was detected in the levels of the PKD1 protein product, polycystin-1 (PC1), or the PKD2 protein product, polycystin-2 (PC2), in whole cell lysates from kinesin-2 knockout hPSCs (FIG. 17C-H). This is distinct from the PC1 and PC2 knockout lines that were previously described, in which PC1 expression is lost even in PKD2^{-/-} cells (Freedman, B. S. et al. Modelling kidney disease with CRISPR-mutant kidney organoids derived from human pluripotent epiblast spheroids. Nat Commun 6, 8715 (2015)).

Collectively, these experiments revealed two distinct phenotypes in kinesin-2 knockout kidney organoids: decreased nephrogenesis, and increased cystogenesis among the nephron structures that do form. This differs from the pure PKD phenotype, in which cysts form but nephrogenesis occurs normally. Nephrogenesis and cystogenesis may be regulated, respectively, by the hedgehog and PKD pathways, both of which likely require IFT for signal transduction and would be disrupted simultaneously in kinesin-2 knockout hPSCs. Reduced nephrogenesis would be expected to have a dominant effect in the context of PKD, which might explain the smaller size of kidneys in Kif3a-Pkd2 double-mutant mice compared to Pkd2 single mutants.

### Human Cilia Constitutively Release Extracellular Vesicles Containing Disease-Associated Proteins

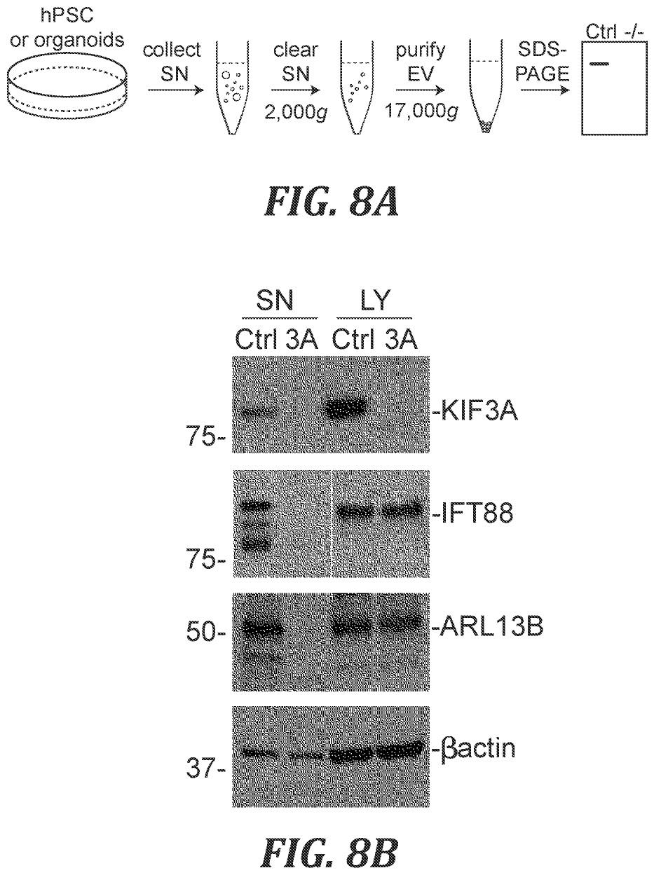

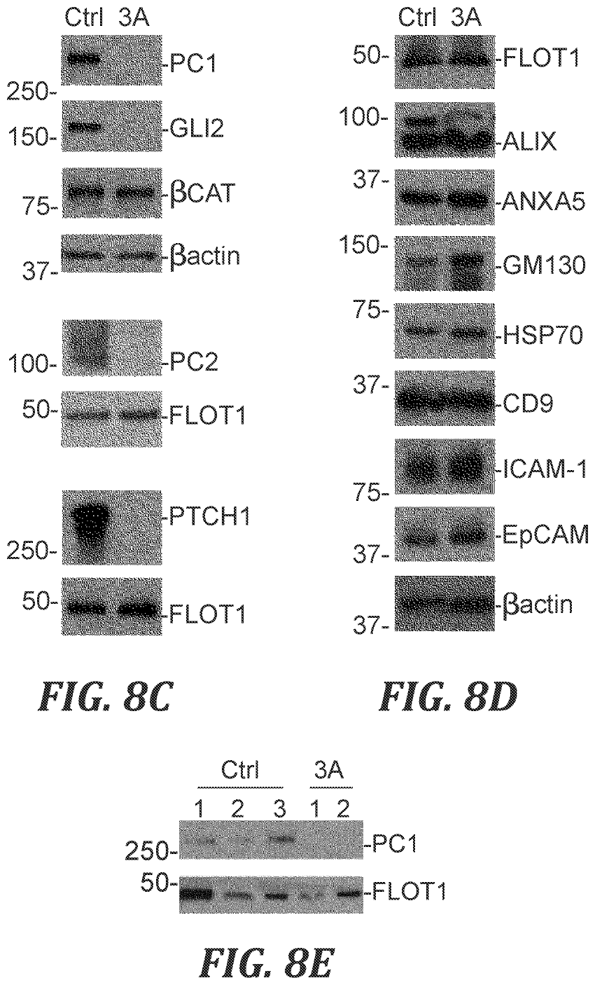

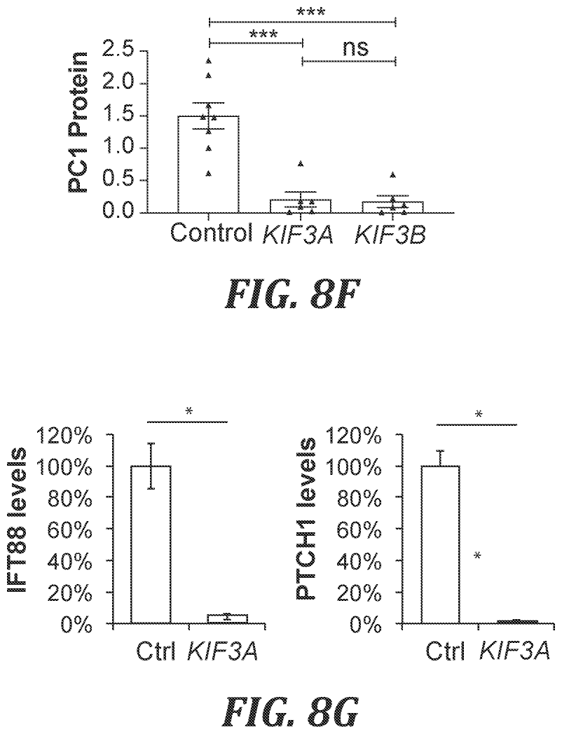

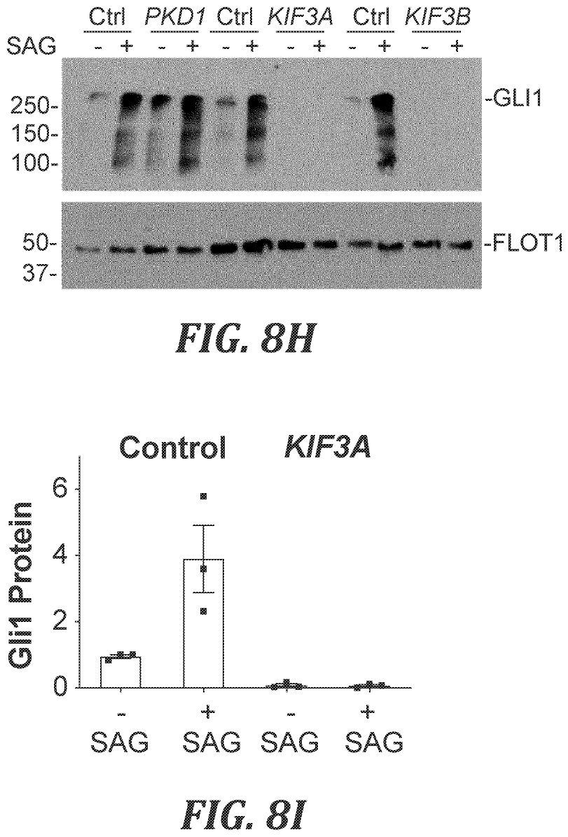

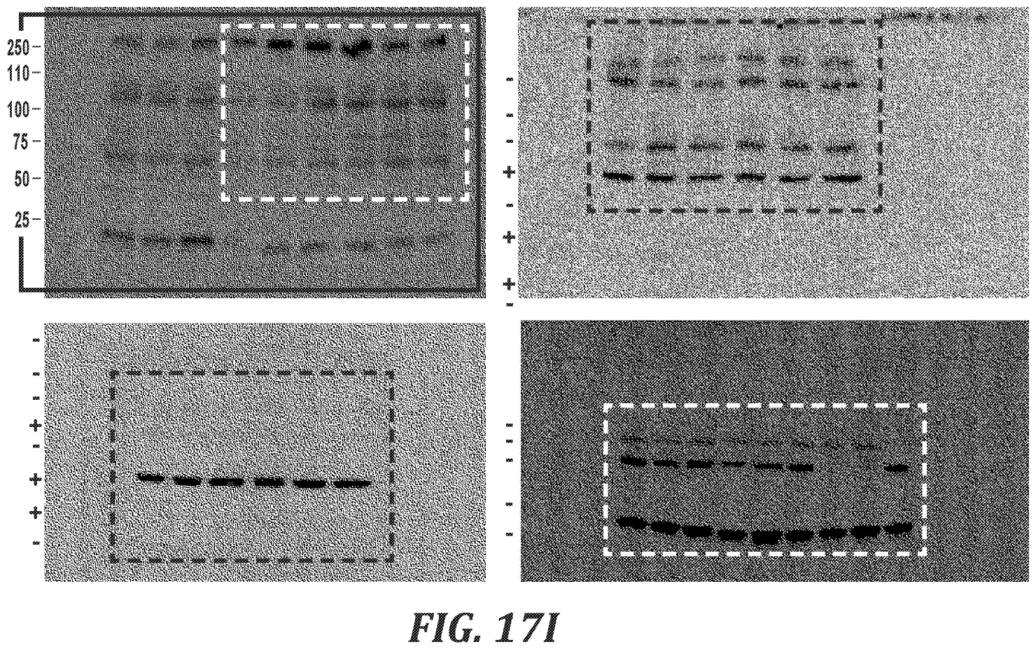

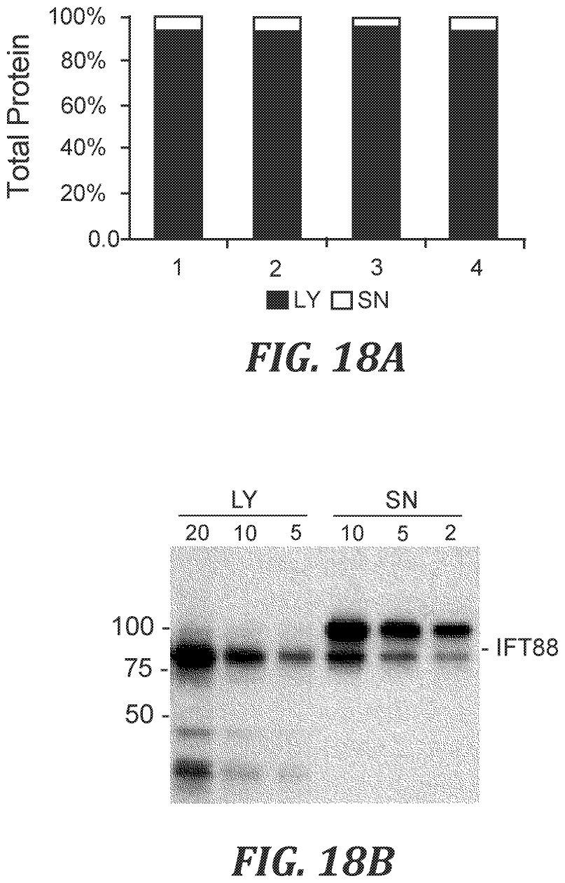

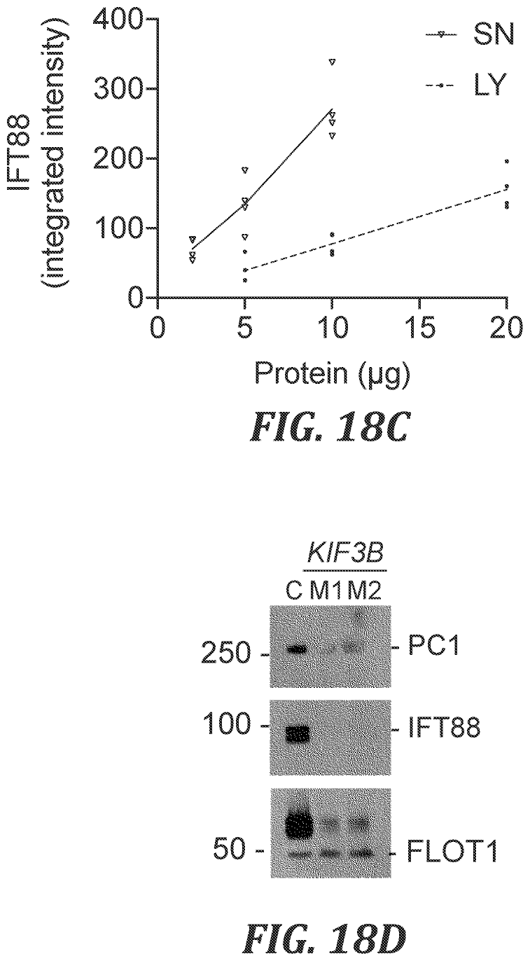

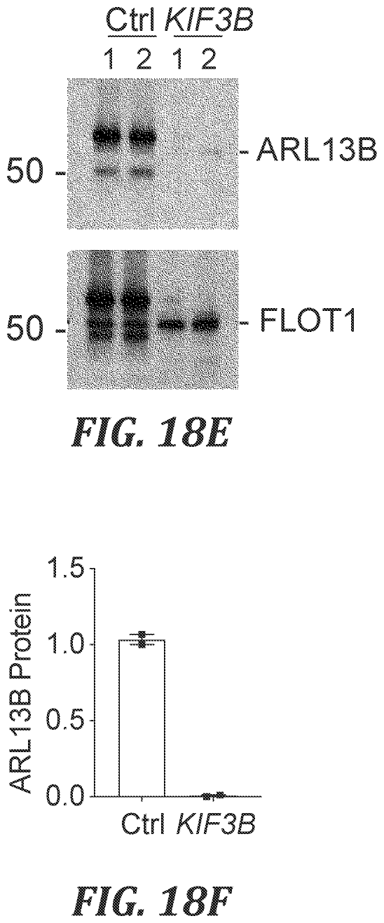

To further determine the mechanistic consequences of kinesin-2 loss, a direct biochemical assay was developed to measure the expression of ciliopathy-linked proteins in both whole cell lysates and extracellular vesicles (EV) (FIG. 8A). Cilia have been suggested to release EV, but these have been difficult to detect in immortalized mammalian cell lines without starvation, and even under optimized conditions do not appear to contain detectable quantities of many key membrane proteins. EV protein amounted to only ˜ 6% of total protein (FIG. 18A). Per mg protein, however, the relatively small EV fraction was enriched ˜ 5-fold for IFT88, an intracellular protein involved in ciliary transport, relative to whole cell lysates (FIG. 18B-C).

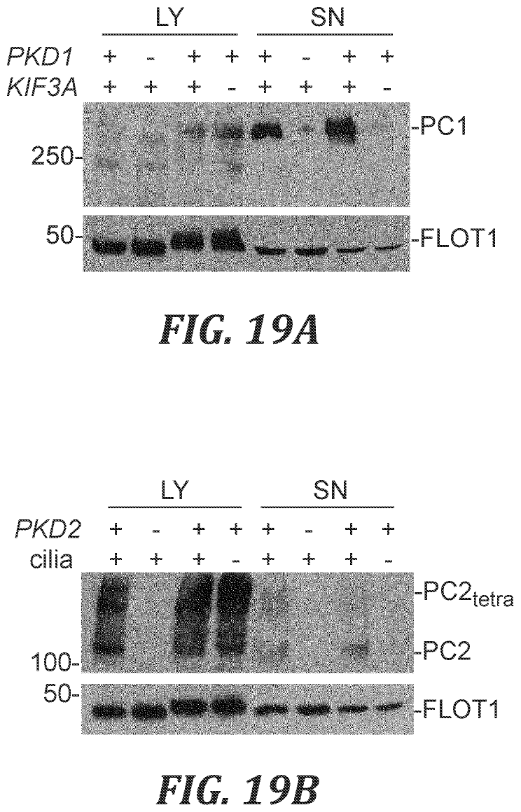

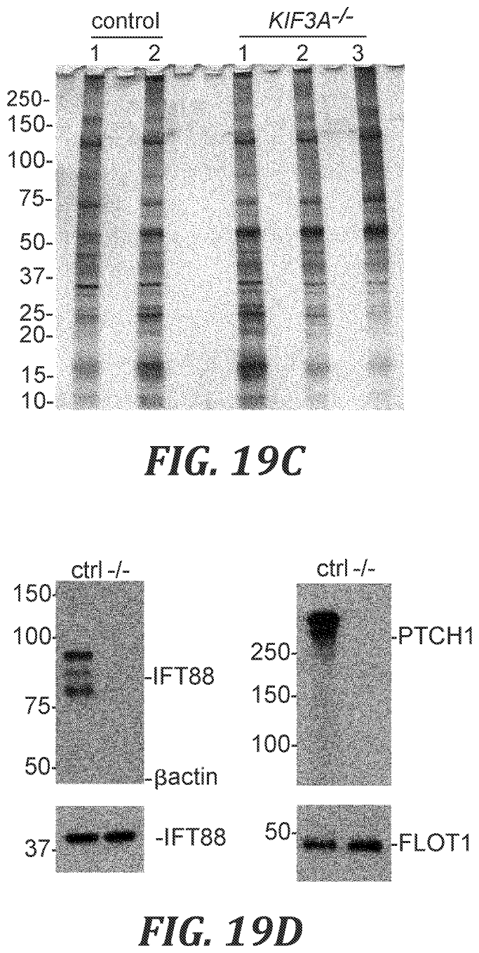

In kinesin-2 knockout hPSCs, as expected, the kinesin-2 subunit targeted by genome editing (KIF3A or KIF3B) was absent in both lysates and supernatants as a control (FIG. 8B). In contrast, components of the IFT (ARL13B, IFT88), hedgehog (PTCH1, GLI2), and PKD/polycystin (PC1, PC2) pathways, as well as the non-targeted kinesin-2 subunit, were expressed at normal levels in whole cell lysates of kinesin-2 knockout hPSCs, but were dramatically reduced in EV of kinesin-2 knockout hPSCs (FIG. 8B-C, FIG. 18D-F). The magnitude of PC1 and PC2 loss from EV in kinesin-2 knockout hPSCs was similar to that observed in PKD1^{-/-} or PKD2^{-/-} hPSCs (FIG. 19A-B) (Freedman, B. S. et al. Modelling kidney disease with CRISPR-mutant kidney organoids derived from human pluripotent epiblast spheroids. Nat Commun 6, 8715 (2015). Compared to PC2, the proportion of cellular PC1 was highly enriched in EV, indicating a stronger propensity of PC1 to exit the cell (FIG. 19A-B). This is significant, because PC1 is difficult to detect intracellularly, whereas PC2 contains an endoplasmic reticulum retention signal and is more readily detected inside cells.

The inability of kinesin-2 knockout hPSCs to release these proteins did not reflect a general inability to release EV, as non-IFT cargoes, including β-actin, HSP70, and flotillin, were efficiently released by kinesin-2 knockout hPSCs, and no differences in total protein level or banding pattern were detected (FIG. 8B-D, FIG. 19C). Loss of extracellular PC1 was also observed in EV of kidney organoids derived from kinesin-2 knockout hPSCs (FIG. 8E). Quantification indicated an 8-fold reduction in PC1 levels, and a 20-fold reduction in ARL13B and PTCH1 levels, in kinesin-2 knockout supernatants, compared to controls (FIG. 8F-G, FIG. 18D-F, and FIG. 19D). In kinesin-2 knockouts, GLI1 was undetectable in EV, even after SAG stimulation, which contrasted with control or PKD1^{-/-} hPSCs, in which GLI1 was detected in EV at steady-state and increased after SAG stimulation (FIG. 8H-I). These findings revealed that EV release in kinesin-2 knockout hPSCs is a robust, constitutive process under non-starvation conditions, enabling the sensitive detection of membrane proteins associated with ciliopathy phenotypes.

A new general tool and a collection of KIF3A^{-/-} and KIF3B^{-/-} hPSCs has been established to elucidate the developmental and disease-related functions of cilia in human somatic cell types and tissues at different stages of maturation. Despite proposed roles of cilia in cell cycle regulation and signaling, both KIF3A^{-/-} and KIF3B^{-/-} hPSCs are stably pluripotent and self-renewing, providing an ideal starting point for differentiation experiments into diverse lineages without the complication of embryonic lethality. Phenotypes of defective neurogenesis and nephrogenesis, as well as cystogenesis in the nephron structures that do form, manifest in complex tissues and organoids, but are not observed in undifferentiated hPSCs or simpler EBs in vitro, suggesting that cilia play a critical role in the higher-order organization of complex tissue architecture, for instance by coordinating the migration of scattered cell types. The fact that such tissue-scale phenotypes can be reconstituted in a relatively simple organoid culture in vitro demonstrates that they are driven by cell intrinsic processes, rather than complex interactions between organ systems or systemic effects that can only occur in a living organism.

KIF3A and KIF3B form the heterodimeric motor subunits of kinesin-2. KIF3B has been studied less extensively than KIF3A, shows greater genetic variation in humans, and can under certain circumstances be replaced in kinesin-2 by KIF3C. In the inventor's experiments studying hPSC differentiation, KIF3A^{-/-} and KIF3B^{-/-} hPSCs have very similar phenotypes, indicating that the effects are specific to kinesin-2, and that both members of the complex are necessary for function. This is consistent with studies of KIF3A and KIF3B knockout mice, which show very similar phenotypes to one another, dying in mid-gestation with various malformations including open neural tubes (Marszalek, J. R., Ruiz-Lozano, P., Roberts, E., Chien, K. R. & Goldstein, L. S. Situs inversus and embryonic ciliary morphogenesis defects in mouse mutants lacking the KIF3A subunit of kinesin-II. Proc Natl Acad Sci USA 96, 5043-5048 (1999); Nonaka, S. et al. Randomization of left-right asymmetry due to loss of nodal cilia generating leftward flow of extraembryonic fluid in mice lacking KIF3B motor protein. Cell 95, 829-837 (1998)). Further study in diverse lineages may yet reveal subtle differences between the functions of KIF3A and KIF3B, and the full extent to which each of these is required for cilia formation. In addition to its effects on cilia, kinesin-2 can also play compounding non-ciliary roles, which we have not ruled out (Scholey, J. M. Kinesin-2: a family of heterotrimeric and homodimeric motors with diverse intracellular transport functions. Annu Rev Cell Dev Biol 29, 443-469 (2013)). KIF3A^{-/-} and KIF3B^{-/-} hPSCs can be extensively passaged and expanded, establishing a long-term resource for investigating these topics.

The ability to vary differentiation and growth conditions in KIF3A^{-/-} and KIF3B^{-/-} hPSCs in vitro represents a major advantage of this system for elucidating functional mechanisms. For example, both KIF3A^{-/-} and KIF3B^{-/-} knockout cells differentiate normally in EBs but are impaired in teratomas and organoids. Analysis of organoid cultures during the process of differentiation reveals a critical switch in hedgehog signaling from activation to repression, which depends upon kinesin-2 for proper modulation. In these cultures, differentiation is induced by transient exposure to a kinase inhibitor immediately upon withdrawal from pluripotent stem cell maintenance media (Freedman, B. S. et al. Modelling kidney disease with CRISPR-mutant kidney organoids derived from human pluripotent epiblast spheroids. Nat Commun 6, 8715 (2015)). As described in this Example, this step produces a transient increase in hedgehog pathway activation, while simultaneously triggering a more permanent switch to hedgehog repression. This switch and its consequences would not be discernable in somatic cells that do not undergo differentiation, nor has it been suggested in previous analyses of kidney organoids (Glass, N. R. et al. Multivariate patterning of human pluripotent cells under perfusion reveals critical roles of induced paracrine factors in kidney organoid development. Sci Adv 6, eaaw2746 (2020); Wu, H. et al. Comparative Analysis and Refinement of Human PSC-Derived Kidney Organoid Differentiation with Single-Cell Transcriptomics. Cell Stem Cell 23, 869-881 e868 (2018); Subramanian, A. et al. Single cell census of human kidney organoids shows reproducibility and diminished off-target cells after transplantation. Nat Commun 10, 5462 (2019)). There is genetic evidence that hedgehog repression promotes nephrogenesis in the mouse, although this role has been largely overshadowed by studies of Pallister Hall syndrome, in which GLI3R-mediated hedgehog repression is maladaptive for kidney development, and the role of cilia in this process remains poorly understood (Chi, L. et al. Kif3a controls murine nephron number via GLI3 repressor, cell survival, and gene expression in a lineage-specific manner. PLoS One 8, e65448 (2013); D'Cruz, R., Stronks, K., Rowan, C. J. & Rosenblum, N. D. Lineage-specific roles of hedgehog-GLI signaling during mammalian kidney development. Pediatr Nephrol 35, 725-731 (2020)).

The mechanism whereby GLI1 expression is increased transiently may relate to a form of negative feedback involving PTCH1, which inhibits Smoothened but is simultaneously a downstream target of hedgehog pathway activation. Increased PTCH1 in kinesin-2 knockout mutants on day 18 of differentiation thus supports sustained hedgehog activation in these mutants, and also provides a possible explanation for the reduction of GLI1 over time. While distinct from control cells, kinesin-2 knockout mutants also exhibited clear changes in GLI3 processing and in the expression levels of downstream hedgehog target genes (GLI1 and PTCH1) during the time course of differentiation. Thus, some capacity to execute hedgehog signaling responses is retained without a cilium. Kinesin-2 knockout mutants were, however, numbed in their ability to respond to SAG or cyclopamine. Based on these findings, distinct activators or inhibitors of hedgehog signaling may have different dependencies on cilia.

Long-term supplementation of our organoid differentiation protocol with hedgehog agonist (SAG) or antagonist (cyclopamine) was detrimental to neuronal differentiation. In other neuronal differentiation protocols, hedgehog agonists have been used to promote neuronal differentiation (Mak, S. K. et al. Small molecules greatly improve conversion of human-induced pluripotent stem cells to the neuronal lineage. Stem Cells Int 2012, 140427 (2012); Kriks, S. et al. Dopamine neurons derived from human ES cells efficiently engraft in animal models of Parkinson's disease. Nature 480, 547-551 (2011)). The inventor did observe an increase in neuronal progenitor cells in cultures treated with SAG, which appeared to come at the expense of differentiation into mature TUJ1+ neurons. In contrast to SAG, cyclopamine had a generally detrimental effect on the differentiation of both neuronal progenitor cells and neurons. The detrimental effect of SAG on neuronal differentiation in our protocol, compared to other protocols, can be due to its prolonged treatment in these experiments, or its use in addition to high doses of a kinase inhibitor that is already sufficient to transiently activate hedgehog signaling. A balance of hedgehog repression and activation may be necessary for optimal neuronal differentiation, as well as for renal cell fates. The inventor notes that in addition to hedgehog, the RNA-seq analysis suggests that other signaling pathways such as TGF-b may be dysregulated in hPSCs devoid of cilia, which could potentially affect differentiation or disease phenotypes in these cultures (Cruz, N. M. et al. Organoid cystogenesis reveals a critical role of microenvironment in human polycystic kidney disease. Nat Mater 16, 1112-1119 (2017); Morizane, R. et al. Nephron organoids derived from human pluripotent stem cells model kidney development and injury. Nat Biotechnol 33, 1193-1200 (2015)).

Beyond effects on differentiation, generation of KIF3A^{-/-} and KIF3B^{-/-} kidney organoids enables the deconvolution of the PKD phenotype from the nephrogenesis defect, with implications for understanding the paradoxical effects of IFT knockout in the context of PKD. In contrast to polycystin knockouts, both KIF3A^{-/-} and KIF3B^{-/-} hPSCs express normal levels of PC1 and PC2 in whole cell lysates but lack cilia. Using KIF3A^{-/-} and KIF3B^{-/-} hPSCs, a robust requirement for kinesin-2 in the release of ciliopathy-related signaling molecules from cells into the surrounding microenvironment, including PC1 was identified. This is consistent with detailed cell biology analyses demonstrating release of EV from ciliary tips containing specific cargoes, which are distinct from EV released from non-ciliary sites on the plasma membrane (Phua, S. C. et al. Dynamic Remodeling of Membrane Composition Drives Cell Cycle through Primary Cilia Excision. Cell 168, 264-279 e215 (2017); Nager, A. R. et al. An Actin Network Dispatches Ciliary GPCRs into Extracellular Vesicles to Modulate Signaling. Cell 168, 252-263 e214 (2017)). As kinesin-2 knockout organoids show defects in both GLI3 processing and EV release, it cannot yet clearly be distinguished which of these mechanisms is causative for cystogenesis. Deficiencies in hedgehog signaling are not evident in polycystin knockouts, suggesting that the PKD pathway is separable and independent from hedgehog, but shares the requirement for cilia as a signaling organizing center. EV release may serve to dispatch extracellular signals packaged in EV, similar to specialized neuronal cilia of nematode worms, or alternatively to dispose of ciliary material in order to trim ciliary length, as suggested in studies of immortalized mouse kidney cells (Phua, S. C. et al. Dynamic Remodeling of Membrane Composition Drives Cell Cycle through Primary Cilia Excision. Cell 168, 264-279 e215 (2017); Nager, A. R. et al. An Actin Network Dispatches Ciliary GPCRs into Extracellular Vesicles to Modulate Signaling. Cell 168, 252-263 e214 (2017).

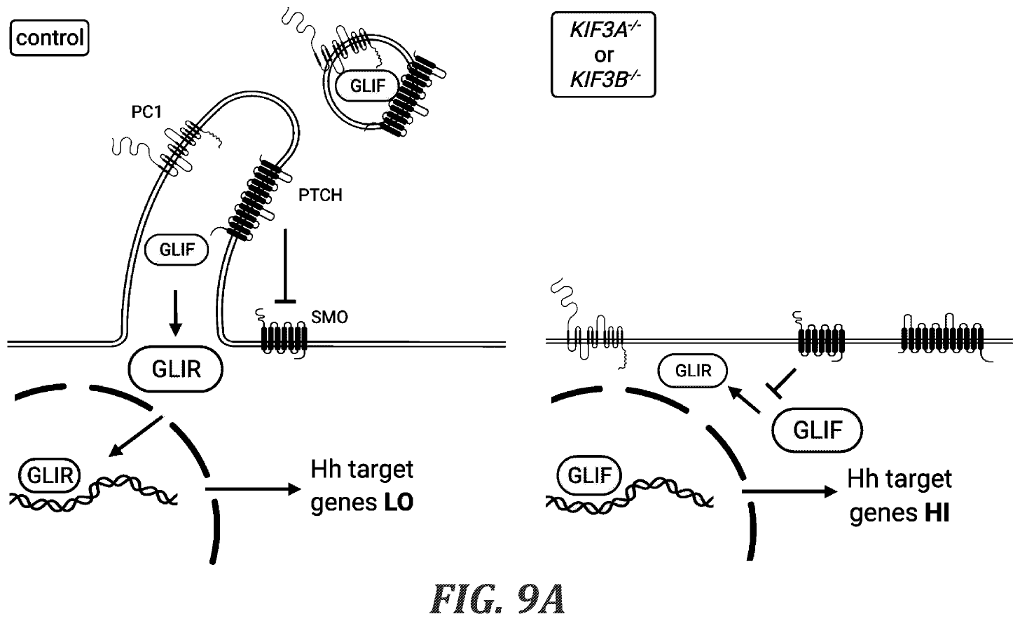

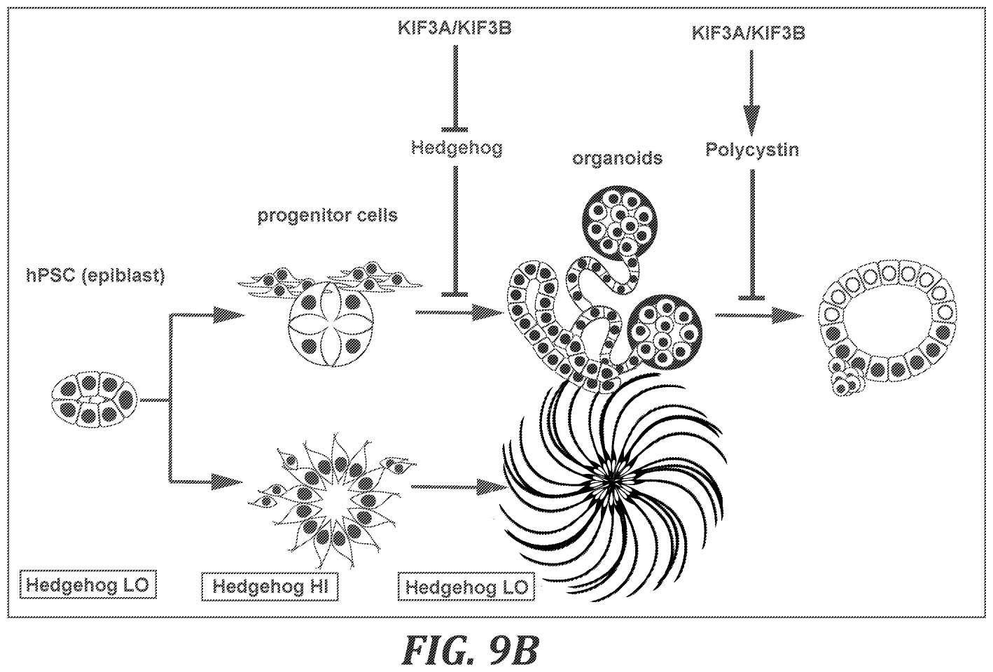

Collectively, the data disclosed in this Example suggests a model wherein human cilia function as both a protected domain for organizing signaling functions, and constitutive secretory organelles that release EV packets containing specific combinations of signaling molecules (FIG. 9A). In mutants, loss of hedgehog responsiveness in stem cells inhibits terminal differentiation, while loss of PKD activity in differentiated cells leads to cystogenesis (FIG. 9B). Ciliary function is therefore cell type- and context-dependent and may be increased in mammalian developmental lineages poised for embryonic differentiation.

In conclusion, disruption of IFT in hPSCs establishes a human genetic model of cilia function, providing critical insight into this enigmatic organelle. Using this new tool, it is now possible to perform phenotypic screens and mechanistic studies to reveal the functions of human cilia and test interventional strategies for ciliopathy syndromes or cilia-dependent cancers. This is the first time that human tissues and organoids have been generated that lack cilia and the first time comparing human tissues and organoids lacking cilia to isogenic structures with cilia. A major strength of this system is its ability to reveal and de-convolve phenotypes under modular differentiation conditions, without the complication of embryonic lethality. Biochemical analysis in this system further indicates a critical role for cilia in organoid differentiation, and in the constitutive extracellular release of key signaling molecules. As these studies are performed in human stem cells at the earliest stages of embryonic development, understanding and controlling cilia may provide a path to guiding and improving regenerative therapies.

Generation of Gene-Edited hPSCs

Plasmids encoding guide RNA targeting KIF3A (5′-CATATGGACAAACCGGAAC-3′) (SEQ ID NO: 1) or KIF3B (5′-TTCGCTGTCGGCCCATGAA-3′ (SEQ ID NO: 2) or 5′-TACACCATGGAAGGAATCCG-3′) (SEQ IS NO: 3) and GFP-tagged Cas9 were co-transfected transiently into a subclone of WA09 (H9) ESCs (WiCell) or WTC11 iPSC (Coriell #25256) using Lipofectamine Stem transfection reagent (Thermo Fisher). GFP⁺ cells were isolated using a FACS Aria cell sorter and plated at low density in 6-well dishes coated with 1% Geltrex (Thermo Fisher). Clones were manually picked with a 10 μL pipet and transferred to a 96-well plate. DNA was extracted from the resulting clonal cell lines using QuickExtract DNA extraction solution (Biocentre), and the targeted region was PCR amplified, purified, and sequenced to detect indel mutations. At least three mutant clones and three isogenic, negative control clones (processed identically but found to be unmodified) were selected for further expansion and analysis. The primers used for amplification and sequencing analysis were the following: 5′-GAATCCAGGGGAGAAATTACATCACAG-3′ (SEQ ID NO: 4) and 5′-AAAGCGGAGGGTGATACAAGGTAAAAG-3′ (SEQ ID NO: 5) for KIF3A; 5′-GGCTAGCCAACAACACTGGT-3′ (SEQ ID NO: 6) and 5′-AACAAGTGGTCGGAACGTCT-3′ (SEQ ID NO: 7) for KIF3B gRNA 1; or 5′-AAAGGGACGGCCCATGAAAT-3′ (SEQ ID NO: 8) and 5′-CGTTCATGTTGGTAGCACCG-3′ (SEQ ID NO: 9) for KIF3B gRNA 2. Amplified fragments were used to clone individual alleles into the pCR™ 4-TOPO@ plasmid using a TOPO TA cloning kit for sequencing (Thermo Fisher). Plasmids were sequenced using M13 Forward primer (5′-GTAAAACGACGGCCAG-3′) (SEQ ID NO: 10) and chromatograms were aligned to a sequence from control, non-edited cells to identify indel mutations.

### Pluripotent Stem Cell Culture

The pluripotent stem cell lines were maintained in feeder-free culture conditions in 5% carbon dioxide with daily medium changes of mTesR1 (STEMCELL Technologies). For cilia detection, dissociated cells were plated into 8-well chamber slides (Lab-Tek) coated with 3% Geltrex and grown to confluency for 6 days with daily mTesR1 medium changes. To promote ciliogenesis, cells were treated with 100 μM cAMP for 48 hours and the medium was changed to DMEM/F12 for the last 24 hours of culture. For cavitated spheroid formation, hPSC were dissociated into single cells and plated at low density in 96-well plates coated with 1% Geltrex. The next morning, mTeSR1 containing 1.5% Geltrex was added to the wells. The cells were fixed with 4% paraformaldehyde 48 hours later. For monitoring cell viability, cells were plated in 96-wells and cultured 24 hours in 90 μL mTesR1 supplemented with 10 μL of alamarBlue cell viability reagent (Thermo Fisher). Fluorescence was read using a fluorescence excitation wavelength of 560 nm and an emission of 590 nm in a Perkin Elmer Envision Xcite instrument. Media was replaced with fresh mTesR1 supplemented with 10 μL of alamarBlue daily after the fluorescence measurements for the duration of the experiment.

### Protein Gels and Immunoblotting

KIF3A^{-/-}, KIF3B^{-/-} and isogenic control hPSCs were lysed with RIPA buffer containing protease and phosphatase inhibitors (Roche). Protein concentration was determined using a Pierce BCA protein assay kit. 50 μg of total protein was separated in a 4-20% acrylamide gel (Bio-Rad) and analyzed with silver stain (Bio-Rad) or transferred onto a PDVF membrane using standard procedures. 5% milk was used as a blocking agent prior to and during immunoblotting. Blots were probed with antibodies raised against PC1 (Santa Cruz sc-130554), PC2 (Santa Cruz sc-25749), KIF3A (Abcam ab11259), KIF3B (Cell Signaling 13817), α-Actin (Cell Signaling 4970), FLOT1 (Cell Signaling 18634), IFT88 (Proteintech 13967-1-AP), ARL13B (Proteintech 17711-1-AP), GLI1 (Cell Signaling 3538), GLI2 (Abcam ab26056), PTCH1 (Cell Signaling 8358), ALIX (Cell Signaling 2171), ANXA5 (Cell Signaling 8555), GM130 (Cell Signaling 12480), HPS70 (Cell Signaling 4876), CD9 (Cell Signaling 13174), ICAM-1 (Cell Signaling 4915), and EpCAM (Cell Signaling 2626).

### Kidney Organoid Differentiation

Kidney organoids were generated as previously described (Freedman, B. S. et al. Modelling kidney disease with CRISPR-mutant kidney organoids derived from human pluripotent epiblast spheroids. Nat Commun 6, 8715 (2015)). Briefly, dissociated hPSCs were plated into 96 or 24 well plates coated with 1% Geltrex (Thermo Fisher) in mTeSR1 (STEMCELL Technologies) supplemented with 10 μM Rho-kinase inhibitor Y27632 (Tocris Bioscience). The next morning, mTeSR1 containing 1.5% Geltrex was added to the wells, and the media was changed again to mTeSR1 24 hours later. 72 hours after plating, cells were treated with 12 μM CHIR99021 (Stemgent) in RPMI medium containing 1× Glutamax (Thermo Fisher) with 2 ng/ml recombinant BMP-4 (Peprotech) to induce kidney differentiation. 36 hours after treatment, the medium was changed to RPMI supplemented with B27 (RB medium, Thermo Fisher). RB medium changes were performed every three days. When indicated, organoid cultures were supplemented with 1 μM SAG or 10 μM Cyclopamine (both from Cayman Chemical) starting with the first addition of RB on day 1.5 (36 hours after CHIR99021 treatment on day 0) and maintained thereafter in RB media containing these compounds at all subsequent time points.

For suspension cultures, organoids in adherent cultures on day 21 of differentiation were selected for microdissection based on their three-dimensional, translucent, tubular appearance as detected by phase contrast microscopy. The organoids were gently detached from the plate using a 22 gauge needle and transferred into low adhesion plates (Corning) using a plastic transfer pipet. Organoids microdissected from cultures of mutants and isogenic controls were confirmed to be of similar size and morphology at the time of transfer. Media was replaced weekly, and cultures were maintained for one year. The 1 year old organoids are visible to the naked eyes and were photographed using a handheld digital camera. The images were opened with FIJI for quantification. The freehand selection tool was used to trace the organoid and measure the area of the organoid. For immunoblot analysis, microdissected organoids were transferred into an Eppendorf tube, allowed to settle by gravity, washed once with a full tube volume of PBS, allowed to settle, and subsequently lysed in the tube.

### Generation of Embryoid Bodies (EBs)

To generate EBs, 400,000 dissociated hPSCs were transferred into one well of a low-adhesion 6-well plate (Corning) in mTeSR1 plus 10 μM Rho-kinase inhibitor Y27632 (Tocris Bioscience). The following day, the cells were allowed to settle and the media was changed to mTeSR1 without Y27632. Two days later, the media was exchanged to ESC media (20% Knock Out Serum Replacement, 1× nonessential amino acids, 1× Glutamax, 1× penicillin-streptomycin, and 0.1 mM β-mercaptoethanol, in DMEM/F12, all from Thermo Fisher) without FGF. Media was changed every four days for the following eight weeks, after which the EBs were transferred onto gelatin-coated 24-well plates in 10% fetal bovine serum in DMEM and grown as outgrowths for two additional weeks. Floating EBs or EB outgrowths were subsequently fixed and processed for immunofluorescence analysis.

### Teratoma Formation

Animal work was performed in compliance with the strict ethical requirements and regulations of the UW IACUC under a pre-approved animal protocol. Dissociated hPSCs (400,000/well) were plated in three wells of a 6-well plate and grown to confluence in mTeSR1. Cells were dissociated, pelleted, resuspended in 500 μL of an ice-cold 1:1 mixture of DMEM/F12 (Fisher) and Matrigel (Corning). The cells were immediately injected beneath the neck scruff of immunodeficient, NOD-SCID mice (NOD.CB17-Prkdc^{scid}/J, Jackson Labs) using a 22-gauge syringe needle. Littermate animals of equally mixed genders at 8 weeks of age were used for all experiments. Growths were harvested 15 weeks after injection, photographed, fixed in methacarn (60% methanol, 30% chloroform, 10% acetic acid, all from Sigma), embedded in paraffin, sectioned, and stained with hematoxylin and eosin for histological analysis.

### Immunofluorescence Analysis

Cells and organoid cultures were fixed with 4% paraformaldehyde (Electron Microscopy Sciences) for 15 minutes, washed 3 times with Phosphate buffered saline (PBS, Thermo Fisher), blocked for 1 hour with blocking buffer (5% normal donkey serum and 0.3% Triton X-100 in PBS), and incubated overnight with primary antibodies including mouse acetylated α-tubulin (Sigma T7451), rabbit acetylated α-tubulin (Cell Signaling, D20G3), α-fetoprotein (AFP) (R&D MAB1368), α-smooth muscle actin (SMA) (R&D MAB1420), ZO-1 (Invitrogen, 339100), E-Cadherin (Abcam, ab11512), Nephrin (R&D, AF4269), p-Histone H3 (Santa Cruz sc-8656-R), OCT4 (Abcam ab19857), brachyury (BRY) (Santa Cruz sc-17745), SOX2 (Abcam ab97959), and 3-Tubulin III (Tuj1) (Sigma-Aldrich T2200-200UL). After washing with PBS, secondary donkey antibodies conjugated with Alexa-Fluor 488, 555, or 647 (1:500, Thermo Fisher) were applied, or Fluorescein Lotus Lectin (Vector Labs, FL-1321), with DAPI to stain the nuclei. Stained cells were imaged in PBS using a Nikon AIR confocal microscope or Eclipse TE wide-field microscope.

### EV Purification

1.5×10⁶ control or kinesin-2 knockout hPSCs were plated onto 10 cm diameter plates and grown to confluence in mTeSR1 (10 ml/plate). Alternatively, organoids were cultured in RB media (4 ml/well) for seven days. The media was collected without cells and centrifuged at 2,000 g in a swinging bucket tabletop centrifuge in 50 ml conical tubes (Falcon) for 10 minutes to pellet any residual cells and cellular debris. The supernatant was then transferred to thick-walled polypropylene round-bottom tubes (Beckman #355642) and EV were pelleted at 17,000 g at 4 degrees Celsius for 30 minutes in an RC-6 Plus centrifuge using an HB-6 swinging bucket rotor (Sorvall). The supernatant was aspirated and the pellet was resuspended in 100 μL of RIPA buffer containing protease and phosphatase inhibitors (Roche).

### Quantification and Statistics

Differentiation, processing, and analysis of mutant and control hPSCs was performed side-by-side. Multiple clones of the KIF3A^{-/-} or KIF3B^{-/-} genotypes were utilized in each experiment to control for any variability between clonal cell lines. Table 1 details the use of multiple clones for each experiment.

TABLE 1
Lines and n used for statistical analysis.

| | | | Total | KIF3A | | KIF3B | | Control | |
|---|---|---|---|---|---|---|---|---|---|
| Figure | Panel | Total n≥ | lines | n | Lines | n | Lines | n | Lines |
| 1 | E | 7 | 24 | 7 | 7 | 7 | 7 | 10 | 10 |
| 1 | G | 3 | 9 | 3 | 3 | 3 | 3 | 3 | 3 |
| 1 | H | 10 | 11 | 10 | 3 | 14 | 2 | 11 | 6 |
| 1 | I | 4 | 8 | 10 | 2 | 10 | 2 | 20 | 4 |
| 2 | B | 3 | 8 | 3 | 3 | 3 | 2 | 4 | 3 |
| 2 | D | 2 | 9 | 5 | 3 | 2 | 2 | 7 | 4 |
| 2 | E | 6 | 14 | 7 | 4 | 6 | 4 | 13 | 6 |
| 2 | G | 5 | 14 | 7 | 4 | 5 | 4 | 12 | 6 |
| 2 | H | 5 | 14 | 7 | 4 | 5 | 4 | 12 | 6 |
| 2 | J | 9 | 8 | 9 | 2 | 11 | 2 | 18 | 4 |
| 3 | C | 3 | 11 | 3 | 3 | 9 | 3 | 13 | 5 |
| 3 | F | 7 | 13 | 10 | 4 | 7 | 4 | 12 | 5 |
| 5 | D | 3 | 8 | 4 | 3 | 3 | 2 | 4 | 3 |
| 5 | F | 4 | 4 | | | | | 4 | 4 |
| 5 | G | 10 | 4 | | | | | 10 | 4 |
| 5 | G | 10 | 4 | | | | | 10 | 4 |
| 6 | C | 4 | 10 | 5 | 3 | 4 | 2 | 10 | 5 |
| 7 | F | 6 | 14 | 6 | 4 | 6 | 4 | 8 | 6 |
| 7 | G | 3 | 6 | 3 | 3 | 2 | 2 | 4 | 4 |
| 7 | I | 3 | 6 | | | 3 | 3 | 3 | 3 |

Fluorescence intensity analysis of BRY and OCT4 was performed on images processed and imaged identically, individual cells were identified automatically using Cell Profiler 2.0 and the average intensity per cell was plotted in BBP. Line scan analyses were conducted utilizing the Intensity Profile function in NIS Elements. A single line scan of equal length was performed on each structure, and subsequently these were averaged to generate the graphs, plotting pixel intensity values for each channel on the y-axis against the points along the line on the x-axis. Immunoblot band intensity levels were quantified using the ImageJ magic wand tool, normalized to the loading control, and pooled from multiple experiments to determine average and S.E.M. values. A two-tailed t-test for two samples with unequal variance was applied to determine significance. In cases where a treatment condition was applied, a paired t-test was performed. Statistical analyses were performed in GraphPad Prism 9 software.

### Differential Gene Expression and Gene-Set Enrichment Analysis

To minimize batch effects, all cells were prepared in a single batch. For each condition, ˜1000 cells were lysed directly in SMARTseq lysis buffer. Library was prepared from small using a combination of Clontech SMARTseq chemistry and Illumina Nextera XT kits. RNA was sequenced on an Illumina HiSeq2500 processor in one direction (single read), with a target of 5M reads per sample for 58 nucleotides (Genomics Core, Benaroya Research Institute, Seattle, WA). Differential expression analysis was performed using the DESeq2 R package (Love, M. I., Huber, W. & Anders, S. Moderated estimation of fold change and dispersion for RNA-seq data with DESeq2. Genome Biol 15, 550 (2014)). For all analyses, p-values were corrected for multiple hypothesis testing using the Benjamini-Hochberg procedure. For the joint analysis of KIF3A^{-/-}, KIF3B^{-/-} and control RNA-seq samples, differential expression due to loss of KIF3 activity was assessed using a model of “expression ˜KIF3 status”. To leverage our higher statistical power to identify signatures associated with KIF3B loss in KIF3B^{-/-} hPSCs, we performed differential gene expression analysis on KIF3B^{-/-} and control RNA-seq samples using a model of “expression ˜ KIF3B^{-/-} status”. For both analyses, hierarchical clustering was performed to group differentially expressed genes with similar dynamics across samples using the pheatmap R package specifying ward. D2 as the clustering method. Gene set enrichment analysis was performed on clusters of differentially expressed genes as a function of KIF3B^{-/-} loss using the hypergeometric test implemented in the piano R package.

### Example 2

This Example describes differentiation of genetically modified kidney organoids from genetically modified hPSCs can be performed in a reproducible manner following the adherent culture protocol developed by the inventor. See e.g., the organoid differentiation protocol described in the following references all of which are incorporated herein by reference (Cruz, N. M., and Freedman, B. S., Chapter 7—“Differentiation of human kidney organoids from pluripotent stem cells, Methods in Cell Biology”, Editor: Weimbs, T, Academic Press, vol. 153, 2019, pages 133-150; Cruz, N. M., Song, X., Czemiecki, S. M., Gulieva, R. E., Churchill, A. J., Kim, Y. K., et al. (2017). Organoid cystogenesis reveals a critical role of microenvironment in human polycystic kidney disease. Nature Materials, 16, 1112-1119; Czemiecki, S. M., Cruz, N. M., Harder, J. L., Menon, R., Annis, J., Otto, E. A., et al. (2018). High-throughput screening enhances kidney organoid differentiation from human pluripotent stem cells and enables automated multidimensional phenotyping. Cell Stem Cell, 22(6), 929-940. e924; Freedman, B. S., Brooks, C. R., Lam, A. Q., Fu, H., Morizane, R., Agrawal, V., et al. (2015). Modelling kidney disease with CRISPR-mutant kidney organoids derived from human pluripotent epiblast spheroids. Nature Communications, 6, 8715; and Kim, Y. K., Refaeli, I., Brooks, C. R., Jing, P., Gulieva, R. E., Hughes, M. R., et al. (2017). Gene-edited human kidney organoids reveal mechanisms of disease in podocyte development. Stem Cells, 35(12), 2366-2378).

With respect to the outcome, the protocols described in any of the references incorporated above result in ≥50% of the product being kidney organoids. The hallmark of the cultures is the presence of nephron-like structures unique to the kidneys (about 50% of the cells). But there are also stromal cells, neurons, blood vessel endothelial cells. These are still considered to be part of the ‘organoid’ (or perhaps better described ‘organoid culture’).

In the case of the cilia knockout lines (see e.g., Example 1), they are differentiated into these organoid cultures the exact same way as described in the protocols from any of the references incorporated above. They still grow and form organoid structures. This happens reproducibly, and to one of ordinary skill in the art, this requires no extra description in words beyond the established method described below.

The composition of the organoid cultures formed from cilia knockout cells are somewhat different from organoid cultures formed from wild-type cell lines. For example, structures and cell types may be present in different proportions. They can also represent less mature states that have not been fully characterized or figured out exactly how they are different from organoids with cilia. These organoid cultures are mutants and therefore they are different in form. Nevertheless, the genetically modified organoid cultures routinely and reproducibly form nephron-like structures along with the other cell types in clearly recognizable forms.

While illustrative embodiments have been illustrated and described, it will be appreciated that various changes can be made therein without departing from the spirit and scope of the invention.

1. An isolated genetically modified pluripotent stem cell (PSC) comprising a disruption in a gene encoding at least one subunit of kinesin-2, wherein the genetic modification results in the PSC lacking a ciliary structure.

2. The isolated genetically modified PSC of claim 1,
wherein the genetic modification comprises disruption of KIF3A and reduced expression of a KIF3A protein, wherein reduced expression of a KIF3A protein results in the PSC lacking a ciliary structure, or
wherein the genetic modification comprises disruption of KIF3B and reduced expression of a KIF3B protein, wherein reduced expression of a KIF3B protein results in the PSC lacking a ciliary structure, or
wherein the genetic modification comprises disruption of KIF3A and KIF3B, wherein reduced expression of a KIF3A protein and a KIF3B protein result in the PSC lacking a ciliary structure.

3-4. (canceled)

5. The genetically modified PSC of claim 1, wherein the PSC is a human PSC (hPSC), and/or
wherein the PSC is an embryonic stem cell (ESC), and/or
wherein the PSC is an induced PSC (iPSC).

6-8. (canceled)

9. The genetically modified PSC of claim 1, wherein the PSC lacks a ciliary axoneme, and/or
wherein the PSC lacks primary cilia, multi-ciliated arrays, or primary cilia and multi-ciliated arrays.

10. (canceled)

11. A genetically modified cell descended from the genetically modified PSC of claim 1.

12. (canceled)

13. An isolated genetically modified organoid derived from the genetically modified PSC of claim 1.

14. The isolated genetically modified organoid of claim 13,
wherein the organoid comprises neuronal structures as determined based on axon-like rosette morphology and gene expression markers such as SOX2 and TUJ1, and/or
wherein the organoid comprises nephron structures comprising distal tubules, proximal tubules, or podocytes in contiguous arrangements with appropriate morphology and markers, optionally wherein the organoid comprises epithelial cells positive for ECAD+, LTL+, PODXL+, or a combination of ECAD+, LTL+, PODXL+.

15-27. (canceled)

28. A method of differentiating an isolated genetically modified tubular organoid derived from a genetically modified pluripotent stem cell (PSC), the method comprising:
(a) providing a quantity of genetically modified PSCs, wherein the genetically modified PSCs comprise a target gene encoding at least one subunit of kinesin-2 and contain a disruption of the target gene that results in the PSCs lacking a ciliary structure;
(b) culturing the genetically modified PSCs in a first culture medium comprising a ROC kinase inhibitor for at least 24 hours and then culturing the PSC sandwiched between two layers of a gelatinous protein mixture secreted by Engelbreth-Holm-Swarm mouse sarcoma cells to form epiblast spheroids, wherein the first medium does not comprise exogenous fibroblast growth factor 2 (FGF2), activin or bone morphogenetic protein; and
(c) contacting the epiblast spheroids from step (b) with a second culture medium comprising at least 12 μM CHIR99021 for at least 24 hours, wherein the second medium does not comprise exogenous fibroblast growth factor 2 (FGF2), activin or bone morphogenetic protein, and
(d) culturing the epiblast spheroids from step (c) with a third culture medium comprising B27 for at least 48 hours, wherein the third medium does not comprise exogenous fibroblast growth factor 2 (FGF2), activin or bone morphogenetic, thereby differentiating the epiblast spheroids into genetically modified tubular organoids, wherein the genetically modified tubular organoids lack a ciliary structure.

29. The method of claim 28,
wherein the genetically modified PSCs of step (b) are cultured in a medium lacking leukemia inhibitory factor (LIF) and doxycycline prior to forming epiblast spheroids, and/or
wherein the genetically modified PSCs in step (b) are cultured in the first culture medium comprising the ROC kinase inhibitor for at least 48 hours.

30-31. (canceled)

32. The method of claim 28, wherein the genetically modified tubular organoids are genetically modified kidney organoids.

33. (canceled)

34. The method of claim 28, wherein
the genetically modified tubular organoids express at least one of podocalyxtin (PODXL), zonula occluden (ZO-1), and lotus tetragonolobus lectin (LTL).

35. A method for testing the effects of compound candidates on a phenotypic organoid model, the method comprising:
generating the phenotypic organoid model on a screening platform comprising the steps of:
(a) plating each of a plurality of wells of a high throughput culture vessel with a population of genetically modified pluripotent stem cells (PSCs) according to claim 1;
(b) differentiating the population of genetically modified PSCs plated in each of the plurality of wells using a single induction step without dissociating or replating the differentiated cells;
treating the population of genetically modified PSCs plated in each of the plurality of wells with a therapeutic compound candidate; and
testing one or more effects resulting from treatment with each of the therapeutic compound candidates;
wherein the method is performed manually using pipettes or automatically by a liquid handling robot.

36-38. (canceled)

39. The method of claim 35, wherein the single induction step comprises treating the population of genetically modified PSCs in each of the plurality of wells with a concentration of CHIR99021.

40-41. (canceled)

42. The method of claim 35, wherein the one or more effects resulting from treatment with each of the therapeutic compound candidates include cell toxicity, cell differentiation, or efficacy.

43. The method of claim 35, wherein generating the phenotypic organoid model further comprises treating the population of cells with VEGF.

44. A method for measuring organ specific toxicity and disease phenotypes of an agent on a genetically modified organoid lacking a ciliary structure, the method comprising:
(a) providing one or more genetically modified organoids lacking a ciliary structure and derived from a genetically modified pluripotent stem cell (PSC) in a high throughput format;
(b) admixing the agent with the one or more genetically modified organoids; and
(c) detecting one or more outcomes of agent on the one or more genetically modified organoids wherein the one or more outcomes indicates toxicity, disease, differentiation state, or a combination thereof of the one or more genetically modified organoids.

45-47. (canceled)

48. The method of claim 44, wherein the one or more genetically modified organoids are derived from human iPSCs.

49. The method of claim 44, wherein the one or more organoids are genetically modified kidney organoids.

50. The method of claim 44, further comprising performing single-cell RNA-seq on the one or more genetically modified organoids.

51. The method of claim 44, wherein the one or more outcomes comprises phenotypic screening of the one or more genetically modified organoids.

52. (canceled)


SEQUENCE LISTING (36 sequences)

SEQ ID NO: 1 - DNA, 19 bp, unidentified
  source 1..19: mol_type = other DNA
catatggacaaaccggaac

SEQ ID NO: 2 - DNA, 19 bp, unidentified
  source 1..19: mol_type = other DNA
ttcgctgtcggcccatgaa

SEQ ID NO: 3 - DNA, 20 bp, unidentified
  source 1..20: mol_type = other DNA
tacaccatggaaggaatccg

SEQ ID NO: 4 - DNA, 27 bp, unidentified
  source 1..27: mol_type = other DNA
gaatccaggggagaaattacatcacag

SEQ ID NO: 5 - DNA, 27 bp, unidentified
  source 1..27: mol_type = other DNA
aaagcggagggtgatacaaggtaaaag

SEQ ID NO: 6 - DNA, 20 bp, unidentified
  source 1..20: mol_type = other DNA
ggctagccaacaacactggt

SEQ ID NO: 7 - DNA, 20 bp, unidentified
  source 1..20: mol_type = other DNA
aacaagtggtcggaacgtct

SEQ ID NO: 8 - DNA, 20 bp, unidentified
  source 1..20: mol_type = other DNA
aaagggacggcccatgaaat

SEQ ID NO: 9 - DNA, 20 bp, unidentified
  source 1..20: mol_type = other DNA
cgttcatgttggtagcaccg

SEQ ID NO: 10 - DNA, 16 bp, unidentified
  source 1..16: mol_type = other DNA
gtaaaacgacggccag

SEQ ID NO: 11 - DNA, 33 bp, unidentified
  source 1..33: mol_type = other DNA
ttgcatatggacaaaccggaacaggcaaaactt

SEQ ID NO: 12 - DNA, 31 bp, unidentified
  source 1..31: mol_type = other DNA
tggtggttcgctgtcggcccatgaatggcaa

SEQ ID NO: 13 - DNA, 28 bp, unidentified
  source 1..28: mol_type = other DNA
ttgcatatggacaaaacaggcaaaactt

SEQ ID NO: 14 - DNA, 20 bp, unidentified
  source 1..20: mol_type = other DNA
tggtggttcgctgtcggcaa

SEQ ID NO: 15 - DNA, 28 bp, unidentified
  source 1..28: mol_type = other DNA
tttttgcatatggacaaaccggaacagg

SEQ ID NO: 16 - DNA, 28 bp, unidentified
  source 1..28: mol_type = other DNA
tttttgcatatggacaaaccggcaaaca

SEQ ID NO: 17 - DNA, 23 bp, unidentified
  source 1..23: mol_type = other DNA
tttttgcatatggacaaaacagg

SEQ ID NO: 18 - DNA, 28 bp, unidentified
  source 1..28: mol_type = other DNA
tttttgcatatggacaaaccgggaacag

SEQ ID NO: 19 - DNA, 27 bp, unidentified
  source 1..27: mol_type = other DNA
ggttcgctgtcggcccatgaatggcaa

SEQ ID NO: 20 - DNA, 27 bp, unidentified
  source 1..27: mol_type = other DNA
ggttcgctgtcggcccattgaatggca

SEQ ID NO: 21 - DNA, 16 bp, unidentified
  source 1..16: mol_type = other DNA
ggttcgctgtcggcaa

SEQ ID NO: 22 - DNA, 27 bp, unidentified
  source 1..27: mol_type = other DNA
ggttcgctgtcggcccattgaatggca

SEQ ID NO: 23 - DNA, 27 bp, unidentified
  source 1..27: mol_type = other DNA
aacctacaccatggaaggaatccgtgg

SEQ ID NO: 24 - DNA, 14 bp, unidentified
  source 1..14: mol_type = other DNA
aacctacaccatgg

SEQ ID NO: 25 - DNA, 30 bp, unidentified
  source 1..30: mol_type = other DNA
aacctacaccatggaaggaaggaattccgt

SEQ ID NO: 26 - DNA, 31 bp, unidentified
  source 1..31: mol_type = other DNA
cagggactttttccatgtaaggagttcgagc

SEQ ID NO: 27 - DNA, 34 bp, unidentified
  source 1..34: mol_type = other DNA
cagggactatttttgcatatggacaaaccgggaa

SEQ ID NO: 28 - DNA, 17 bp, unidentified
  source 1..17: mol_type = other DNA
gcatatggacaaaccgg

SEQ ID NO: 29 - DNA, 25 bp, unidentified
  source 1..25: mol_type = other DNA
gcatatggacaaaccggaaaacttt

SEQ ID NO: 30 - DNA, 18 bp, unidentified
  source 1..18: mol_type = other DNA
gcatatggacaaaacttt

SEQ ID NO: 31 - DNA, 10 bp, unidentified
  source 1..10: mol_type = other DNA
ttgcatattt

SEQ ID NO: 32 - DNA, 31 bp, unidentified
  source 1..31: mol_type = other DNA
ttgcatatggacaaaccaacaggcaaaactt

SEQ ID NO: 33 - DNA, 11 bp, unidentified
  source 1..11: mol_type = other DNA
ttgcaaaactt

SEQ ID NO: 34 - DNA, 31 bp, unidentified
  source 1..31: mol_type = other DNA
ggttcgctgtcggcccattgaatgggaagga

SEQ ID NO: 35 - DNA, 31 bp, unidentified
  source 1..31: mol_type = other DNA
ggttcgctgtcggcccattgaatggcaagga

SEQ ID NO: 36 - DNA, 18 bp, unidentified
  source 1..18: mol_type = other DNA
ggttcgcaatggcaagga